(19) United States
(12) Patent Application Publication
(10) Pub. No.: US 2006/0204503 A1
(43) Pub. Date: Sep. 14, 2006

(54) Treatment of cancer using antibodies to polypeptides differentially expressed in human lung tumors

(75) Inventors: Jonathon Fitchett, San Marcos, CA (US); Mark Daniels, San Diego, CA (US); Michael J. Labarre, San Diego, CA (US); Karen McLachlan, Encinitas, CA (US); Tony Rowe, Sandringham (AU); Nicole W. O'Brien, San Marcos, CA (US); Samuel Y. Cho, San Diego, CA (US); Melissa Heller, San Diego, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 11/343,271

(22) Filed: Jan. 31, 2006

Related U.S. Application Data
(60) Provisional application No. 60/648,257, filed on Jan. 31, 2005.

(51) Int. Cl. A61K 39/395; C07K 16/30; C40B 40/10; C12P 21/04

(52) U.S. Cl. 424/155.1; 530/388.8; 435/7.1; 435/70.21

(57) ABSTRACT

The present invention is directed to novel methods of treating, indentifying or diagnosing a hyperproliferative disorder in a patient in need thereof. The methods of the invention include administering to a patient a composition comprising a binding molecule which binds to a cell surface expressed glycoprotein expressed predominantly in tumor or tumor-associated cells. In particular, the therapeutic and diagnostic methods of the present invention include the use of a binding molecule, for example an antibody or immunospecific fragment thereof, which specifically binds to a lung tumor-associated polypeptide, variant polypeptide or fragment thereof.

### CROSS-REFERENCE TO RELATED APPLICATIONS

The present application claims the benefit of the filing date of U.S. Provisional Application No. 60/648,257, filed Jan. 31, 2005, which is incorporated herein by reference in its entirety.

### REFERENCE TO A SEQUENCE LISTING SUBMITTED ON A COMPACT DISC

This application includes a “Sequence Listing,” which is provided as an electronic document on a compact disc (CD-R). This compact disc contains the file “Sequence Listing ASCII, Docket No. 2159.0300001.ST25.txt” (253 kilobytes, created on Jan. 30, 2006), which is hereby incorporated by reference in its entirety.

### BACKGROUND OF THE INVENTION

1. Field of the Invention

The present invention is directed to novel methods of treating and diagnosing hyperproliferative disorders utilizing binding molecules which bind to polypeptides expressed predominantly in tumor or tumor-associated cells.

2. Background Art

Cancer afflicts approximately 1.2 million people in the United States each year. About 50% of these cancers are curable with surgery, radiation therapy, and chemotherapy. Despite significant technical advances in these three types of treatments, each year more than 500,000 people will die of cancer in the United States alone. (Jaffee, E. M., Ann. N.Y. Acad. Sci. 886:67-72 (1999)). Because most recurrences are at distant sites such as the liver, brain, bone, and lung, there is an urgent need for improved systemic therapies.

Advances have been made in detection and therapy of cancer, however no vaccine or other universally successful method for prevention or treatment is currently available. One reason for failure of a cancer treatment is often the growth of secondary metastatic lesions in distant organs. Therapy for metastasis currently relies on a combination of early diagnosis and aggressive treatment, which may include radiotherapy, chemotherapy or hormone therapy. However, the toxicity of such treatments limits the use of presently available anticancer agents for treatment of malignant disease. The high mortality rate for many cancers indicates that improvements are needed in metastasis prevention and treatment. The goal of cancer treatment is to develop modalities that specifically target tumor cells, thereby avoiding unnecessary side effects to normal tissue. Immunotherapy has the potential to provide an alternative systemic treatment for most types of cancer. The advantage of immunotherapy over radiation and chemotherapy is that it can act specifically against the tumor without causing normal tissue damage.

The development of less toxic antitumor agents would facilitate the long term treatment of latent or residual disease. Such agents could also be used prophylactically after the removal of a precancerous tumor.

Accordingly, there is a need in the art for the development of further methods for detecting, inhibiting, and treating cancer, e.g., metastasis.

### BRIEF SUMMARY OF THE INVENTION

This invention involves the purification of membrane proteins that are upregulated in tissues associated with lung tumors. The invention also involves using protein separation and sequencing methods to determine amino acid sequences of peptides derived from membrane proteins of human tumor tissue samples, and comparing the peptide sequences to databases containing known protein amino acid sequences to identify the purified proteins, and to further identify proteins that are specifically present in lung tumor tissues as well as other tissues associated with a hyperproliferative disease or disorder. When proteins of non-human tissues are identified, the invention also involves comparing the amino acid sequences of such proteins to the databases to identify their human homologs.

In one embodiment, the present invention provides a method for treating a hyperproliferative disorder in an animal, comprising administering to an animal in need of treatment a composition comprising a binding molecule which specifically binds to lung tumor-associated polypeptide, variant or fragment thereof.

In another embodiment the invention provides a method of detecting abnormal hyperproliferative cell growth in a patient, comprising: obtaining a biological sample from the patient; contacting the sample with a binding molecule which specifically binds to lung tumor-associated polypeptide, variant or fragment thereof, and assaying the expression level of the lung tumor-associated polypeptide in the sample.

Yet another embodiment provides a method of diagnosing a hyperproliferative disease or disorder in a patient, comprising administering to the patient a sufficient amount of a detectably labeled binding molecule which specifically binds to a lung tumor-associated polypeptide, variant or fragment thereof, waiting for a time interval following the administration to allow the binding molecule to contact the lung tumor-associated polypeptide, variant or fragment thereof and detecting the amount of binding molecule which is bound to the lung tumor-associated polypeptide, variant or fragment thereof in the patient.

In various embodiments, binding molecules for use in the above methods include antibodies and antigen-specific fragments thereof, fusion proteins, T-cell receptors, and small molecules.

In the above methods, binding molecules bind to polypeptide variants or fragments there of which are at least 70% identical to lung tumor-associated polypeptides selected from the group consisting of SEQ ID NOs: 1 to 52. Additionally, the binding molecules of the above methods bind to polypeptide variants or fragments which comprise specific domains of the lung tumor-associated polypeptides or the extracellular domains of the lung tumor-associated polypeptides.

The invention further involves preparing therapeutic agents such as monoclonal antibodies and fusion proteins bearing extracellular binding domains that bind with high affinity and specificity to proteins that are specifically present in disease- or disorder-associated tissues, e.g., proteins that are useful targets for killing or interfering with the function of cells of the tissue that express the targeted proteins.

### BRIEF DESCRIPTION OF THE DRAWINGS/FIGURES

FIG. 1 —A) Total Ion Chromotography of gel slice 8 from tumor 1. B) Mass Spectrometry (MS) spectra for SEQ ID NO:1. C) Mass Spectrometry-Microsequencing (MS/MS) spectra for SEQ ID NO:1.

FIG. 2:—A) Total Ion Chromotography of gel slice 11 from tumor 3. B) MS spectra for SEQ ID NO:2. C) MS/MS spectra for SEQ ID NO:2.

FIG. 3:—A) Total Ion Chromotography of gel slice 17 from tumor 1. B) MS spectra for SEQ ID NO:3. C) MS/MS spectra for SEQ ID NO:3.

FIG. 4:—A) Total Ion Chromotography of gel slice 12 from tumor 3. B) MS spectra for SEQ ID NO:4. C) MS/MS spectra for SEQ ID NO:4.

FIG. 5:—A) Total Ion Chromotography of gel slice 9 from tumor 1. B) MS spectra for SEQ ID NO:5. C) MS/MS spectra for SEQ ID NO:5.

FIG. 6:—A) Total Ion Chromotography of gel slice 4 from tumor 1. B) MS spectra for SEQ ID NO:6. C) MS/MS spectra for SEQ ID NO:6.

FIG. 7:—A) Total Ion Chromotography of gel slice 7 from tumor 1. B) MS spectra for SEQ ID NO:7. C) MS/MS spectra for SEQ ID NO:7.

FIG. 8:—A) Total Ion Chromotography of gel slice 4 from tumor 1. B) MS spectra for SEQ ID NO:8. C) MS/MS spectra for SEQ ID NO:8.

FIG. 9:—A) Total Ion Chromotography of gel slice 14 from tumor 1. B) MS spectra for SEQ ID NO:9. C) MS/MS spectra for SEQ ID NO:9.

FIG. 10:—A) Total Ion Chromotography of gel slice 11 from tumor 1. B) MS spectra for SEQ ID NO:10. C) MS/MS spectra for SEQ ID NO:10.

FIG. 11:—A) Total Ion Chromotography of gel slice 15 from tumor 2. B) MS spectra for SEQ ID NO:11. C) MS/MS spectra for SEQ ID NO:11.

FIG. 12:—A) Total Ion Chromotography of gel slice 12 from tumor 2. B) MS spectra for SEQ ID NO:12 C) MS/MS spectra for SEQ ID NO:12.

FIG. 13:—Protein Structure Analysis of SEQ ID NO:25. A. Residue Schematic: each residue is represented by a line at the position it occurs in the sequence. B. Chou and Fasman Beta-Sheet Forming and Breaking Residues: a display of the residues that are beta-sheet forming and breaking as defined by Chou and Fasman (Adv. Enz. 47; 45-147 (1978)). C. Chou and Fasman Alpha and Beta Propensities: a plot of the Chou and Fasman propensity measures for alpha-helix and beta-sheet. D. Chou and Fasman Alpha-Helix Forming and Breaking Residues: residues that are alpha-helix forming and breaking, as defined by Chou and Fasman. E. Chou and Fasman Amino Ends: regions of the sequence that resemble sequences typically found at the amino end of alpha-helices and beta-structures. F. Chou and Fasman Carboxyl Ends: regions of the sequence typically found at the carboxyl end of alpha-helices and beta-structures. G. Chou and Fasman Turns: regions of the sequence typically found in turns. H. Hydrophobic Moment: the helical hydrophobic moment at each position of the sequence. I. Kyte and Doolittle Hydropathy: This curve is the average of a residue-specific hydrophobicity index over a window of nine residues. When the line is in the upper half of the frame, it indicates a hydrophobic region, and when it is in the lower half, a hydrophilic region. Panel I also includes Goldman, Engelman, and Steitz Transbilayer Helices curve for identifying nonpolar transbilayer helices (reviewed in Ann. Rev. Biophys. Biophys. Chem. 15; 321-353 (1986)). The curve is the average of a residue-specific hydrophobicity scale (the GES scale) over a window of 20 residues. When the line is in the upper half of the frame, it indicates a hydrophobic region and when it is in the lower half, ahydrophilic region.

FIG. 14:—Protein Structure Analysis of SEQ ID NO:26. Panels A-I are the same as described above for FIG. 13.

FIG. 15:—Protein Structure Analysis of SEQ ID NO:27. Panels A-I are the same as described above for FIG. 13.

FIG. 16:—Protein Structure Analysis of SEQ ID NO:28. Panels A-I are the same as described above for FIG. 13.

FIG. 17:—Protein Structure Analysis of SEQ ID NO:29. Panels A-I are the same as described above for FIG. 13.

FIG. 18:—Protein Structure Analysis of SEQ ID NO:30. Panels A-I are the same as described above for FIG. 13.

FIG. 19:—Protein Structure Analysis of SEQ ID NO:31. Panels A-I are the same as described above for FIG. 13.

FIG. 20:—Protein Structure Analysis of SEQ ID NO:32. Panels A-I are the same as described above for FIG. 13.

FIG. 21:—Protein Structure Analysis of SEQ ID NO:33. Panels A-I are the same as described above for FIG. 13.

FIG. 22:—Protein Structure Analysis of SEQ ID NO:34. Panels A-I are the same as described above for FIG. 13.

FIG. 23:—Protein Structure Analysis of SEQ ID NO:35. Panels A-I are the same as described above for FIG. 13.

FIG. 24:—Protein Structure Analysis of SEQ ID NO:36. Panels A-I are the same as described above for FIG. 13.

FIG. 25:—Protein Structure Analysis of SEQ ID NO:37. Panels A-I are the same as described above for FIG. 13.

FIG. 26:—Protein Structure Analysis of SEQ ID NO:38. Panels A-I are the same as described above for FIG. 13.

FIG. 27:—Protein Structure Analysis of SEQ ID NO:39. Panels A-I are the same as described above for FIG. 13.

FIG. 28:—Protein Structure Analysis of SEQ ID NO:40. Panels A-I are the same as described above for FIG. 13.

FIG. 29:—Protein Structure Analysis of SEQ ID NO:41. Panels A-I are the same as described above for FIG. 13.

FIG. 30:—Protein Structure Analysis of SEQ ID NO:42. Panels A-I are the same as described above for FIG. 13.

FIG. 31:—Protein Structure Analysis of SEQ ID NO:43. Panels A-I are the same as described above for FIG. 13.

FIG. 32:—Protein Structure Analysis of SEQ ID NO:44. Panels A-I are the same as described above for FIG. 13.

FIG. 33:—Protein Structure Analysis of SEQ ID NO:45. Panels A-I are the same as described above for FIG. 13.

FIG. 34:—Protein Structure Analysis of SEQ ID NO:46. Panels A-I are the same as described above for FIG. 13.

FIG. 35:—Protein Structure Analysis of SEQ ID NO:47. Panels A-I are the same as described above for FIG. 13.

FIG. 36:—Protein Structure Analysis of SEQ ID NO:48. Panels A-I are the same as described above for FIG. 13.

FIG. 37:—Protein Structure Analysis of SEQ ID NO:49. Panels A-I are the same as described above for FIG. 13.

FIG. 38:—Protein Structure Analysis of SEQ ID NO:50. Panels A-I are the same as described above for FIG. 13.

FIG. 39:—Protein Structure Analysis of SEQ ID NO:51. Panels A-I are the same as described above for FIG. 13.

FIG. 40:—Protein Structure Analysis of SEQ ID NO:52. Panels A-I are the same as described above for FIG. 13.

FIG. 41:—A) Total Ion Chromotography of gel slice 7 from tumor 1. B) MS spectra for SEQ ID NO:36. C) MS/MS spectra for SEQ ID NO:36. D) MS spectra for SEQ ID NO:30. E) MS/MS spectra for SEQ ID NO:30. F) MS spectra for SEQ ID NO:31. G) MS/MS spectra for SEQ ID NO:31. H) MS spectra for SEQ ID NO:38. I) MS/MS spectra for SEQ ID NO:38. J) MS spectra for SEQ ID NO:45. K) MS/MS spectra for SEQ ID NO:45.

FIG. 42: A) Total Ion Chromotography of gel slice 17 from tumor 1. B) MS spectra for SEQ ID NO:28. C) MS/MS spectra for SEQ ID NO:28. D) MS spectra for SEQ ID NO:47. E) MS/MS spectra for SEQ ID NO:47. F) MS spectra for SEQ ID NO:49. G) MS/MS spectra for SEQ ID NO:49.

FIG. 43: A) Total Ion Chromotography of gel slice 9 from tumor 1. B) MS spectra for SEQ ID NO:27. C) MS/MS spectra for SEQ ID NO:27. D) MS spectra for SEQ ID NO:46. E) MS/MS spectra for SEQ ID NO:46.

FIG. 44: A) Total Ion Chromotography of gel slice 14 from tumor 1. B) MS spectra SEQ ID NO:32. C) MS/MS spectra for SEQ ID NO:32.

FIG. 45: A) Total Ion Chromotography of gel slice 11 from tumor 1. B) MS spectra SEQ ID NO:44. C) MS/MS spectra for SEQ ID NO:44.

FIG. 46: A) TIC of gel slice 5 from tumor 1. B) MS spectra for SEQ ID NO:29. C) MS/MS spectra for SEQ ID NO:29.

FIG. 47: A) TIC of gel slice 7 from tumor 1. B) MS spectra for SEQ ID NO:42. C) MS/MS spectra for SEQ ID NO:42.

FIG. 48: A) TIC of gel slice 10 from tumor 2. B) MS spectra for SEQ ID NO:39. C) MS/MS spectra for SEQ ID NO:39. D) MS spectra for SEQ ID NO:41. E) MS/MS spectra for SEQ ID NO:41. F) MS spectra for SEQ ID NO:50. G) MS/MS spectra for SEQ ID NO:50.

FIG. 49: A) TIC of gel slice 11 from tumor 2. B) MS spectra for SEQ ID NO:48. C) MS/MS spectra for SEQ ID NO:48.

FIG. 50: A) TIC of gel slice 14 from tumor 2. B) MS spectra for SEQ ID NO:25. C) MS/MS spectra for SEQ ID NO:25. D) MS spectra for SEQ ID NO:35. E) MS/MS spectra for SEQ ID NO:35.

FIG. 51: A) TIC of gel slice 15 from tumor 2. B) MS spectra for SEQ ID NO:40. C) MS/MS spectra for SEQ ID NO:40.

FIG. 52: A) TIC of gel slice 16 from tumor 2. B) MS spectra for SEQ ID NO:34. C) MS/MS spectra for SEQ ID NO:34.

FIG. 53: A) TIC of gel slice 10 from tumor 3. B) MS spectra for SEQ ID NO:32. C) MS/MS spectra for SEQ ID NO:32. D) MS spectra for SEQ ID NO:26. E) MS/MS spectra for SEQ ID NO:26.

FIG. 54: A) TIC of gel slice 11 from tumor 3. B) MS spectra for SEQ ID NO:37. C) MS/MS spectra for SEQ ID NO:37.

FIG. 55: A) TIC of gel slice 10 from tumor 3. B) MS spectra for SEQ ID NO:43. C) MS/MS spectra for SEQ ID NO:43.

FIG. 56: A) TIC of gel slice 15 from tumor 3. B) MS spectra for SEQ ID NO:51. C) MS/MS spectra for SEQ ID NO:51.

FIG. 57: A) TIC of gel slice 7 from tumor 1. B) MS spectra for SEQ ID NO:52. C) MS/MS spectra for SEQ ID NO:52.

### DETAILED DESCRIPTION OF THE INVENTION

### DEFINITIONS

The following definitions are provided to facilitate understanding of certain terms used throughout the specification.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

In the present invention, “isolated” refers to material removed from its native environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “membrane protein” or “membrane polypeptide” is a polypeptide that is present in the membrane of cells through either direct or indirect association with the lipid bilayer, including, in particular, through prenylation of a carboxyl-terminal amino acid motif. Membrane proteins are amphipathic, meaning that the polypeptide has a hydrophobic and a hydrophilic region. Typically the hydrophobic regions interact with the lipid bilayer of the cell and the hydrophilic regions interact with the aqueous interior or exterior of the cell.

Certain membrane proteins are “transmembrane proteins” and have a extracellular domain, which interacts with the external cellular environment, an intracellular domain, which interacts with the internal cellular environment and a transmembrane domain which traverses the cellular lipid bilayer. Certain membrane proteins however do not have extracellular domains and interact with the lipid bilayer through covalently attached fatty acid groups, prenyl groups, oligosaccharides or through protein-protein interacts with other proteins in the cellular membrane. The addition of prenyl groups is known as prenylation and involves the covalent modification of a protein by the addition of either a farnesyl or geranylgeranyl isoprenoid. Prenylation occurs on a cysteine residue located near the carboxyl-terminus of a protein.

As used herein, a “polynucleotide” can contain the nucleotide sequence of the full length cDNA sequence, including the untranslated 5′ and 3′ sequences, the coding sequences, as well as fragments, eptiopes, domains, and variants of the nucleic acid sequence. The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In the present invention, a polypeptide can be composed of amino acids joined to each other by peptide bonds or modified peptide bonds, i.e., peptide isosteres, and may contain amino acids other than the 20 gene-encoded amino acids. The polypeptides of the present invention may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

In the present invention, a “polypeptide fragment” refers to a short amino acid sequence of the polypeptides of SEQ ID NOs: 1-52. Protein fragments may be “free-standing,” or comprised within a larger polypeptide of which the fragment forms a part of region. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids in length.

Binding Molecules. The methods of treating hyperproliferative disorders as described herein utilize “binding molecules.” A binding molecule comprises, consists essentially of, or consists of at least one binding domain which, either alone or in combination with one or more additional binding domains, specifically binds to a target gene product (such as a protein, an antigen or other binding partner), e.g., a lung tumor-associated polypeptide or fragment or variant thereof. For example, in various embodiments, a binding molecule comprises one or more immunoglobulin antigen binding domains, one or more binding domains of a receptor molecule which, either alone or together, specifically bind a ligand, or one or more binding domains of a ligand molecule which, either alone or together, specifically bind a receptor. In certain embodiments, a binding molecule comprises, consists essentially of, or consists of at least two binding domains, for example, two, three, four, five, six, or more binding domains. Each binding domain may bind to a target molecule separately, or two or more binding domains may be required to bind to a given target, for example, a combination of an immunoglobulin heavy chain and an immunoglobulin light chain.

Binding molecules, e.g., binding polypeptides, e.g., lung tumor-associated polypeptide-specific antibodies used in the diagnostic and treatment methods disclosed herein may comprise, consist essentially of, or consist of two or more subunits thus forming multimers, e.g., dimers, trimers or tetramers. For example, certain binding molecules comprise a polypeptide dimer, typically, a heterodimer comprising two non-identical monomeric subunits. Other binding molecules comprise tetramers, which can include two pairs of homodimers, e.g., two identical monomeric subunits, e.g., an antibody molecule consisting of two identical heavy chains and two identical light chains.

Certain binding molecules, e.g., binding polypeptides to be utilized in the diagnostic and treatment methods disclosed herein comprise at least one amino acid sequence derived from an immunoglobulin. A polypeptide or amino acid sequence “derived from” a designated protein refers to the origin of the polypeptide. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the starting sequence. Alternatively, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence, as described in more detail below.

Preferred binding polypeptides comprise, consist essentially of, or consist of an amino acid sequence derived from a human amino acid sequence. However, binding polypeptides may comprise one or more contiguous amino acids derived from another mammalian species. For example, a primate heavy chain portion, hinge portion, or binding site may be included in the subject binding polypeptides. Alternatively, one or more murine-derived amino acids may be present in a non-murine binding polypeptide, e.g., in an antigen binding site of a binding molecule. In therapeutic applications, preferred binding molecules to be used in the methods of the invention are not inmmunogenic in the animal to which the binding polypeptide is administered.

It will also be understood by one of ordinary skill in the art that the binding polypeptides for use in the diagnostic and treatment methods disclosed herein may be modified such that they vary in amino acid sequence from the naturally occurring binding polypeptide from which they were derived. For example, nucleotide or amino acid substitutions leading to conservative substitutions or changes at “non-essential” amino acid residues may be made.

In certain embodiments, a binding polypeptide for use in the methods of the invention comprises an amino acid sequence or one or more moieties not normally associated with that binding polypeptide. Exemplary modifications are described in more detail below. For example, a binding polypeptide of the invention may comprise a flexible linker sequence, or may be modified to add a functional moiety (e.g., PEG, a drug, a toxin, or a label).

A binding polypeptide for use in the methods of the invention may comprise, consist essentially of, or consist of a fusion protein. Fusion proteins are chimeric molecules which comprise a binding domain with at least one target binding site, and at least one heterologous portion.

A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a genotypically distinct entity from that of the rest of the entity to which it is being compared. For instance, a heterologous antigen may be derived from a different species origin, different cell type, or the same type of cell of distinct individuals.

The term “ligand binding domain” or “ligand binding portion” as used herein refers to any native receptor (e.g., cell surface receptor) or any region or derivative thereof retaining at least a qualitative ligand binding ability, and preferably the biological activity of a corresponding native receptor.

The term “receptor binding domain” or “receptor binding portion” as used herein refers to any native ligand or any region or derivative thereof retaining at least a qualitative receptor binding ability, and preferably the biological activity of a corresponding native ligand.

Antibody or Immunoglobulin. In one embodiment, the binding molecules for use in the diagnostic and treatment methods disclosed herein are “antibody” or “immunoglobulin” molecules, or immunospecific fragments thereof, e.g., naturally occurring antibody or immunoglobulin molecules or engineered antibody molecules or fragments that bind antigen in a manner similar to antibody molecules. The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises five broad classes of polypeptides that can be distinguished biochemically. All five classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region allows the antibody to selectively recognize and specifically bind epitopes on antigens. That is, the VL domain and VH domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In camelid species, however, the heavy chain variable region, referred to as VHH, forms the entire CDR. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH.

In one embodiment, an antigen binding molecule of the invention comprises at least one heavy or light chain CDR of an antibody molecule. In another embodiment, an antigen binding molecule of the invention comprises at least two CDRs from one or more antibody molecules. In another embodiment, an antigen binding molecule of the invention comprises at least three CDRs from one or more antibody molecules. In another embodiment, an antigen binding molecule of the invention comprises at least four CDRs from one or more antibody molecules. In another embodiment, an antigen binding molecule of the invention comprises at least five CDRs from one or more antibody molecules. In another embodiment, an antigen binding molecule of the invention comprises at least six CDRs from one or more antibody molecules. Exemplary antibody molecules comprising at least one CDR that can be included in the subject antigen binding molecules are known in the art and exemplary molecules are described herein.

Antibodies or immunospecific fragments thereof for use in the methods of the invention include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to binding molecules disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Antibodies or immunospecific fragments thereof for use in the diagnostic and therapeutic methods disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the invention comprises a polypeptide chain comprising a CH3 domain. Further, a binding polypeptide for use in the invention may lack at least a portion of a CH2 domain (e.g., all or part of a CH2 domain). As set forth above, it will be understood by one of ordinary skill in the art that these domains (e.g., the heavy chain portions) may be modified such that they vary in amino acid sequence from the naturally occurring immunoglobulin molecule.

In certain binding molecules, e.g., binding polypeptides, e.g., lung tumor-associated polypeptide-specific antibodies or immunospecific fragments thereof for use in the diagnostic and treatment methods disclosed herein, the heavy chain portions of one polypeptide chain of a multimer are identical to those on a second polypeptide chain of the multimer. Alternatively, heavy chain portion-containing monomers for use in the methods of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a V_{L} or C_{L} domain.

An isolated nucleic acid molecule encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into binding molecules for use in the diagnostic and treatment methods disclosed herein and screened for their ability to bind to the desired antigen, e.g., lung tumor-associated polypeptides and variants of fragments thereof.

Antibodies or fragment thereof for use in the diagnostic and therapeutic methods disclosed herein may be described or specified in terms of the epitope(s) or portion(s) of a target polypeptide that they recognize or specifically bind. The portion of an antigen which specifically interacts with the antigen binding domain of an antibody is an “epitope,” or an “antigenic determinant.” An antigen may comprise a single epitope, but typically, an antigen comprises at least two epitopes, and can include any number of epitopes, depending on the size, conformation, and type of antigen. Antigens are typically peptides or polypeptides, but can be any molecule or compound or a combination of molecules or compounds. For example, an organic compound, e.g., dinitrophenol or DNP, a nucleic acid, a carbohydrate, or a mixture of any of these compounds either with or without a peptide or polypeptide can be a suitable antigen. Thus, for example, an “epitope” on a polypeptide may include a carbohydrate side chain.

The minimum size of a peptide or polypeptide epitope is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide antigens preferably contain a sequence of at least 4, at least 5, at least 6, at least 7, more preferably at least 8, at least 9, at least 10, at least 15, at least 20, at least 25, and, most preferably, between about 15 to about 30 amino acids. Preferred peptides or polypeptides comprising, or alternatively consisting of, antigenic epitopes are at least 10, 15, 20, 25, 30, 35, 40, 45, 50, 55, 60, 65, 70, 75, 80, 85, 90, 95, or 100 amino acid residues in length.

By “specifically binds,” it is generally meant that an antibody binds to an epitope via its CDR, and that the binding entails some complementarity between the CDR and the epitope. According to this definition, an antibody is said to “specifically bind” to an epitope when it binds to that epitope, via its CDR more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody for use in the diagnostic and treatment methods disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

An antibody or fragment thereof for use in the diagnostic and treatment methods disclosed herein may be said to bind a target polypeptide disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Antibodies or immunospecific fragments thereof for use in the diagnostic and therapeutic methods disclosed herein may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

Antibodies or immunospecific fragments thereof for use in the diagnostic and treatment methods disclosed herein may also be described or specified in terms of their binding affinity to a polypeptide of the invention. Preferred binding affinities include those with a dissociation constant or Kd less than 5×10⁻²M, 10⁻²M, 5×10⁻³M, 10⁻³M, 5×10⁻⁴M, 10⁻⁴M, 5×10⁻⁵M, 10⁻⁵M, 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10hu −14M, 10⁻¹⁴M, 5×10⁻¹⁵M, or 10⁻¹⁵M.

Antibodies or immunospecific fragments thereof for use in the diagnostic and treatment methods disclosed herein may act as agonists or antagonists of target polypeptides described herein. For example, an antibody for use in the methods of the present invention may function as an antagonist, blocking or inhibiting the activity of the lung tumor-associated polypeptide.

As used herein, the term “binding site” or “binding domain” refers to a region of a binding molecule, e.g., a binding polypeptide, e.g., an antibody or fragment thereof, which is responsible for specifically binding to a target molecule of interest (e.g., an antigen, ligand, receptor, substrate or inhibitor) Exemplary binding domains include antibody variable domains, a receptor binding domain of a ligand, or a ligand binding domain of a receptor or an enzymatic domain. A binding domain on an antibody is referred to herein as an “antigen binding domain.”

A binding molecule, binding polypeptide, or antibody for use in the diagnostic and treatment methods disclosed herein may be “multispecific,” e.g., bispecific, trispecific or of greater multispecificity, meaning that it recognizes and binds to two or more different epitopes present on one or more different antigens (e.g., proteins) at the same time. Thus, whether a binding molecule is “monospecific” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in a binding molecule, binding polypeptide or antibody. Each binding domain specifically binds one epitope. When a binding molecule, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “V_{H} domain” includes the amino terminal variable domain of an immunoglobulin heavy chain and the term “C_{H}1 domain” includes the first (most amino terminal) constant region domain of an immunoglobulin heavy chain. The C_{H}1 domain is adjacent to the V_{H} domain and is amino terminal to the hinge region of an immunoglobulin heavy chain molecule.

As used herein the term “C_{H}2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat EA et al. op. cit. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues.

As used herein, the term “hinge region” includes the portion of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the C_{H}1 and C_{L} regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Antibodies of the present invention also include chimeric, single chain, and humanized antibodies.

As used herein the term “properly folded polypeptide” includes polypeptides (e.g., antigen binding molecules such as antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is a biochemical, expression includes the creation of that biochemical and any precursors.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as cancer, with a binding molecule which specifically binds a given target protein. As described in more detail herein, the binding molecule can be used in unconjugated form or can be conjugated, e.g., to a drug, prodrug, or an isotope.

By “hyperproliferative disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.”

Additional examples of hyperproliferative diseases, disorders, and/or conditions include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract.

Other hyperproliferative disorders include, but are not limited to: hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above.

As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The method of the present invention may be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)

Hyperplasia is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated by the method of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

### LUNG TUMOR ASSOCIATED POLYPEPTIDES

In certain embodiments, the present invention is directed to methods of treating or diagnosing hyperproliferative diseases such as cancer, comprising the use of binding molecules which specifically bind to lung tumor-associated proteins. These polypeptides were identified from the malignant tumor samples of patients with lung cancer, as described in the Examples herein, and are expressed at a level at least about 2.9 fold more relative to normal nonmalignant lung tissue. All polypeptides described herein were isolated from the membranes of tumor-associated cells. Thus, all lung tumor-associated proteins described herein are membrane proteins and contain at least one or all of the following domains: extracellular domain, transmembrane domain or intracellular domain.

Tables 1 and 2 list the lung tumor-associated polypeptides of the present invention which were isolated from the cellular membranes of tumors from human patients with lung cancer and were identified via mass spectroscopy analysis and sequence comparison to the Acembly 33 database using the Mascot® alignment program (Matrix Science Inc., Boston, Mass.), as described in Examples 1 and 2.

TABLE 1

| Seq | | | |
|---|---|---|---|
| ID | BI | | |
| NO: | Number | Protein Name | Description |
| 1 | BI6000000 | CKAP4.a | SIMILAR TO cytoskeleton-associated protein 4 (CKAP4) |
| 2 | BI6000001 | CUTL1.b | cut-like 1, CCAAT displacement protein (Drosophila) (CUTL1) |
| 3 | BI6000002 | DAD1.a | defender against cell death 1 (DAD1) |
| 4 | BI6000003 | DIA1.b | Homo sapiens gene DIA1 encoding diaphorase (NADH) |
| | | | (cytochrome b-5 reductase) |
| 5 | BI6000004 | EPHX1.c | SIMILAR TO epoxide hydrolase 1, microsomal (xenobiotic) |
| | | | (EPHX1) |
| 6 | BI6000005 | ITGB1.a | integrin, beta 1 (fibronectin receptor, beta polypeptide, antigen CD29 |
| | | | includes MDF2, MSK12) (ITGB1), transcript variant 1A |
| 7 | BI6000006 | Peptidase_M28.2.a | mRNA for KIAA1815 protein, partial cds. |
| 8 | BI6000007 | PTGFRN.a | mRNA for KIAA1436 protein, partial cds. |
| 9 | BI6000008 | STX4A.a | syntaxin 4A (placental) (STX4A) |
| 10 | BI6000009 | NP_002345 | tumor-associated calcium signal transducer 1 precursor [Homo |
| | | | sapiens] |
| 11 | BI6000010 | TMP21.b | transmembrane trafficking protein (TMP21) |
| 12 | BI6000011 | torira.a | putative protein family member, with a transmembrane domain, of |
| | | | eukaryotic origin |
| 13 | BI6000012 | | peptide of CKAP4.a |
| 14 | BI6000013 | | peptide of CUTL1.b |
| 15 | BI6000014 | | peptide of DAD1.a |
| 16 | BI6000015 | | peptide of DIA1.b |
| 17 | BI6000016 | | peptide of EPHX1.c |
| 18 | BI6000017 | | peptide of ITGB1.a |
| 19 | BI6000018 | | peptide of Peptidase_M28.2.a |
| 20 | BI6000019 | | peptide of PTGFRN.a |
| 21 | BI6000020 | | peptide of STX4A.a |
| 22 | BI6000021 | | peptide of NP_002345 |
| 23 | BI6000022 | | peptide of TMP21.b |
| 24 | BI6000023 | | peptide of torira.a |

TABLE 2

| | Protein | |
|---|---|---|
| SEQ ID NO: | Name | Description |
| 25 | ABHD1.a | abhydrolase domain containing 1 (ABHD1), transcript variant 1 |
| | | Hs.98608 e_val = 0 |
| 26 | ADAM11.a | a disintegrin and metalloproteinase domain 11 (ADAM 11), transcript |
| | | variant 1 Hs.6088 e_val = 0 |
| 27 | BAI1.a | brain-specific angiogenesis inhibitor 1 (BAI1) Hs.194654 e_val = 0 |
| 28 | FCER1G.b | Fc epsilon receptor, gamma chain |
| 29 | GPM6A.b | SIMILAR TO glycoprotein M6A (GPM6A) Hs.75819 e_val = 3.00E−123 |
| | | id = 86.45% coverage = 0.85 |
| 30 | GPR126.b | G protein-coupled receptor 126 (GPR126) Hs.44197 e_val = 0 |
| 31 | ITPR2.a | inositol 1,4,5-triphosphate receptor, type 2 |
| 32 | KCNMB1.b | SIMILAR TO potassium large conductance calcium-activated channel, |
| | | subfamily M, beta member 1 (KCNMB1) Hs.93841 e_val = 7.60E−56 |
| | | id = 100% coverage = 0.77 |
| 33 | L1CAM.a | L1 cell adhesion molecule (hydrocephalus, stenosis of aqueduct of |
| | | Sylvius 1, MASA (mental retardation, aphasia, shuffling gait and |
| | | adducted thumbs) syndrome, spastic paraplegia 1) (L1CAM), transcript |
| | | variant 1 Hs.1757 e_val = 0 |
| 34 | lorra.h | esophageal cancer associated protein (MGC16824) Hs.5320 |
| | | e_val = 9.00E−141 |
| 35 | LRIG1.a | leucine-rich repeats and immunoglobulin-like domains 1 (LRIG1) |
| | | Hs.4193 e_val = 0 |
| 36 | MRPS5.b | hypothetical protein FLJ14457 (FLJ14457) Hs.274414 e_val = 0 |
| 37 | MUC4.a | mucin 4 |
| 38 | nano.h | wi02a10.x1 NCI_CGAP_CLL1 Homo sapiens cDNA clone |
| | | IMAGE: 2389050 3′ Hs.369644 e_val = 4.00E−63 |
| 39 | NLGN4Y.a | KIAA0951 protein (KIAA0951) Hs.446306 e_val = 0 |
| 40 | NRP2.a | neuropilin 2 (NRP2) Hs.17778 e_val = 0 |
| 41 | NRXN2.a | neurexin 2 (NRXN2), transcript variant alpha-1 Hs.124085 e_val = 0 |
| 42 | PAM.g | KIAA0916 protein (KIAA0916) Hs.151411 e_val = 0 |
| 43 | R32184_3.a | SIMILAR TO hypothetical protein MGC4022 (R32184_3) Hs.380962 |
| | | e_val = 0 id = 98.54% coverage = 0.64 |
| 44 | SCUBE2.a | signal peptide, CUB domain, EGF-like 2 (SCUBE2) Hs.222399 e_val = 0 |
| 45 | SLC30A5.e | solute carrier family 30 (zinc transporter), member 5 (SLC30A5) |
| | | Hs.129445 e_val = 0 |
| 46 | SLC9A7.a | solute carrier family 9 (sodium/hydrogen exchanger), isoform 7 |
| | | (SLC9A7) Hs.154353 e_val = 0 |
| 47 | SORT1.a | sortilin 1 (SORT1) Hs.35 1872 e_val = 0 |
| 48 | TA-LRRP.a | T-cell activation leucine repeat-rich protein |
| 49 | TLR4.a | SIMILAR TO toll-like receptor 4 (TLR4), transcript variant 3 Hs.159239 |
| | | e_val = 0 id = 95.72% coverage = 1.02 |
| 50 | TTYH2.a | tweety homolog 2 (Drosophila) (TTYH2) Hs.27935 e_val = 0 |
| 51 | yasera.i | Human mRNA for KIAA0217 gene, partial cds. Hs.78851 e_val = 0 |
| 52 | zf- | hypothetical protein LOC285533 |
| | C3HC4.12.a | |

Table 3 lists the number of tumors that certain lung-associated polypeptides were detected in and the number of tumors that the lung associated-polypeptides were over-expressed at least 2.9 fold more in malignant samples relative to nonmalignant samples, as described in Example 3.

TABLE 3

| | # of Tumors in | # of Tumors |
|---|---|---|
| | which protein | which |
| | was >2.9 fold | expressed |
| SEQ ID NO: | over-expressed | protein |
| 25 | 2 | 2 |
| 26 | 2 | 2 |
| 27 | 2 | 2 |
| 28 | 2 | 4 |
| 29 | 2 | 2 |
| 30 | 2 | 3 |
| 31 | 2 | 2 |
| 32 | 2 | 2 |
| 33 | 3 | 3 |
| 34 | 2 | 2 |
| 35 | 3 | 3 |
| 36 | 2 | 2 |
| 37 | 1 | 1 |
| 38 | 2 | 2 |
| 39 | 2 | 2 |
| 40 | 2 | 2 |
| 41 | 2 | 2 |
| 42 | 2 | 3 |
| 43 | 2 | 2 |
| 44 | 2 | 2 |
| 45 | 2 | 2 |
| 46 | 2 | 2 |
| 47 | 2 | 2 |
| 48 | 2 | 2 |
| 49 | 2 | 2 |
| 50 | 2 | 2 |
| 51 | 2 | 2 |
| 52 | 2 | 2 |

In certain embodiments of the present invention antibodies are employed which recognize variant polypeptides or fragments thereof of the lung tumor-associated polypeptides described herein. In certain embodiments variant polypeptides, or fragments thereof, of the lung tumor-associated polypeptides include a predicted domain or region of the lung tumor-associated polypeptides described herein. In certain embodiments, binding molecules such as antibodies which bind variant polypeptides, and fragments thereof, of the extracellular domains of the lung tumor-associated polypeptides are employed.

Domains of certain lung tumor-associated polypeptides have been predicted based on homology to known polypeptide domains using the pfam program (see Bateman, A., et al., Nucl. Acids Res., 2004, Vol. 32, Database Issue, D138-D141). Table 4 below describes exemplary fragments based on homologies to known domains and the amino acid sequence positions which define the approximate beginning and end of the domains. The same method can be used to predict the domains of all lung-tumor associated polypeptides described herein.

TABLE 4

| | From AA | To AA | |
|---|---|---|---|
| Protein | Position | Position | Domain Description |
| SEQ ID NO: 25 | 58 | 80 | Arterivirus glycoprotein |
| SEQ ID NO: 25 | 137 | 369 | Putative esterase |
| SEQ ID NO: 25 | 158 | 277 | Copper type II ascorbate-dependent monooxygenase, N- |
| | | | terminal domain |
| SEQ ID NO: 25 | 173 | 299 | Cytochrome C biogenesis protein |
| SEQ ID NO: 25 | 176 | 422 | Thioesterase domain |
| SEQ ID NO: 25 | 205 | 283 | alpha/beta hydrolase fold |
| SEQ ID NO: 25 | 402 | 424 | Hydrogenase-1 expression protein HyaE |
| SEQ ID NO: 26 | 100 | 216 | Reprolysin family propeptide |
| SEQ ID NO: 26 | 239 | 438 | Reprolysin (M12B) family zinc metalloprotease |
| SEQ ID NO: 26 | 453 | 529 | Disintegrin |
| SEQ ID NO: 26 | 677 | 709 | EGF-like domain |
| SEQ ID NO: 27 | 65 | 88 | Mucin-like glycoprotein |
| SEQ ID NO: 27 | 289 | 297 | Protein of unknown function, DUF645 |
| SEQ ID NO: 27 | 321 | 370 | Thrombospondin type 1 domain |
| SEQ ID NO: 27 | 414 | 462 | Thrombospondin type 1 domain |
| SEQ ID NO: 27 | 418 | 429 | Nine Cysteines Domain of family 3 GPCR |
| SEQ ID NO: 27 | 469 | 517 | Thrombospondin type 1 domain |
| SEQ ID NO: 27 | 473 | 486 | Nine Cysteines Domain of family 3 GPCR |
| SEQ ID NO: 27 | 527 | 575 | Thrombospondin type 1 domain |
| SEQ ID NO: 27 | 533 | 549 | Nine Cysteines Domain of family 3 GPCR |
| SEQ ID NO: 27 | 582 | 630 | Thrombospondin type 1 domain |
| SEQ ID NO: 27 | 634 | 695 | Hormone receptor domain |
| SEQ ID NO: 27 | 936 | 994 | Latrophilin/CL-1-like GPS domain |
| SEQ ID NO: 27 | 982 | 1248 | C. elegans Srg family integral membrane protein |
| SEQ ID NO: 27 | 993 | 1339 | Slime mold cyclic AMP receptor |
| SEQ ID NO: 27 | 1000 | 1247 | 7 transmembrane receptor (Secretin family) |
| SEQ ID NO: 27 | 1005 | 1249 | 7 transmembrane receptor (metabotropic glutamate |
| | | | family) |
| SEQ ID NO: 27 | 1021 | 1243 | 7 transmembrane receptor (rhodopsin family) |
| SEQ ID NO: 27 | 1023 | 1240 | 7TM chemoreceptor |
| SEQ ID NO: 27 | 1302 | 1541 | Extensin-like region |
| SEQ ID NO: 28 | 75 | 95 | Immunoreceptor tyrosine-based activation motif |
| SEQ ID NO: 29 | 50 | 291 | Myelin proteolipid protein (PLP or lipophilin) |
| SEQ ID NO: 30 | 154 | 207 | Latrophilin/CL-1-like GPS domain |
| SEQ ID NO: 30 | 211 | 469 | 7TM chemoreceptor |
| SEQ ID NO: 30 | 216 | 476 | 7 transmembrane receptor (Secretin family) |
| SEQ ID NO: 30 | 229 | 419 | Cobalamin-5-phosphate synthase |
| SEQ ID NO: 30 | 235 | 475 | 7 transmembrane receptor (rhodopsin family) |
| SEQ ID NO: 30 | 253 | 455 | Mycoplasma MFS transporter |
| SEQ ID NO: 30 | 286 | 304 | Antenna complex alpha/beta subunit |
| SEQ ID NO: 30 | 291 | 482 | Binding-protein-dependent transport system inner |
| | | | membrane component |
| SEQ ID NO: 30 | 331 | 353 | BphX-like |
| SEQ ID NO: 30 | 485 | 496 | Uncharacterised ACR, COG2135 |
| SEQ ID NO: 31 | 50 | 104 | MIR domain |
| SEQ ID NO: 31 | 111 | 161 | MIR domain |
| SEQ ID NO: 31 | 169 | 225 | MIR domain |
| SEQ ID NO: 31 | 232 | 340 | MIR domain |
| SEQ ID NO: 31 | 409 | 615 | RIH domain |
| SEQ ID NO: 31 | 1119 | 1293 | RIH domain |
| SEQ ID NO: 31 | 2252 | 2478 | Ion transport protein |
| SEQ ID NO: 32 | 2 | 122 | Calcium-activated potassium channel, beta subunit |
| SEQ ID NO: 33 | 34 | 133 | Immunoglobulin V-set domain |
| SEQ ID NO: 33 | 35 | 133 | Immunoglobulin I-set domain |
| SEQ ID NO: 33 | 50 | 116 | Immunoglobulin domain |
| SEQ ID NO: 33 | 138 | 172 | Immunoglobulin V-set domain |
| SEQ ID NO: 33 | 144 | 159 | Immunoglobulin I-set domain |
| SEQ ID NO: 33 | 151 | 211 | Immunoglobulin domain |
| SEQ ID NO: 33 | 235 | 400 | Glycogen synthase kinase-3 binding |
| SEQ ID NO: 33 | 242 | 330 | Immunoglobulin I-set domain |
| SEQ ID NO: 33 | 242 | 330 | Immunoglobulin V-set domain |
| SEQ ID NO: 33 | 249 | 320 | Immunoglobulin C1-set domain |
| SEQ ID NO: 33 | 257 | 314 | Immunoglobulin domain |
| SEQ ID NO: 33 | 293 | 307 | Cytochrome c oxidase subunit VIa |
| SEQ ID NO: 33 | 333 | 422 | Immunoglobulin I-set domain |
| SEQ ID NO: 33 | 347 | 406 | Immunoglobulin domain |
| SEQ ID NO: 33 | 426 | 515 | Immunoglobulin I-set domain |
| SEQ ID NO: 33 | 441 | 499 | Immunoglobulin domain |
| SEQ ID NO: 33 | 471 | 511 | Immunoglobulin V-set domain |
| SEQ ID NO: 33 | 517 | 609 | Immunoglobulin V-set domain |
| SEQ ID NO: 33 | 518 | 609 | Immunoglobulin I-set domain |
| SEQ ID NO: 33 | 532 | 593 | Immunoglobulin domain |
| SEQ ID NO: 33 | 612 | 701 | Fibronectin type III domain |
| SEQ ID NO: 33 | 714 | 800 | Fibronectin type III domain |
| SEQ ID NO: 33 | 743 | 763 | Domain of unknown function (DUF317) |
| SEQ ID NO: 33 | 812 | 907 | Fibronectin type III domain |
| SEQ ID NO: 33 | 918 | 1005 | Fibronectin type III domain |
| SEQ ID NO: 33 | 1017 | 1097 | Fibronectin type III domain |
| SEQ ID NO: 33 | 1130 | 1148 | Basic membrane protein |
| SEQ ID NO: 34 | 34 | 67 | Tetratricopeptide repeat |
| SEQ ID NO: 35 | 234 | 261 | Leucine rich repeat N-terminal domain |
| SEQ ID NO: 35 | 263 | 286 | Leucine Rich Repeat |
| SEQ ID NO: 35 | 287 | 308 | Leucine Rich Repeat |
| SEQ ID NO: 35 | 310 | 330 | Leucine Rich Repeat |
| SEQ ID NO: 35 | 334 | 357 | Leucine Rich Repeat |
| SEQ ID NO: 35 | 358 | 381 | Leucine Rich Repeat |
| SEQ ID NO: 35 | 383 | 405 | Leucine Rich Repeat |
| SEQ ID NO: 35 | 406 | 429 | Leucine Rich Repeat |
| SEQ ID NO: 35 | 430 | 453 | Leucine Rich Repeat |
| SEQ ID NO: 35 | 454 | 477 | Leucine Rich Repeat |
| SEQ ID NO: 35 | 478 | 501 | Leucine Rich Repeat |
| SEQ ID NO: 35 | 502 | 528 | Leucine Rich Repeat |
| SEQ ID NO: 35 | 502 | 525 | Leucine Rich Repeat |
| SEQ ID NO: 35 | 526 | 549 | Leucine Rich Repeat |
| SEQ ID NO: 35 | 539 | 552 | Imidazoleglycerol-phosphate dehydratase |
| SEQ ID NO: 35 | 550 | 573 | Leucine Rich Repeat |
| SEQ ID NO: 35 | 550 | 563 | Leucine Rich Repeat |
| SEQ ID NO: 35 | 577 | 600 | Leucine Rich Repeat |
| SEQ ID NO: 35 | 601 | 624 | Leucine Rich Repeat |
| SEQ ID NO: 35 | 625 | 645 | Leucine Rich Repeat |
| SEQ ID NO: 35 | 625 | 654 | Leucine Rich Repeat |
| SEQ ID NO: 35 | 659 | 684 | Leucine rich repeat C-terminal domain |
| SEQ ID NO: 35 | 689 | 790 | Immunoglobulin I-set domain |
| SEQ ID NO: 35 | 689 | 790 | Immunoglobulin V-set domain |
| SEQ ID NO: 35 | 703 | 773 | Immunoglobulin domain |
| SEQ ID NO: 35 | 793 | 884 | Immunoglobulin I-set domain |
| SEQ ID NO: 35 | 793 | 884 | Immunoglobulin V-set domain |
| SEQ ID NO: 35 | 807 | 868 | Immunoglobulin domain |
| SEQ ID NO: 35 | 867 | 883 | PKD domain |
| SEQ ID NO: 35 | 887 | 975 | Immunoglobulin I-set domain |
| SEQ ID NO: 35 | 887 | 975 | Immunoglobulin V-set domain |
| SEQ ID NO: 35 | 897 | 974 | Adenovirus E3 region protein CR1 |
| SEQ ID NO: 35 | 901 | 959 | Immunoglobulin domain |
| SEQ ID NO: 36 | 74 | 114 | KRAB box |
| SEQ ID NO: 36 | 274 | 286 | XPA protein N-terminal |
| SEQ ID NO: 36 | 277 | 299 | Zinc finger, C2H2 type |
| SEQ ID NO: 36 | 277 | 294 | Zinc knuckle |
| SEQ ID NO: 36 | 277 | 287 | Transcription factor S-II (TFIIS) |
| SEQ ID NO: 36 | 302 | 314 | XPA protein N-terminal |
| SEQ ID NO: 36 | 305 | 327 | Zinc finger, C2H2 type |
| SEQ ID NO: 36 | 305 | 315 | Transcription factor S-II (TFIIS) |
| SEQ ID NO: 36 | 307 | 348 | GATA zinc finger |
| SEQ ID NO: 36 | 330 | 342 | XPA protein N-terminal |
| SEQ ID NO: 36 | 332 | 340 | Domain of unknown function (DUF1610) |
| SEQ ID NO: 36 | 333 | 355 | Zinc finger, C2H2 type |
| SEQ ID NO: 36 | 333 | 343 | Transcription factor S-II (TFIIS) |
| SEQ ID NO: 36 | 335 | 356 | Transposase |
| SEQ ID NO: 36 | 358 | 370 | XPA protein N-terminal |
| SEQ ID NO: 36 | 360 | 368 | Domain of unknown function (DUF1610) |
| SEQ ID NO: 36 | 361 | 383 | Zinc finger, C2H2 type |
| SEQ ID NO: 36 | 361 | 371 | Transcription factor S-II (TFIIS) |
| SEQ ID NO: 36 | 386 | 398 | XPA protein N-terminal |
| SEQ ID NO: 36 | 389 | 411 | Zinc finger, C2H2 type |
| SEQ ID NO: 36 | 389 | 399 | Transcription factor S-II (TFIIS) |
| SEQ ID NO: 36 | 390 | 412 | BED zinc finger |
| SEQ ID NO: 36 | 391 | 412 | Transposase |
| SEQ ID NO: 36 | 402 | 440 | BED zinc finger |
| SEQ ID NO: 36 | 414 | 426 | XPA protein N-terminal |
| SEQ ID NO: 36 | 416 | 424 | Domain of unknown function (DUF1610) |
| SEQ ID NO: 36 | 417 | 439 | Zinc finger, C2H2 type |
| SEQ ID NO: 36 | 417 | 427 | Transcription factor S-II (TFIIS) |
| SEQ ID NO: 36 | 417 | 440 | Transposase |
| SEQ ID NO: 36 | 442 | 455 | XPA protein N-terminal |
| SEQ ID NO: 36 | 444 | 452 | Domain of unknown function (DUF1610) |
| SEQ ID NO: 36 | 445 | 467 | Zinc finger, C2H2 type |
| SEQ ID NO: 36 | 445 | 455 | Transcription factor S-II (TFIIS) |
| SEQ ID NO: 37 | 719 | 727 | Hepatitis core antigen |
| SEQ ID NO: 37 | 1255 | 1340 | Nidogen-like |
| SEQ ID NO: 37 | 1341 | 1456 | AMOP domain |
| SEQ ID NO: 37 | 1362 | 1389 | Plant specific eukaryotic initiation factor 4B |
| SEQ ID NO: 37 | 1470 | 1642 | von Willebrand factor type D domain |
| SEQ ID NO: 37 | 1476 | 1487 | Coronavirus non-structural protein NS4 |
| SEQ ID NO: 37 | 1644 | 1662 | Transketolase, C-terminal domain |
| SEQ ID NO: 37 | 1675 | 1684 | Peptidase family M1 |
| SEQ ID NO: 37 | 1824 | 1859 | EGF-like domain |
| SEQ ID NO: 37 | 1885 | 1896 | EGF-like domain |
| SEQ ID NO: 37 | 1910 | 1944 | EGF-like domain |
| SEQ ID NO: 37 | 1928 | 1943 | Leucine rich repeat N-terminal domain |
| SEQ ID NO: 37 | 2112 | 2147 | EGF-like domain |
| SEQ ID NO: 37 | 2121 | 2161 | TB domain |
| SEQ ID NO: 39 | 1 | 422 | Carboxylesterase |
| SEQ ID NO: 39 | 77 | 87 | NAD-dependent glycerol-3-phosphate dehydrogenase N- |
| | | | terminus |
| SEQ ID NO: 39 | 309 | 322 | Hantavirus glycoprotein G2 |
| SEQ ID NO: 39 | 507 | 537 | Bacteriorhodopsin |
| SEQ ID NO: 39 | 519 | 538 | Picornaviridae P3A protein |
| SEQ ID NO: 39 | 591 | 602 | Phage P2 GpU |
| SEQ ID NO: 40 | 1 | 16 | PetN |
| SEQ ID NO: 40 | 28 | 139 | CUB domain |
| SEQ ID NO: 40 | 121 | 141 | F5/8 type C domain |
| SEQ ID NO: 40 | 149 | 264 | CUB domain |
| SEQ ID NO: 40 | 292 | 424 | F5/8 type C domain |
| SEQ ID NO: 40 | 449 | 589 | F5/8 type C domain |
| SEQ ID NO: 40 | 646 | 802 | MAM domain |
| SEQ ID NO: 41 | 141 | 274 | Laminin G domain |
| SEQ ID NO: 41 | 141 | 271 | Laminin G domain |
| SEQ ID NO: 41 | 290 | 325 | EGF-like domain |
| SEQ ID NO: 41 | 402 | 546 | Laminin G domain |
| SEQ ID NO: 41 | 402 | 543 | Laminin G domain |
| SEQ ID NO: 41 | 605 | 753 | Laminin G domain |
| SEQ ID NO: 41 | 605 | 750 | Laminin G domain |
| SEQ ID NO: 41 | 778 | 800 | EGF-like domain |
| SEQ ID NO: 41 | 844 | 974 | Laminin G domain |
| SEQ ID NO: 41 | 890 | 925 | Laminin G domain |
| SEQ ID NO: 41 | 1030 | 1161 | Laminin G domain |
| SEQ ID NO: 41 | 1030 | 1158 | Laminin G domain |
| SEQ ID NO: 41 | 1184 | 1216 | EGF-like domain |
| SEQ ID NO: 41 | 1253 | 1402 | Laminin G domain |
| SEQ ID NO: 42 | 129 | 157 | Hepatocyte nuclear factor 1 (HNF-1), alpha isoform C |
| | | | terminus |
| SEQ ID NO: 42 | 647 | 655 | XPA protein N-terminal |
| SEQ ID NO: 43 | 39 | 49 | Cytochrome c/c1 heme lyase |
| SEQ ID NO: 43 | 74 | 91 | Choristoneura fumiferana antifreeze protein (CfAFP) |
| SEQ ID NO: 43 | 264 | 281 | Viral matrix protein |
| SEQ ID NO: 43 | 356 | 372 | Carotene hydroxylase |
| SEQ ID NO: 44 | 71 | 110 | Calcium binding EGF domain |
| SEQ ID NO: 44 | 75 | 110 | EGF-like domain |
| SEQ ID NO: 44 | 158 | 193 | EGF-like domain |
| SEQ ID NO: 44 | 203 | 239 | EGF-like domain |
| SEQ ID NO: 44 | 312 | 347 | EGF-like domain |
| SEQ ID NO: 44 | 349 | 388 | Calcium binding EGF domain |
| SEQ ID NO: 44 | 390 | 427 | Calcium binding EGF domain |
| SEQ ID NO: 44 | 429 | 468 | Calcium binding EGF domain |
| SEQ ID NO: 44 | 433 | 468 | EGF-like domain |
| SEQ ID NO: 44 | 670 | 720 | GCC2 and GCC3 |
| SEQ ID NO: 44 | 727 | 774 | GCC2 and GCC3 |
| SEQ ID NO: 44 | 783 | 830 | GCC2 and GCC3 |
| SEQ ID NO: 44 | 835 | 944 | CUB domain |
| SEQ ID NO: 45 | 4 | 516 | Major Facilitator Superfamily |
| SEQ ID NO: 45 | 21 | 199 | Cytochrome b561 |
| SEQ ID NO: 45 | 37 | 418 | Bacterial Cytochrome Ubiquinol Oxidase |
| SEQ ID NO: 45 | 101 | 115 | Geminivirus coat protein |
| SEQ ID NO: 45 | 110 | 268 | NnrU protein |
| SEQ ID NO: 45 | 320 | 534 | Sec-independent protein translocase protein (TatC) |
| SEQ ID NO: 45 | 321 | 596 | Cation efflux family |
| SEQ ID NO: 45 | 353 | 591 | High-affinity nickel-transport protein |
| SEQ ID NO: 45 | 438 | 456 | Small secreted domain (DUF320) |
| SEQ ID NO: 46 | 26 | 50 | BphX-like |
| SEQ ID NO: 46 | 35 | 457 | MviN-like protein |
| SEQ ID NO: 46 | 62 | 468 | Permease for cytosine/purines, uracil, thiamine, allantoin |
| SEQ ID NO: 46 | 74 | 534 | Sodium/hydrogen exchanger family |
| SEQ ID NO: 46 | 324 | 496 | Bacitracin resistance protein BacA |
| SEQ ID NO: 46 | 364 | 378 | Protein of unknown function (DUF1218) |
| SEQ ID NO: 46 | 441 | 470 | Protein of unknown function (DUF1200) |
| SEQ ID NO: 46 | 552 | 565 | Viral Beta C/D like family |
| SEQ ID NO: 47 | 145 | 156 | BNR/Asp-box repeat |
| SEQ ID NO: 47 | 240 | 251 | BNR/Asp-box repeat |
| SEQ ID NO: 47 | 287 | 298 | BNR/Asp-box repeat |
| SEQ ID NO: 47 | 328 | 339 | BNR/Asp-box repeat |
| SEQ ID NO: 47 | 377 | 388 | BNR/Asp-box repeat |
| SEQ ID NO: 47 | 428 | 439 | BNR/Asp-box repeat |
| SEQ ID NO: 47 | 506 | 517 | BNR/Asp-box repeat |
| SEQ ID NO: 47 | 548 | 559 | BNR/Asp-box repeat |
| SEQ ID NO: 48 | 506 | 528 | Leucine Rich Repeat |
| SEQ ID NO: 48 | 529 | 556 | Leucine Rich Repeat |
| SEQ ID NO: 48 | 604 | 626 | Leucine Rich Repeat |
| SEQ ID NO: 48 | 627 | 651 | Leucine Rich Repeat |
| SEQ ID NO: 48 | 652 | 674 | Leucine Rich Repeat |
| SEQ ID NO: 48 | 675 | 697 | Leucine Rich Repeat |
| SEQ ID NO: 48 | 698 | 720 | Leucine Rich Repeat |
| SEQ ID NO: 48 | 721 | 743 | Leucine Rich Repeat |
| SEQ ID NO: 48 | 744 | 766 | Leucine Rich Repeat |
| SEQ ID NO: 48 | 767 | 789 | Leucine Rich Repeat |
| SEQ ID NO: 49 | 51 | 95 | Anenome neurotoxin |
| SEQ ID NO: 49 | 110 | 133 | Leucine Rich Repeat |
| SEQ ID NO: 49 | 134 | 157 | Leucine Rich Repeat |
| SEQ ID NO: 49 | 158 | 181 | Leucine Rich Repeat |
| SEQ ID NO: 49 | 182 | 205 | Leucine Rich Repeat |
| SEQ ID NO: 49 | 206 | 230 | Leucine Rich Repeat |
| SEQ ID NO: 49 | 231 | 254 | Leucine Rich Repeat |
| SEQ ID NO: 49 | 244 | 261 | Leucine rich repeat C-terminal domain |
| SEQ ID NO: 49 | 371 | 381 | Protein of unknown function (DUF1426) |
| SEQ ID NO: 49 | 388 | 409 | Leucine Rich Repeat |
| SEQ ID NO: 49 | 429 | 446 | Leucine Rich Repeat |
| SEQ ID NO: 49 | 455 | 477 | Leucine Rich Repeat |
| SEQ ID NO: 49 | 478 | 499 | Leucine Rich Repeat |
| SEQ ID NO: 49 | 503 | 526 | Leucine Rich Repeat |
| SEQ ID NO: 49 | 527 | 551 | Leucine Rich Repeat |
| SEQ ID NO: 49 | 541 | 556 | Protein of unknown function (DUF1280) |
| SEQ ID NO: 49 | 552 | 575 | Leucine Rich Repeat |
| SEQ ID NO: 49 | 576 | 599 | Leucine Rich Repeat |
| SEQ ID NO: 49 | 576 | 602 | Leucine Rich Repeat |
| SEQ ID NO: 49 | 600 | 630 | Leucine Rich Repeat |
| SEQ ID NO: 49 | 661 | 683 | Leucine rich repeat C-terminal domain |
| SEQ ID NO: 49 | 684 | 704 | Cell surface immobilisation antigen SerH |
| SEQ ID NO: 49 | 731 | 869 | TIR domain |
| SEQ ID NO: 50 | 27 | 433 | Tweety |
| SEQ ID NO: 52 | 104 | 154 | Protein of unknown function (DUF1147) |
| SEQ ID NO: 52 | 174 | 186 | Protein of unknown function (DUF728) |
| SEQ ID NO: 52 | 226 | 279 | PHD-finger |
| SEQ ID NO: 52 | 227 | 276 | Zinc finger, C3HC4 type (RING finger) |
| SEQ ID NO: 52 | 227 | 233 | AN1-like Zinc finger |
| SEQ ID NO: 52 | 273 | 282 | AN1-like Zinc finger |

Intracellular, extracellular and nontransmembrane domains of certain lung-tumor associated polypeptides were predicted by the Kyte and Doolittle hydropathy algorithm (Kyte, J. and Doolittle, R., J. Mol. Biol. 157: 105-132 (1982)), Chou and Fasman method to predict secondary structure (Chou and Fasman, Adv. Enz.:47, 45-147 (1978), and Goldman, Engelman and Steitz Transbilayer Helices Prediction algorithm (Engelman, D. M. et al. Annu. Rev. Biophys. Biophys. Chem. 15:321-353 (1986)). The same method can be used to predict intracellular, extracellular and nontransmembrane domains of all lung-tumor associated polypeptides decribed herein.

Table 5 below provides portions of each lung tumor-associated polypeptide which are predicted to be part of the intracellular, extracellular and nontransmembrane portions of the polypeptide.

TABLE 5

| | Predicted | Predicted | |
|---|---|---|---|
| | Intracellular | Extracellular | Predicted Non- |
| SEQ ID NO | Regions | Regions | Transmembrane Regions |
| 25 | 1-69 | 90-458 | 1-69, 90-458 |
| 26 | 759-769 | 1-735 | 1-735, 759-769 |
| 27 | 1027-1037, 1088-1108, | 1-1003, 1061-1064, | 1-1003, 1027-1037, 1061-1064, |
| | 1171-1190 | 1129-1147, | 1088-1108, 1129-1147, 1171-1190, |
| | | 1214-1640 | 1214-1640 |
| 28 | 1-12, 60-99 | 33-36 | 1-12, 33-36, 60-99 |
| 29 | 1-57, 136-147, 197-247 | 81-112, 171-173, 271-315 | 1-57, 81-112, 136-147, 171-173, |
| | | | 197-247, 271-315 |
| 30 | 243-254, 306-324, | 1-219, 278-282, 348-380, | 1-219, 243-254, 278-282, 306-324, |
| | 404-423, 473-576 | 447-449 | 348-380, 404-423, 447-449, |
| | | | 473-576 |
| 31 | 2187-2197, 2263-2282, | 1-2163, 2221-2239, | 1-2163, 2187-2197, 2221-2239, |
| | 2356-2459 | 2306-2332, | 2263-2282, 2306-2332, 2356-2459, |
| | | 2483-2639 | 2483-2639 |
| 32 | 40-130 | 40-130 | 1-16, 40-130 |
| 33 | 1144-1257 | 1-1120 | 1-1120, 1144-1257 |
| 34 | 1-11 | 35-248 | 1-11, 35-248 |
| 35 | 1010-1287 | 1-986 | 1-986, 1010-1287 |
| 36 | 1-4 | 28-473 | 1-4, 28-473 |
| 37 | 2183-2200 | 1-2159 | 1-2159, 2183-2200 |
| 38 | 143-153 | 1-119 | 1-119, 143-153 |
| 39 | 530-648 | 1-506 | 1-506, 530-648 |
| 40 | 891-931 | 1-867 | 1-867, 891-931 |
| 41 | 1743-1796 | 1-1719 | 1-1719, 1743-1796 |
| 42 | 1-29 | 53-693 | 1-29, 53-693 |
| 43 | 90-307, 370-375, 427-432 | 1-66, 331-349, 394-407, | 1-66, 90-307, 331-349, 370-375, |
| | | 456-626 | 394-407, 427-432, 456-626 |
| 44 | 1-35 | 59-1025 | 1-35, 59-1025 |
| 45 | 43-53, 119-138, 191-202, | 1-19, 69-95, 159-167, | 1-19, 43-53, 69-95, 119-138, 159-167, |
| | 265-320, 374-385, | 226-244, 342-350, | 191-202, 226-244, 265-320, |
| | 446-492, 543-596 | 409-422, 516-519 | 342-350, 374-385, 409-422, |
| | | | 446-492, 516-519, 543-596 |
| 46 | 92-97, 200-211, 272-282, | 1-71, 121-181, 235-248, | 1-71, 92-97, 121-181, 200-211, 235-248, |
| | 348-371, 434-437, | 306-324, 395-413, | 272-282, 306-324, 348-371, |
| | 498-509 | 461-474, 533-725 | 395-413, 434-437, 461-474, |
| | | | 498-509, 533-725 |
| 47 | 779-831 | 1-755 | 1-755, 779-831 |
| 48 | 64-137, 303-322 | 1-43, 161-279, 346-821 | 1-43, 64-137, 161-279, 303-322, |
| | | | 346-821 |
| 49 | 712-894 | 1-688 | 1-688, 712-894 |
| 50 | 1-11, 68-86, 236-239, | 31-44, 110-212, 263-387 | 1-11, 31-44, 68-86, 110-212, 236-239, |
| | 411-534 | | 263-387, 411-534 |
| 51 | 1-6 | 27-54 | 1-6, 27-54 |
| 52 | 74-79, 122-139, 195-302 | 1-50, 99-101, 163-176, | 1-50, 74-79, 99-101, 122-139, 163-176, |
| | | 326-328 | 195-302, 326-328 |

In the context of the amino acids comprising the various structural and functional domains of a lung tumor-associated polypeptide, the term “about” includes the particularly recited value and values larger or smaller by several (e.g., 10, 9, 8, 7, 6, 5, 4, 3, 2, or 1) amino acids. One of ordinary skill would appreciate that the amino acid residues constituting these domains may vary slightly (e.g., by about 1 to 15 residues) depending on the criteria used to define the domain. Thus in various embodiments, the extracellular domain of a lung associated polypeptide comprises, consists essentially of, or consists of, for example, the amino acid residues listed in Table 5 as comprising the extracellular domain.

### TREATMENT METHODS USING THERAPEUTIC BINDING MOLECULES, IN PARTICULAR, LUNG TUMOR-ASSOCIATED-SPECIFIC ANTIBODIES, OR IMMUNOSPECIFIC FRAGMENTS THEREOF

One embodiment of the present invention provides methods for treating a hyperproliferative disease or disorder, e.g., cancer, a malignancy, a tumor, or a metastasis thereof, in an animal suffering from such disease or predisposed to contract such disease, the method comprising, consisting essentially of, or consisting of administering to the animal an effective amount of a binding molecule, more specifically a binding polypeptide, and even more specifically an antibody or immunospecific fragment thereof, that binds to a lung tumor-associated polypeptide described herein. A specific embodiment of the present invention is a method of treatment as above, where the binding molecule binds specifically to at least one epitope of a polypeptide selected from the group consisting of SEQ ID NOs: 1 to 52.

A therapeutic binding molecule, e.g., a binding polypeptide, e.g., an antibody that binds specifically to a lung tumor-associated polypeptide described herein, to be used in treatment methods disclosed herein can be prepared and used as a therapeutic agent that stops, reduces, prevents, or inhibits cellular activities involved in cellular hyperproliferation, e.g., cellular activities that induce the altered or abnormal pattern of vascularization that is often associated with hyperproliferative diseases or disorders. Characteristics of lung tumor-associated proteins that are suitable targets for such binding molecules include lung tumor-associated polypeptides located on the cell surface and disease- or disorder-specific expression; e.g., by cells of tumor-induced or inflammatory vascular tissue. Therapeutic binding molecules that bind specifically to such disease- or disorder-associated proteins are referred to herein as binding molecules or binding polypeptides. In certain embodiments, the binding molecule has at least one binding domain which specifically binds to a target molecule such as a polypeptide, e.g., a tumor-expressed or tumor-associated cell surface antigen.

Binding polypeptides include antibodies or immunospecific fragments thereof such as monoclonal, chimeric or humanized antibodies, and fragments of antibodies that bind specifically to lung tumor-associated proteins. The antibodies may be monovalent, bivalent, polyvalent, or bifunctional antibodies, and the antibody fragments include Fab F(ab′)₂, and Fv. Therapeutic binding molecules produced according to the invention also include fusion proteins that target a ligand or receptor of a lung tumor-associated polypeptide described herein which is expressed on the surface of a disease-associated cell. Another type of binding polypeptide, also used herein as an immunogen, comprises a non-antigen-specific fragment of an immunoglobulin joined to the extracellular domain of a transmembrane lung tumor-associated protein to generate a receptor: Ig fusion protein that antagonizes and neutralizes the cellular function of the target protein.

Therapeutic binding molecules according to the invention can be used in unlabeled or unconjugated form, or can be coupled or linked to cytotoxic moieties such as radiolabels and biochemical cytotoxins to produce agents that exert therapeutic effects.

In certain embodiments, a binding domain on a binding molecule or binding polypeptide is an antigen binding domain, and the binding polypeptide is an antibody, or immunospecific fragment thereof. An antigen binding domain is formed by antibody variable regions that vary from one antibody to another. Naturally occurring antibodies comprise at least two antigen binding domains, i.e., they are at least bivalent. As used herein, the term “antigen binding domain” includes a site that specifically binds an epitope on an antigen (e.g., a cell surface or soluble antigen). The antigen binding domain of an antibody typically includes at least a portion of an immunoglobulin heavy chain variable region and at least a portion of an immunoglobulin light chain variable region. The binding site formed by these variable regions determines the specificity of the antibody.

While a lung-tumor associated polypeptide described herein can be expressed in disease- or disorder-associated tissue, the therapeutic agent that binds the targeted protein can also exert a therapeutic effect by binding to the targeted protein present on non-vascular tissues associated with the disease or disorder. For example, the invention includes methods for inhibiting tumor angiogenesis and growth in a mammal comprising administering a binding agent that binds specifically to a vascular protein identified by the invention as being specifically present in tumor-associated tissue. The vascular protein identified by the invention as being specifically present in tumor-associated tissue may also be expressed by the tumor tissue itself, so that in addition to inhibiting tumor angiogenesis through binding to the targeted protein in the tumor vasculature, an anti-tumor agent, e.g., a binding molecule, that binds specifically to the targeted protein according to the invention might also inhibit growth of a tumor by binding to and killing tumor cells directly, or by blocking invasiveness of tumor cells.

The present invention provides methods for treating various hyperproliferative disorders, e.g., by inhibiting tumor growth, in a mammal, comprising, consisting essentially of, or consisting of administering to the mammal an effective amount of a binding agent that binds specifically to a transmembrane vascular protein identified by the invention as being specifically or predominantly present in lung tumor cells or lung tumor-associated tissue.

In addition to antibodies and immunospecific fragments thereof, binding molecules of the present invention include a fusion protein, an agent which elicits a T-cell response specific for the lung tumor-associated polypeptides, variants or fragments described herein, and a small molecule. Similar binding molecules may be used in the in vitro and in vivo diagnostic methods described in more detail below.

The present invention is more specifically directed to a method of treating a hyperproliferative disease, e.g., inhibiting or preventing tumor formation, tumor growth, tumor invasiveness, and/or metastasis formation, in an animal, e.g., a mammal, e.g., a human, comprising, consisting essentially of, or consisting of administering to an animal in need thereof an effective amount of a binding agent, e.g., a binding molecule, more specifically a binding polypeptide, and even more specifically an antibody or immunospecific fragment thereof, which specifically binds to one or more epitopes of a lung tumor-associated polypeptide, variant polypeptide or fragment thereof described herein.

In particular, the present invention includes a method for treating a hyperproliferative disease, e.g., inhibiting tumor formation, tumor growth, tumor invasiveness, and/or metastasis formation in an animal, e.g., a mammal, e.g., a human patient, or prolonging survival of the animal, where the method comprises, consists essentially of, or consists of administering to an animal in need of such treatment an effective amount of a composition comprising, consisting essentially of, or consisting of, in addition to a pharmaceutically acceptable carrier, a binding molecule which specifically binds to a lung tumor-associated polypeptide, variant or fragment thereof. Such lung-tumor associate polypeptides include the following polypeptides and their respective amino acid sequences:

SEQ ID NO:1

RGRRRGGGGRPPPPASSARPPSPAARPLAAPTPAAPACRSPSPGGAPASF
PGRAPRSLASQPAARAAAAPAMPSAKQRGSKGGHGAASPSEKGAHPSGGA
DDVAKKPPPAPQQPPPPPAPHPQQHPQQHPQNQAHGKGGHRGGGGGGGKS
SSSSSASAAAAAAAASSSASCSRRLGRALNFLFYLALVAAAAFSGWCVHH
VLEEVQQVRRSHQDFSRQREELGQGLQGVEQKVQSLQATFGTFESILRSS
QHKQDLTEKAVKQGESEVSRISEVLQKLQNEILKDLSDGIHVVKDARERD
FTSLENTVEERLTELTKSINDNIAIFTEVQKRSQKEINDMKAKVASLEES
EGNKQDLKALKEAVKEIQTSAKSREWDMEALRSTLQTMESDIYTEVRELV
SLKQEQQAEKEAADTERLALQALTEKLLRSEESVSRLPEEIRRLEEELRQ
LKSDSHGPKEDGGFRHSEAFEALQQKSQGLDSRLQHVEDGVLSMQVASAR
QTESLESLLSKSQEHEQRLAALQGRLEGLGSSEADQDGLASTVRSLGETQ
LVLYGDVEELKRSVGELPSTVESLQKVQEQVHTLLSQDQAQAARLPPQDF
LDRLSSLDNLKASVSQVEADLKMLRTAVDSLVAYSVKIETNENNLESAKG
LLDDLRNDLDRLFVKVEKIHEKV

SEQ ID NO:2

MAANVGSMFQYWKRFDLQQLQRELDATATVLANRQDESEQSRKRLIEQSR
EFKKNTPEDLRKQVAPLLKSFQGEIDALSKRSKEAEAAFLNVYKRLIDVP
DPVPALDLGQQLQLKVQRLHDIETENQKLRETLEEYNKEFAEVKNQEVTI
KALKEKIREYEQTLKNQAETIALEKEQKLQNDFAEKERKLQETQMSTTSK
LEEAEHKVQSLQTALEKTRTELFDLKTKYDEETTAKADEIEMIMTDLERA
NQRAEVAQREAETLREQLSSANHSLQLASQIQKAPDVEQAIEVLTRSSLE
VELAAKEREIAQLVEDVQRLQASLTKLRENSASQISQLEQQLSAKNSTLK
QLEEKLKGQADYEEVKKELNILKSMEFAPSEGAGTQDAAKPLEVLLLEKN
RSLQSENAALRISNSDLSGRCAELQVRITEAVATATEQRELIARLEQDLS
IIQSIQRPDAEGAAEHRLEKIPEPIKEATALFYGPAAPASGALPEGQVDS
LLSIISSQRERFRARNQELEAENRLAQHTLQALQSELDSLRADNIKLFEK
IKFLQSYPGRGSGSDDTELRYSSQYEERLDPFSSFSKRERQRKYLSLSPW
DKATLSMGRLVLSNKMARTIGFFYTLFLHCLVFLVLYKLAWSESMERDCA
TFCAKKFADHLHKFHENDNGAAAGDLWQ

SEQ ID NO:3

MRKQHIRCGTGPPRVWGADWSTLRAVMSASVVSVISRFLEEYLSSTPQRL
KLLDAYLLYILLTGALQFGYCLLVGTFPFNSFLSGFISCVGSFILAVCLR
IQINPQNKADFQGISPERAFADFLFASTILHLVVMNFVG

SEQ ID NO:4

RSDAGPGATVSAAAAAATERARRGATMGAQLSTLGHMVLFPVWFLYSLLM
KLFQRSTPAITLESPDIKYPLRLIDREIISHDTRRFRFALPSPQHILGLP
VGQHIYLSARIDGNLVVRPYTPISSDDDKGFVDLVIKVYFKDTHPKFPAG
GKMSQYLESMQIGDTIEFRGPSGLLVYQGKGKFAIRPDKKSNPIIRTVKS
VGMIAGGTGITPMLQVIRAIMKDPDDHTVCHLLFANQTEKDILLRPELEE
LRNKHSARFKLWYTLDRAPEAWDYGQGFVNEEMIRDHLPPPEEEPLVLMC
GPPPMIQYACLPNLDHVGHPTERCFVE

SEQ ID NO:5

EPASRDRAMWLEILLTSVLGFAIYWFISRDKEETLPLEDGWWGPGTRSAA
REDDSIRPFKVETSDEEIHDLHQRIDKFRFTPPLEDSCFHYGFNSNYLKK
VISYWRNEFDWKKQVEILNRYPHFKTKIEGFNSVATARIFYKLMLRLGFQ
EFYIQGGDWGSLICTNMAQLVPSHVKGLHLNMALVLSNFSTLTLLLGQRF
GRFLGLTERDVELLYPVKEKVFYSLMRESGYMHIQCTKPDTVGSALNDSP
VGLAAYILEKFSTWTNTEFRYLEDGGLERKFSLDDLLTNVMLYWTTGTII
SSQRFYKENLGQGWMTQKHERMKVYVPTGFSAFPFELLHTPEKWVRFKYP
KLISYSYMVRGGHFAAFEEPELLAQDIRKF

SEQ ID NO:6

GGGAAAGFLPGGGSGPPSPLLPPLRRRSRRHPPRPTPGRPRQPAGEAQRE
SRNSRPEPTAPGPGRRAEKMNLQPIFWIGLISSVCCVFAQTDENRCLKAN
AKSCGECIQAGPNCGWCTNSTFLQEGMPTSARCDDLEALKKKGCPPDDIE
NPRGSKDIKKNKNVTNRSKGTAEKLKPEDITQIQPQQLVLRLRSGEPQTF
TLKFKRAEDYPIDLYYLMDLSYSMKDDLENVKSLGTDLMNEMRRITSDFR
IGFGSFVEKTVMPYISTTPAKLRNPCTSEQNCTSPFSYKNVLSLTNKGEV
FNELVGKQRISGNLDSPEGGFDATMQVAVCGSLIGWRNVTRLLVFSTDAG
FHFAGDGKLGGIVLPNDGQCHLENNMYTMSHYYDYPSIAHLVQKLSENNI
QTIFAVTEEFQPVYKELKNLIPKSAVGTLSANSSNVIQLIIDAYNSLSSE
VILENGKLSEGVTISYKSYCKNGVNGTGENGRKCSNISIGDEVQFEISIT
SNKCPKKDSDSFKIRPLGFTEEVEVILQYICECECQSEGIPESPKCHEGN
GTFECGACRCNEGRVGRHCECSTDEVNSEDMDAYCRKENSSEICSNNGEC
VCGQCVCRKRDNTNEIYSGKFCECDNFNCDRSNGLICGGNGVCKCRVCEC
NPNYTGSACDCSLDTSTCEASNGQICNGRGICECGVGKCTDPKFQGQTCE
MCQTCLGVCAEHKECVQCRAFNKGEKKDTCTQECSYFNITKVESRDKLPQ
PVQPDPVSHCKEKDVDDCWFYFTYSVNGNNEVMVHVVENPECPTGPDIIP
IVAGVVAGIVLIGLALLLIWKLLMIIHDRREFAKFEKEKMNAKWDTGENP
IYKSAVTTVVNPKYEGK

SEQ ID NO:7

TSLRKRCCPLAISRPGGRDWNSGESFLFCLRVSLHLAISVLQRAGKALRE
TAYPPPAAGSALQLCAPENCQRSWVLARRDGPIRRSLALCSFRPLTRRRC
GSDGGVGGGRGCRGLGRLGWQLRLVAMEWGSESAAVRRHRVGVERREGAA
AAPPPEREARAQEPLVDGCSGGGRTRKRSPGGSGGASRGAGTGLSEVRAA
LGLALYLIALRTLVQLSLQQLVLRGAAGHRGEFDALQARDYLEHITSIGP
RTTGSPENEILTVHYLLEQIKLIEVQSNSLHKISVDVQRPTGSFSIDFLG
GFTSYYDNITNVVVKLEPRDGAQHAVLANCHFDSVANSPGASDDAVSCSV
MLEVLRVLSTSSEALHHAVIFLFNGAEENVLQASHGFITQHPWASLIRAF
INLEAAGVGGKELVFQTGPENPWLVQAYVSAAKHPFASVVAQEVFQSGII
PSDTDFRIYRDFGNIPGIDLAFIENGYIYHTKYDTADRILTDSIQRAGDN
ILAVLKHLATSDMLAAASKYRHGNMVFFDVLGLFVIAYPSRIGSIINYMV
VMGVVLYLGKKFLQPKHKTGNYKKDFLCGLGITLISWFTSLVTVLIIAVF
ISLIGQSLSWYNHFYVSVCLYGTATVAKIILIHTLAKRFYYMNASAQYLG
EVFFDISLFVHCCFLVTLTYQGLCSAFISAVWVAFPLLTKLCVHKDFKQH
GAQGKFIAFYLLGMFIPYLYALYLIWAVFEMFTPILGRSGSEIPPDVVLA
SILAGCTMILSSYFINFIYLAKSTKKTMLTLTLVCAITFLLVCSGTFFPY
SSNPANPKPKRVFLQHMTRTFHDLEGNAVKRDSGIWINGFDYTGISHITP
HIPEINDSIRAHCEENAPLCGFPWYLPVHFLIRKNWYLPAPEVSPRNPPH
FRLISKEQTPWDSIKLTFEATGPSHMSFYVRAHKGSTLSQWSLGNGTPVT
SKGGDYFVFYSHGLQASAWQFWIEVQVSEEHPEGMVTVAIAAHYLSGEDK
RSPQLDALKEKFPDWTFPSAWVCTYDLFVF

SEQ ID NO:8

DLSAREESGAGARPRRRSADSGAAGAGRGGGGEAAGKEEEGESRSRRASM
GRLASRPLLLALLSLALCRGRVVRVPTATLVRVVGTELVIPCNVSDYDGP
SEQNFDWSFSSLGSSFVELASTWEVGFPAQLYQERLQRGEILLRRTANDA
VELHIKNVQPSDQGHYKCSTPSTDATVQGNYEDTVQVKVLADSLHVGPSA
RPPPSLSLREGEPFELRCTAASASPLHTHLALLWEVHRGPARRSVLALTH
EGRFHPGLGYEQRYHSGDVRLDTVGSDAYRLSVSRALSADQGSYRCIVSE
WIAEQGNWQEIQEKAVEVATVVIQPSVLRAAVPKNVSVAEGKELDLTCNI
TTDRADDVRPEVTWSFSRMPDSTLPGSRVLARLDRDSLVHSSPHVALSHV
DARSYHLLVRDVSKENSGYYYCHVSLWAPGHNRSWHKVAEAVSSPAGVGV
TWLEPDYQVYLNASKVPGFADDPTELACRVVDTKSGEANVRETVSWYYRM
NRRSDNVVTSELLAVMDGDWTLKYGERSKQRAQDGDFIFSKEHTDTFNFR
IQRTTEEDRGNYYCVVSAWTKQRNNSWVKSKDVFSKPVNIFWALEDSVLV
VKARQPKPFFAAGNTFEMTCKVSSKNIKSPRYSVLIMAEKPVGDLSSPNE
TKYIISLDQDSVVKLENWTDASRVDGVVLEKVQEDEFRYRMYQTQVSDAG
LYRCMVTAWSPVRGSLWREAATSLSNPIEIDFQTSGPIFNASVHSDTPSV
IRGDLIKLFCIITVEGAALDPDDMAFDVSWFAVHSFGLDKAPVLLSSLDR
KGIVTTSRRDWKSDLSLERVSVLEFLLQVHGSEDQDFGNYYCSVTPWVKS
PTGSWQKEAEIHSKPVFITVKMDVLNAFKYPLLIGVGLSTVIGLLSCLIG
YCSSHWGCKKEVQETRRERRRLMSMEMD

SEQ ID NO:9

LGGPRGRRLPIDCGRCKGRSLWRLVGVLGSAGGGRGVSECERGTGIPNLR
ASRLWRRGGRAQAAMRDRTHELRQGDDSSDEEDKERVALVVHPGTARLGS
PDEEFFHKVRTIRQTIVKLGNKVQELEKQQVTILATPLPEESMKQELQNL
RDEIKQLGREIRLQLKAIEPQKEEADENYNSVNTRMRKTQHGVLSQQFVE
LINKCNSMQSEYREKNVERIRRQLKITNAGMVSDEELEQMLDSGQSEVFV
SNILKDTQVTRQALNEISARHSEIQQLERSIRELHDIFTFLATEVEMQGE
MINRIEKNILSSADYVERGQEHVKTALENQKKARKKKVLIAICVSITVVL
LAVIIGVTVVG

SEQ ID NO:10

MAPPQVLAFGLLLAAATATFAAAQEECVCENYKLAVNCFVNNNRQCQCTS
VGAQNTVICSKLAAKCLVMKAEMNGSKLGRRAKPEGALQNNDGLYDPDCD
ESGLFKAKQCNGTSTCWCVNTAGVRRTDKDTEITCSERVRTYWIIIELKH
KAREKPYDSKSLRTALQKEITTRYQLDPKFITSILYENNVITIDLVQNSS
QKTQNDVDIADVAYYFEKDVKGESLFHSKKMDLTVNGEQLDLDPGQTLIY
YVDEKAPEFSMQGLKAGVIAVIVVVVIAVVAGIVVLVISRKKRMAKYEKA
EIKIEMGEMHRELNA

SEQ ID NO:11

MSGLSGPPARRGPFPLALLLLFLLGPRLVLAISFHLPINSRKCLREEIHK
DLLVTGAYEISDQSGGAGGLRSHLKITDSAGHILYSKEDATKGKFAFTTE
DYDMFEVCFESKGTGRIPDQLVILDMKHGVEAKNYEEIAKVEKLKPLEVE
LRRLEDLSESIVNDFAYMKKREEEMRDTNESTNTRVLYFSIFSMFCLIGL
ATWQVFYLRRFFKAKKLIE

SEQ ID NO:12

GRAAPNGLRGASLPGSGRRVASGEWRVSGGRPAGAGRPEEALAAGSDPRG
AAARLACSAPTPGGGTMPFDFRRFDIYRKVPKDLTQPTYTGAIISICCCL
FILFLFLSELTGFITTEVVNELYVDDPDKDSGGKIDVSLNISLPNLHCEL
VGLDIQDEMGRHEVGHIDNSMKIPLNNGAGCRFEGQFSINKVPGNEHVST
HSATAQPQNPDMTHVIHKLSFGDTLQVQNIHGAFNALGGADRLTSNPLAS
HDYILKIVPTVYEDKSGKQRYSYQYTVANKEYVAYSHTGRIIPAIWFRYD
LSPITVKYTERRQPLYRFITTICAIIGGTFTVAGILDSCIFTASEAWKKI
QLGKMH

SEQ ID NO:13

ASVSQVEADLK

SEQ ID NO:14

KQVAPLLK

SEQ ID NO:15

ADFQGISPER

SEQ ID NO:16

GPSGLLVYQGK

SEQ ID NO:17

FLGLTERDVELLYPVK

SEQ ID NO:18

LLVFSTDAGFHFAGDGK

SEQ ID NO:19

EARAQEPLVDGCSGGGR

SEQ ID NO:20

VPTATLVR

SEQ ID NO:21

LVGVLGSAGGGR

SEQ ID NO:22

TQNDVDIADVAYYFEK

SEQ ID NO:23

RIEDLSFSIVNDFAYMK

SEQ ID NO:24

YDLSPITV

SEQ ID NO:25

RGTGGGRGQQRKLPAAGTGPAQAAYGGRRVGPRVTAGQLGPARSLRVGSP
QHKMLSSFLSPQNGTWADTFSLLLALAVALYLGYYWACVLQRPRLVAGPQ
FLAFLEPHCSITTETFYPTLWCFEGRLQSIFQVLLQSQPLVLYQSDILQT
PDGGQLLLDWAKQPDSSQDPDPTTQPIVLLLPGITGSSQDTYVLHLVNQA
LRDGYQAVVFNNRGCRGEELRTHRAFCASNTEDLETVVNHIKHRYPQAPL
LAVGISFGGILVLNHLAQARQAAGLVAALTLSAGWDSFETTRSLETPLNS
LLENQPLTAGLCQLVERNRKVIEKVVDIDFVLQARTIRQFDERYTSVAFG
YQDCVTYYKAASPRTKIDAIRIPVLYLSAADDPFSPVCALPIQAAQHSPY
VALLITARGGHIGFLEGLLPWQHWYMSRLLHQYAKAIFQDPEGLPDLRAL
LPSEDRNS

SEQ ID NO:26

MRLLRRWAFAALLLSLLPTPGLGTQGPAGALRWGGLPQLGGPGAPEVTEP
SRLVRESSGGEVRKQQLDTRVRQEPPGGPPVHLAQVSFVIPAIFNSNFTL
DLELNHHLLSSQYVERHFSREGTTQHSTGAGDHCYYQGKLRGNPHSFAAL
STCQGLHGVFSDGNLTYIVEPQEVAGPWGAPQGPLPHLIYRTPLLPDPLG
CREPGCLFAVPAQSAPPNRPRLRRKRQVRRGHPTVHSETKYVELIVINDH
QLFEQMRQSVVLTSNFAKSVVNLADVIYKEQLNTRIVLVAMETWADGDKI
QVQDDLLETLARLMVYRREGLPEPSDATHLFSGRTFQSTSSGAAYVGGIG
SLSHGGGVNEYGNMGAMAVTLAQTLGQNLGMMWNKIIRSSAGDCKCPDIW
LGCIMEDTGFYLPRKFSRCSIDEYNQFLQEGGGSCLFNKPLKLLDPPECG
NGFVEAGEECDCGSVQECSRAGGNCCKKCTLTHDAMCSDGLCCRRCKYEP
RGVSCREAVNECDIAETCTGDSSQCPPNLHKLDGYYCDHEQGRCYGGRCK
TRDRQCQVLWGHAAADRFGYEKLNVEGTERGSCGRKGSGWVQCSKQDVLC
GFLLCVNISGAPRLGDLVGDISSVTFYHQGKELDCRGGHVQLADGSDLSY
VEDGTACGPNMLCLDHRCLPASAFNFSTCPGSGERRICSHHGVGSNEGKC
ICQPDWTGKDCSIHNPLPTSPPTGETERYKGPSGTNIIIGSLAGAVLVAA
IVLGGTGWGFKNIRRGRSGGA

SEQ ID NO:27

LLPSLSPEAGPSPIPPLPRLPAPTGPALPAAGPWHVKTWSAPAGPARGTP
AAPRARMRGQAAAPGPVWILAPLLLLLLLLGRRARAAAGADAGPGPEPCA
TLVQGKFFGYFSAAAVFPANASRCSWTLRNPDPRRYTLYMKVAKAPVPCS
GPGRVRTYQFDSFLESTRTYLGVESFDEVLRLCDPSAPLAFLQASKQFLQ
MRRQQPPQHDGLRPRAGPPGPTDDFSVEYLVVGNRNPSRAACQMLGRWLD
AGLAGSRSSHPCGIMQTPCACLGGEAGGPAAGPLAPRGDVCLRDAVAGGP
ENCLTSLTQDRGGHGATGGWKLWSLWGECTRDCGGGLQTRTRTCLPAPGV
EGGGCEGVLEEGRQCNREACGPAGRTSSRSQSLRSTDARRREELGDELQQ
FGFPAIPQTGDPAAEEWSPWSVGSSTGGEGWQTRTRFCVSSSYSTQCSGP
LREQRLCNNSAVCPVHGAWDEWSPWSLCSSTCGRGFRDRTRTCRPPQFGG
NPCEGPEKQTKFCNIALCPGRAVDGNWNEWSSWSACSASCSQGRQQRTRE
CNGPSYGGAECQGHWVETRDCFLQQCPVDGKWQAWASWGSCSVTCGAGSQ
RRERVCSGPFFGGAACQGPQDEYRQCGTQRCPEPHEICDEDNFGAVIWKE
TPAGEVAAVRCPRNATGLILRRCELDEEGIAYWEPPTYIRCVSIDYRNIQ
MMTREHLAKAQRGLPGEGVSEVIQTLVEISQDGTSYSGDLLSTIDVLRNM
TEIFRRAYYSPTPGDVQNFVQILSNLLAEENRDKWEEAQLAGPNAKELFR
LVEDFVDVIGFRMKDLRDAYQVTDNLVLSIHKLPASGATDISFPMKGWRA
TGDWAKVPEDRVTVSKSVFSTGLTEADEASVFVVGTVLYRNLGSFLALQR
NTTVLNSKVISVTVKPPPRSLRTPLEIEFAHMYNGTTNQTCILWDETDVP
SSSAPPQLGPWSWRGCRTVPLDALRTRCLCDRLSTFAILAQLSADANMEK
ATLPSVTLIVGCGVSSLTLLMLVIIYVSVWRYIRSERSVILINFCLSIIS
SNALILIGQTQTRNKVVCTLVAAFLHFFFLSSFCWVLTEAWQSYMAVTGH
LRNRLIRKRFLCLGWGLPALVVAISVGFTKAKGYSTMNYCWLSLEGGLLY
AFVGPAAAVVLVNMVIGILVFNKLVSKDGITDKKLKERAGASLWSSCVVL
PLLALTWMSAVLAVTDRRSALFQILFAVFDSLEGFVIVMVHCILRREVQD
AVKCRVVDRQEEGNGDSGGSFQNGHAQLMTDFEKDVDLACRSVLNKDIAA
CRTATITGTLKRPSLPEEEKLKLAHAKGPPTNFNSLPANVSKLHLHGSPR
YPGGPLPDFPNHSLTLKRDKAPKSSFVGDGDIFKKLDSELSRAQEKALDT
SYVILPTATATLRPKLPKEEPKYSIHIDQMPQTRLIHLSTAPEASLPARS
PPSRQPPSGGPPEAPPAQPPPPPPPPPPPPQQPLPPPPNLEPAPPSLGDP
GEPAAHPGPSTGPSTKNENVATLSVSSLERRKSRYAELDFEKIMHTRKRH
QDMFQDLNRKLQHAAEKDKEVLGPDSKPEKQQTPNKRPWESLRKAHGTPT
WVKKELEPLQPSPLELRSVEWERSGATIPLVGQDIIDLQTE

SEQ ID NO:28

LHSAVRTADLQPKMIPAVVLLLLLLVEQAAALGEPQLCYILDAILFLYGI
VLTLLYCRLKIQVRKAAITSYEKSDGVYTGLSTRNQETYETLKHEKPPQ

SEQ ID NO:29

LRVVSAGRGEAVTCQGARSLSAAWRTWPRAASGHSLSSGDCREAGPRAMG
CFECCIKCLGGIPYASLIATILLYAGVALFCGCGHEALSGTVNILQTYFE
MARTAGDTLDVFTMIDIFKYVIYGIAAAFFVYGSLHSXXXXXXXXXXXXX
XXXXXXXXXXXXXXLQFIMLTYLFMLAWLGVTAFTSLPVYMYFNLWTICR
NTTLVEGANLCLDLRQFGIVTIGEEKKICTVSENFLRMGESTELNMTFHL
FIVALAGAGAAVIAMVHYLMVLSANWAYVKDACRMQKYEDIKSKEEQELH
DIHSTRSKERLNAYT

SEQ ID NO:30

LLESSSEALKTIDELAFKIDLNSTSHVNITTRNLALSVSSLLPGTNAISN
FSIGLPSNNESYFQMDFESGQVDPLASVILPPNLLENLSPEDSVLVRRAQ
FTFFNKTGLFQDVGPQRKTLVSYVMACSIGNITIQNLKDPVQIKIKHTRT
QEVHHPICAFWDLNKNKSFGGWNTSGCVAHRDSDASETVCLCNIIFTHFG
VLMDLPRSASQLDARNTKVLTFISYIGCGISAIFSAATLLTYVAFEKLRR
DYPSKILMNLSTALLFLNLLFLLDGWITSFNVDGLCIAVAVLLHFFLLAT
FTWMGLEAIHMYIALVKVFNTYIRRYILKFCIIGWGLPALVVSVVLASRN
NNEVYGKESYGKEKGDEFCWIQDPVIFYVTCAGYFGVMFFLNIAMFIVVM
VQICGRNGKRSNRTLREEVLRNLRSVVSLTFLLGMTWGFAFFAWGPLNIP
FMYLFSIFNSLQGLFIFIFHCAMKENVQKQWRQHLCCGRFRLADNSDWSK
TATNIIKKSSDNLGKSLSSSSIGSNSTYLTSKSKSSSTTYFKRNSHTDNV
SYEHSFNKSGSLRQCFHGQVLVKTGPC

SEQ ID NO:31

MNRYSAQKQYWKAKQAKQGNIITEAALLKKLQHAAELEQKQNESENKKLL
GEIVKYSNVIQLLHIKSNKYLTVNKRLPALLEKNAMRVSLDAAGNEGSWF
YIHPFWKLRSEGDNIVVGDKVVLMPVNAGQPLHASNIELLDNPGCKEVNA
VTNCNTSWKITLFMKYSSYREDVLKGGDVVRLFHAEQEKFLTCDEYEKKQ
HIFLRTTLRQSATSATSSKALWEIEVVHHDPCRGGAGQWNSLFRFKIILA
TGNYLAAELNPDYRDAQNEGKNVRDGVPPTSKKKRQAGEKIMYTLVSVPH
GNDIASLFELDATFLQRADCLVPRNSYVRLRHLCTNTWVTSTSIPIDTDE
ERPVMLKIGTCQTKEDKEAFAIVSVPLSEVRDLDFANDANKVLATTVKIC
LENGTITQNERRFVTKILEDLIFFVADVPNNGQEVLDVVITKPNRERQKL
MREQNILAQVFGILKAPFKEKAGEGSMLRLEDLGDQRYAPYKYMLRLGYR
VLRHSQQDYRKNQEYIAKNFCVMQSQIGYDILAEDTITALLHNNRKLLEK
HITAKEIETFVSLLRRNREPRFLDYLSDLCVSNTTAIPVTQELICKFMLS
PGNADILIQTKVVSMQADNPMESSILSDDIDDEEVWLYWIDSNKEPHGKA
IRIILAQEAKEGTKADLEVLTYYRYQLNLFARMCLDRQYLATNQISTQLS
VDLILRCVSDESLPFDLRASFCRLMLHMHVDRDPQESVVPVRYARLWTEI
PTKITIHEYDSITDSSRNDMKRKFALTMEFVEEYLKEVVNQPFPFGDKEK
NKLTFEVVHLARNLIYFGFYSFSELLRLTRTLLAILDIVQAPMSSYFERL
SKFQDGGNNVMRTIHGVGEMMTQMVLSRGSIFPMSVPDVPPSIHPSKQGS
PTEHEDVTVMDTKLKIIEILQFILSVRLDYRISYMLSIYKKEFGEDNDNA
ETSASGSPDTLLPSAIVPDIDEIAAQAETMFAGRKEKNPVQLDDEGGRTF
LRVLIHLIMHDYPPLLSGALQLLFKHFSQRAEVLQAFKQVQLLVSNQDVD
NYKQIKADLDQLRLTVEKSELWVEKSSNYENGEIGESQVKGGEEPIEESN
ILSPVQDGTKKLPQIDSNKSNNYRIVKEILIRLSKLCVQNKKCRNQHQRL
LKNMGAHSVVLDLLQIPYEKNDEKMNEVMNLAHTFLQNFCRGNPQNQVLL
HKHLNLFLTPGLLEAETMRHIFMNNYHLCNEISERVVQHFVHCIETHGRI
IVEYLRFLQTIVKADGKYVKKCQDMVMTELINGGEDVLIFYNDRASFPIL
LHMMCSERDRGDESGPLAYHITLVELLAACTEGKNVYTEIKCNSLLPLDD
IVRVVTHDDCIPEVKIAYVNFVNHCYVDTEVEMKEIYTSNHIWKLFENFL
VDMARVCNTTTDRKHADIFLEKCVTESIMNIVSGFFNSPFSDNSTSLQTH
QPVFIQLLQSAFRIYNCTWPNIPAQKASVESCIRTLAEVAKNRGIAIIPV
DLDSQVNTLFMKSHSNMVQRAAMGWRLSARSGPRFKEALGGPAWDYRNII
EKLQDVVASLEHQFSPMMQAEFSVLVDVLYSPELLFPEGSDARIRCGAEM
SKLINHTKKLMEKEEKLCIKILQTLREMLEKKDSFVEEGNTLRIKILLNR
YFKGDYSIGVNGHLSGAYSKTAQVGGSFSGQDSDKMGISMSDIQCLLDKE
GASELVIDVIVNTKNDRIFSEGIFLGIALLEGGNTQTQYSFYQQLHEQKK
SEKFFKVLYDRMKAAQKEIRSTVTVNTIDLGNKKRDDDNIELMTSGPRMR
VRDSTLHLKEGMKGQLTEASSATSKAYCVYRREMDPEIDIMCTGPEAGNT
EEKSAEEVTMSPAIAIMQPILRFLQLLCENHNRELQNFLRNQNNKTNYNL
VCETLQFLDCICGSTFITGGLGLLGLYINEKNVALVNQNLESLTEYCQGP
CHENQTCIATHESNGIDIIIALILNDINPLGKYRMDLVLQLKNNASKLLL
AIMESRHDSENAERILFNMRPRELVDVMLKNAYNQGLECDHGDDEGGDDG
VSPKDVGHNIYILAHQLARHNKLLQQMLKPGSDPDEGDEALKYYANHTAQ
IEIVRHDRTMEQIVFPVPNICEYLTRESKCRVFNTTERDEQGSKVNDFFQ
QTEDLYNEMKWQKLKIRNNPALFWFSRIIISLWGSISFNLAVFINLAVAL
FYPFGDDGDEGTLSPLFSVLLWIAVAICTSMLFFFSKPVGIRPFLVSIML
RSIYTIGLGPTLILLGAANLCNKIVFLVSFVGNRGTFTRGYRAVILDMAF
LYHVAYVLVCMLGLFVHEFFYSFLLFDLVYREETLLNVIKSVTRNGRSII
LTAVLALILVYLFSIIGFLFLKDDFTMEVDRLKNRTPVTGSHQVPTMTLT
TMMEACAKENCSPTIPASNTADEEYEDGIERTCDTLLMCIVTVLNQGLRN
GGGVGDVLRRPSKDEPLFAARVVYDLLFYFIVIIIVLNLIFGVHDTFADL
RSEKQKKEEILKTTCFICGLERDKFDNKTVSFEEHIKSEHNMWHYLYFIV
LVKVKDPTEYTGPESYVAQMIVEKNLDWFPRMRAMSLVSNEGDSEQNEIR
SLQEKLESTMSLVKQLSGQLAELKEQMTEQRKNKQRLGFLGSNTPHVNTI
HMPPH

SEQ ID NO:32

MVKKLVMAQKRGETRALCLGVTMVVCAVITYYILVTTVLPLYQKSVWTQE
SKCHLIETNIRDQEELKGKKVPQYPCLWVNVSAAGRWAVLYHTEDTRDQN
QQVLNWRDGDTSLYPCQVCEPVPNCPCPRG

SEQ ID NO:33

MVVALRYVWPLLLCSPCLLIQIPEEYEGHHVMEPPVITEQSPRRLVVFPT
DDISLKCEASGKPEVQFRWTRDGVHFKPKEELGVTVYQSPHSGSFTITGN
NSNFAQRFQGIYRCFASNKLGTAMSHEIRLMAEGAPKWPKETVKPVEVEE
GESVVLPGNPPPSAEPLRIYWMNSKILHIKQDERVTMGQNGNLYFANVLT
SDNHSDYICHAHFPGTRTIIQKEPIDLRVKATNSMIDRKPRLLFPTNSSS
HLVALQGQPLVLECIAEGFPTPTIKWLRPSGPMPADRVTYQNHNKTLQLL
KVGEEDDGEYRCLAENSLGSARHAYYVTVEAAPYWLHKPQSHLYGPGETA
RLDCQVQGRPQPEVTWRINGIPVEELAKDQKYRIQRGALILSNVQPSDTM
VTQCEARNRHGLLLANAYIYVVQLPAKILTADNQTYMAVQGSTAYLLCKA
FGAPVPSVQWLDEDGTFFVLQDERFFPYANGTLGIRDLQANDTGRYECLA
ANDQNNVTIMANLKVKDATQITQGPRSTIEKKGSRVTFTCQASFDPSLQP
SITWRGDGRDLQELGDSDKYFIEDGRLVIHSLDYSDQGNYSCVASTELDV
VESRAQLLVVGSPGPVPRLVLSDLHLLTQSQVRVSWSPAEDHNAPIEKYD
IEFEDKEMAPEKWYSLGKVPGNQTSTTLKLSPYVHYTFRVTAINKYGPGE
PSPVSETVVTPEAAPEKNPVDVKGEGNETFITNMVITWKPLRWMDWNAPQ
VQYRVQWRPQGTRGPWQEQIVSDPFLVVSNTSTFVPYEIKVQAVNSQGKG
PEPQVTIGYSGEDYPQAILPELEGIEILNSSAVLVKWRPVDLAQVKGHLR
GYNVTYWREGSQRKHSKRHIHKDHVVVPANTTSVILSGLRPYSSYHLEVQ
AFNGRGSGPASEFTFSTPEGVPGHPEALHLECQSNTSLLLRWQPPLSHNG
VLTGYVLSYHPLDEGGKGQLSFNLRDPELRTHNLTDLSPHLRYFQLQATI
TKEGPGEAIVREGGTMALSGLSDFGNISATAGENYSVVSWVPKEGQCNFR
FHILFKALGEEKGGASLSPQYVSYNQSSYTQWDLQPDTDYEIHLFKERMF
RHQMAVKTNGTGRVRLPPAGFATEGWFIGFVSAIILLLLVLLILCFIKRS
KGGKYSVKDKEDTQVDSEARPMKDETFGEYRSLESDNEEKAFGSSQPSLN
GDIKPLGSDDSLADYGGSVDVQFNEDGSFIGQYSGKEKEAAGGNDSSGAT
SPINPAVALE

SEQ ID NO:34

MKGNHSRKTAAFVRACVAYCFITIPSLAGIFTRLNLYLHSGQVALANQCL
SQADAFFKAAISLVPEVPKMINIDGKMRPSESFLLEFLGNFFSTLLIVPD
HPEHGVLFLVRELLNVIQDYTWEDNSDEKIRIYTCVLHLLSAMSQETYLY
HIDKVDSNDSLYGGDSKFLAENNKLCETVMAQILEHLKTLAKDEALKRQS
SLGLSFFNSILAIIGDLRNNKLNQLSVNLWHLAQRHGCADTRTMVRSLE

SEQ ID NO:35

RDSCCAEEPCGTRGCARARALWPRRGDSEAHWGLPARGRPRRPARGLRLC
APSPEEDACRHRARAAGLNACLPGAAAALPSAGVGSGTRRAPGGRRAQAG
YTLPESAEFAASAGGPAGPDGRGVCGPRRVLRSGPGTGGTLSAGAAAAER
TWGGGHAPVRSLEPSGAPRGPARVGGRSGPHSPRARSSQRAPDKMARPVR
GGLGAPRRSPGLLLLWLLLLRLEPVTAAAGPRAPCAAAGTCAGDSLDCGG
RGLAALPGDLPSWTRSLNLSYNKLSEIDPAGFEDLPNLQEVYLNNNELTA
VPSLGAASSHVVSLFLQHNKIRSVEGSQLKAYLSLEVLDLSLNITEVNTC
FPHGPPRELNLAGNRIGTLELGAFDGLSRSLLTLRISKNRITQLPVRAFK
LPRLTQLDLNRNRIRLIEGLTFQGLNSLEVLKLQRNMSKLTDGAFWGLSK
MHVLHLEYNSLVEVNSGSLYGLTALHQLHLSNSIARIHRKGWSFCQKLHE
LVLSFNNLTRLDEESLAELSSLSVLRLSHNSISHIAEGAFKGLRSLRVLD
LDHNEISGTIEDTSGAFSGLDSLSKLTLFGNKIKSVAKRAFSGLEGLEHL
NLGGNAIRSVQFDAFVKMKNLKELHISSDSFLCDCQLKWLPPWLIGRMLQ
MVTATCAHPESLKGQSIFSVPPESFVCDDFLKPQIITQPETTMAMVGKDI
RFTCSAASSSSSPMTFAWKKDNEVLTNADMENFVHVHAQDGEVMEYTTIL
HLRQVTFGHEGRYQCVITNHFGSTYSHKARLTVNVLPSFTKTPHDITIRT
TTMARLECAATGHPNPQIAWQKDGGTDFPAARERRMHVMPDDDVFFITDV
KIDDAGVYSCTAQNSAGSISANATLTVLETPSLVVPLEDRVVSVGETVAL
QCKATGNPPPRITWFKGDRPLSLTERHHLTPDNQLLVVQNVVAEDAGRYT
CEMSNTLGTERAHSQLSVLPAAGCRKDGTTVGIFTSSIVLTSLVWVCIIY
QTRKKSEEYSVTNTDETVVPPDVPSYLSSQGTLSDRQETVVRTEGGPQAN
GHIESNGVCPRDASHFPEPDTHSVAGRQPKLCAGSAYHKEPWKAMEKAEG
TPGPHKMEHGGRVVCSDCNTEVDCYSRGQAFHPQPVSRDSAQPSAPNGPE
PGGSDQEHSPHHQCSRTAAGSCPECQGSLYPSNHDRMLTAVKKKPMASLD
GKGDSSWTLARLYHPDSTELQPASSLTSGSPERAEAQYLLVSNGHLPKAC
DASPESTPLTGQLPGKQRVPLLLAPKS

SEQ ID NO:36

LGPRVAGVAVAVSPGSLSPLCVWIHVGAGFQSLVLRRSRAGASPSQNPAL
PPERFPGEEGTTSFLKARPRDLMTFEDVAVEFSQWEWGQLNPAQKDLYRE
VMLENFRNLAILGLLVSKPYVICQLEEGGEPFMVEREISTGAHSDWKKRS
KSKESMPSWGISKEELFQVVSVEKHIQDVLQFSKLKAACGCDGQLEMQQI
KQERLHLKQMSTIHKSATTLSRDYKWNGFGRSLGLRSVLVNQHSILMGEG
SYKCDTEFRQTLGGNNSQRTHPEKKSGKCNECGKSFHFQSELRRHQRCHT
GEKPYECSDCGRMGHISSLIKHQRTHTGEKIPYECSECGRAFSQSSSLVL
HYRFHTGEKPYKCNECGRAFGHTSSLIKHQRTHTGEKPYECRECGRTFSQ
SSSLIVHYRFHTGEKPYKCNKCGRAFSQSSSLTQHYEHTGEKPYKCNECG
RAFAHTASLIKHQRSHAGKKTL

SEQ ID NO:37

LVRASRLRGRAHVCSSHCSCWAVELPQGARGTFAAAMKGARWRRVIPWVS
LSCLCLCLLPHVVPGTTEDTLITGSKTAAPVTSTGSTTATLEGQSTAASS
RTSNQDISASSQNHQTKSTETTSKAQTDTLTQMMTSTLFSSPSVHNVMET
APPDEMTTSFPSSVTNTLMMTSKTITMTTSTDSTLGNTEETSTAGTESST
PVTSAVSITAGQEGQSRTTSWRTSIQDTSASSQNHWTRSTQTTRESQTST
LTHRTTSTPSFSPSVHNVTGTVSQKTSPSGETATSSLCSVTNTSMMTSEK
ITVTFSTGSTLGNPGETSSVPVTGSLMPVTSAALVTFDPEGQSPATFSRT
STQDSKNHQTQSVETTRVSQINTLNTLTPVTTSTVLSSPSGFNPSGTVSQ
ETFPSGETTTSSPSSVSNTFLVTSKVFRMPTSRDSTLGNTEETSLSVSGT
ISAITSKVSTIWWSDTLSTALSPSSLPPKISTAFHTQQSEGAETTGRPHE
RSSFSPGVSQEIFTLHETTTWPSSFSSKGHTTWSQTELPSTSTGAATRLV
TGNPSTGTAGTWRVPSKVSAIGEPGEPTTYSSHSTTLPKTTGAGAQTQWT
QETGTTGEALLSSPSYSVTQMIKTATSPSSSPMLDRHTSQQITTAPSTNH
STIHSTSTSPQESPAVSQRGHTQAPQTTQESQTTRSVSPMTDTKTVTTPG
SSFTASGHSPSEIVPQDAPTISAATTFAPAPTGDGWETQAIYFFAIQAAP
SSHDATLGPSGGTSLSKTGALTLANSVVSTPGGPEGQWTSASASTSPDTA
AAMTHTHQAESTEASGQTQTSEPASSGSRTTSAGTATPSSSGASGTLPSG
SEGISTSGETTRFSSNPSRDSHTTQSTLTELLSASASHGAIPVSTGMASS
IVPGTFHPTLSEASTAGRIPTGQSSPTSPSASPQETAAISRMAQTQRTRT
SRGSDTISLASQATDTFSTVPPTPPSITSTGLTSPQTETHTLSPSGSGKT
FTTALISNATPLPVTSTSSASTGHAPTLAVSSATSASTVSSDSPLKMETP
GMTTPSLKTDGGRRTATSPPPTTSQTIISTIPSTAMHTRSTAPILPERGV
SLFPYGAGAGDLEFVRRTVDFTSPLFKPATGFPLGSSLRDSLYFTDNGQI
IFPESDYQIFSYPNPLPTGFTGRDPVALVAPFWDDADFSTGRGTTFYQEY
ETFYGEHSLLVQQAESWIRKMTNNGGYKARWALKVTWVNAHAYPAWWTLG
SNTYQAILSTDGSRSYALFLYQSGGMQWDVAQRSGNPVLMGFSSGDGYFE
NSPLMSQPVWERYRPDRFLNSNSGLQGLQFYRLHREERPNYRLECLQWLK
SQPRWPSWGWNQVSCPCSWQQGRRDLRFQPVSIGRWGLGSRQLCSFTSWR
GGVCCSYGPWGEFREGWHVQRPWQLAQELEPQSWCCRWNDTYLCALYQQR
RPHVGCATTYPPQPAWMFGDPHITTLDGVSYTFNGLGDFLLVGAQDGNSS
FLLQGRTAQTGSAQATNFIAFAAQYRSSSLGPVTVQWLLEPHDAIRVLLD
NQTVTFQPDHEDGGGQETFNATGVLLSRNGSEVSASFDGWATVSVIALSM
LHASASLPPEYQNRTEGLLGVWNNPEDDFRMPNGSTIPPGSPEEMLFHFG
MTWQINGTGLLGKRNDQLPSNFTPVFYSQLQKNSSWAEHLISNCDGDSSC
IYDTLALRNASIGLHTREVSKNYEQANATLNQYPPSINGGRVIEAYKGQT
TLIQYTSNAEDANFTLRDSCTDLELFENGTLLWTPKSLEPFTLEILARSA
KIGLASALQPRTVVCHCNAESQCLYNQTSRVGNSSLEVAGCKCDGGTFGR
YCEGSEDACEEPCFPSVHCVPGKGCEACPPNLTGDGRHCAALGSSFLCQN
QSCPVNYCYNQGHCYISQTLGCQPMCTCPPAFTDSRCFLAGNNFSPTVNL
ELPLRVIQLLLSEEENASMAEVNASVAYRLGTLDMRAFLRNSQVERIDSA
APASGSPIQHWMVISEFQYRPRGPVIDFLNNQLLAAVVEAFLYHVPRRSE
EPRNDVVFQPISGEDVRDVTALNVSTLKAYIFRCDGYKGYDLVYSPQSGF
TCVSPCSRGYCDHGGQCQHLPSGPRCSCVSFSIYTAWGEHCEHLSMKLDA
FFGIFFGALGGLLLLGVGTFVVLRFWGCSGARFSYFLNSAEALP

SEQ ID NO:38

AAAGLLGALHLVMTLVVAAARAEKEGGCPPAASLRRGCHPALAEAGRAGP
GGRAAAGAPAQSWAVGYRPEPGPRGARRTEWPSLSVIPSRAFPRLLSLPF
QNFLTSRTFLPLGPLGRRGIFFGFIAANRYILGHFCFLGCGWLHADRAYF
KMW

SEQ ID NO:39

MVYIHGGSYMEGTGNMIDGSILASYGNVIVITINYRLGILGFLSTGDQAA
KGNYGLLDQIQALRWIEENVGAFGGDPKRVWGSGAGASCVSLLTLSHYSE
GLFQKAIIQSGTALSSWAVNYQPAKYTRILADKVGCNMLDTTDMVECLKN
KNYKELIQQTITPATYHGPVIDGDVIPDDPQILMEQGEFLNYDIMLGVNQ
GEGLKFVDGIVDNEDGVTPNDFDFSVSNFVDNLYGYPEGKDTLRETIKFM
YTDWADKENPETRRKTLVALFTDHQWVAPAVATADLHAQYGSPTYFYAFY
HHCQSEMKPSWADSAHGDEVPYVFGIPMIGPTELFSGNFSKNDVMLSAVV
MTYWTNFAKTGDPNQPVPQDTKFIHTKPNRFEEVAWSKYNPKDQLYLHIG
LKPRVRDHYRATKVAFWLELVPHLHNLNEWQYVSTTTKVPPPDMTSFPYG
TRRSPAKIWPYKRPAITPANNPKHSKDPKKTGPEDTTVLIETKRDYSTEL
SVTIAVGASLLFLNILAFAALYYKKDKRRHETHRHPSPQRNYFNDITHIQ
NEELMSLQMKQLEHDHECESLQAHDTLRLTCPPDYTLTLRRSPDDWFMTP
NTITMWNTLMGMQPLHTFKTFSGGQNSTNLPHGHSTTRV

SEQ ID NO:40

MDMFPLTWWLALYFSRHQVRGQPDPPCGGRLNSKDAGYITSPGYPQDYPS
HQNCEWIEPNQKIVLNFNPHFEIEKHDCKYDFIEIRDGDSESADLLGKHC
GNIAPPTIISSGSMLYIRFTSDYARQGAGFSLRYEIFKTGSEDCSKNFTS
PNGTIESPGFPEKYPHNLDCTFTILAKPKMEIILQFLWDLEHDPLQVGEG
DCKYDWLDIWDGIPHVGPLIGKYCGTKTPSELRSSTGILSLTFHTDMAVA
KDGFSARYYLVHQEPLENFQCNVPLGMESGRIANEQISASSTYSDGRWTP
QQSRLHGDDNGWTPNLDSNKEYLQVDLRFLTMLTAIATQGAISRETQNGY
YVKSYKLEVSTNGEDWMVYRHGKNHKVFQANNDATEVVLNKLHAPLLTRF
VRPQTWHSGIALRLELFGCRVTDAPCSNMLGMLSGLIADSQISASSTQEY
LWSPSARLVSSRSGWFPRIPQAQPGEEWLQVDLGTPKTVKGVIIQGARGG
DSITAVEARAFVRKVSYSLNGKDWEYIQDPRTQQPKLFEGNMHYDTPDIR
RFDPIPAQYVRVYPERWSPAGIGMRLEVLGGDWTDSKPTVETLGPTVKSE
EYPYPTEEEATECGENCSFEDDKDLQLPSGFNCNDFLEEPCGWMYDHAKW
LRTTWASSSSPNDRTFPDDRFLRLQSDSQREGQYARLISPPVHLPRSPVC
MEFQYQATGGRGVALQVVREASQESKLLWVIREDQGGEWKHGRIIILSYD
MEYQIVFEGVIGKGRSGEIAIDDIRISTDVPLENCMEPISAFAGENFKVD
IPEIHEREGYEDEIDDEYEVDWSNSSSATSGSGAPSTDKEKSWLYTLDPI
LITILAMSSLGVLLGATCAGLLLYCTCSYSGLSSRSCTTLENYNFELYDG
LKHKVKMNHQKCCSEA

SEQ ID NO:41

PQASLPALLSEPAAGEGRRRKRLREAGIWASARLAPRPGPWHCEPAREPR
SARGAPGPLPPHAPAALKPERGPGGRAGPGPAVGMASGSRWRPTPPPLLL
LLLLALAAGLEFGGGPGQWARYARWAGAASSGELSFSLRTNATRALLLYL
DDGGDCDFLELLLVDGRLRLRFTLSCAEPATLQLDTPVADDRWHMVLLTR
DARRTALAVDGEARAEVRSKRREMQVASDLFVGGIPPDVLSALTLSTVKY
EPPFRGLLANLKLGERPPALLGSQGLRGATADPLCAPARNPCANGGLCTV
LAPGEVGCDGSHTGFGGKFCSEEEHPMEGPAHLTLNSEVGSLLFSEGGAG
RGGAGDVHQPTKGKEEFVATFKGNEFFCYDLSHNPIQSSTDEITLAFRTL
QRNGLMLHTGKSADYVNLSLKSGAVWLVINLGSGAFEALVEPVNGKFNDN
AWHDVRVTRNLRQHAGIGHAMVNKLHYLVTISVDGILTTTGYTQEDYTML
GSDDFFYIGGSPNTADLPGSPVSNNFMGCLKDVVYKNNDFKLELSRLAKE
GDPKMKLQGDLSFRCEDVAALDPVTFESPEAFVALPRWSAKRTGSISLDF
TTTEPNGLLLFSQGRRAGGGAGSHSSAQRADYFAMELLDGHLYLLLDMGS
GGIKLRASSRKVNDGEWCHVDFQRDGRKGSISVNSRSTPFLATGDSEILD
LESELYLGGLPEGGRVDLPLPPEVWTAALRAGYVGCVDLFIDGRSRDLRG
LAEAQGAVGVAPFCSRETLKQCASAPCRNGGVCREGWNRFICDCIGTGFL
GRVCEREATVLSYDGSMYMKIMLPNAMHTEAEDVSLRFMSQRAYGLMMAT
TSRESADTLRLELDGGQMKLTVNLDCLRVGCAPSKGPETLFAGHKLNDNE
WHTVRVVRRGKSLQLSVDNVTVEGQMAGAHMRLEFHMETGIMTERRFISV
VPSNFIGHLSGLVFNGQPYMDQCKDGDITYCELNARFGLRAIVADPVTFK
SRSSYLALATLQAYASMHLFFQFKTTAPDGLLLFNSGNGNDFIVIELVKG
YIHYVFDLGNGPSLMKGNSDKPVNDNQWHNVVVSRDPGNVHTLKIDSRTV
TQHSNGARNLDLKGELYIGGLSKNMFSNLPKLVASRDGFQGCLASVDLNG
RLPDLIADALHRIGQVERGGDGPSTCTEESCANQGVCLQQWDGFTCDCTM
TSYGGPVCNDPGTTYIFGKGGALITYTWPPNDRPSTRMDRLAVGFSTTQR
SAVLVRVDSASGLGDYLQLHIDQGTVGVIFNVGTDDITIDEPNAIVSDGK
YHVVRFTRSGGNATLQVDSWPVNERYPAGNFDNERLAIARQRIPYRLGRV
VDEWLLDKGRQLTIFNSQAAIKIGGRDQGRPFQGQVSGLYYNGLKVLALA
AESDPNVRTEGHLRLVGEGPSVLLSAETTATTLLADMATTIMETTTTMAT
TTTRRGRSPTLRDSTTQNTDDLLVASAECPSDDEDLEECEPSTGGELILP
IITEDSLDPPPVATRSPFVPPPPTFYPFLTGVGATQDTLPPPAAPPSGGP
CQAERDDSDCEEPIEASGFASGEVFDSSLPPTDDEDFYTTFPLVTDRTTL
LSPRKPAPRPNLRTDGATGAPGVLFAPSAPAPNLPAGKMNHRDPLQPLLE
NPPLGPGAPTSFEPRRPPPLRPGVTSAPGFPHLPTANPTGPGERGPPGAV
EVIRESSSTTGMVVGIVAAAALCILILLYAMYKYNRDEGSYQVDQSRNYI
SNSAQSNGAVVKEKAPAAPKTPSKAKKNKDKEYYV

SEQ ID NO:42

MMCRNFIRNISPFFPLFFKYFSMYDKQYKFCSYVFLFQCLYAKLSVSYNF
INKFHCKMDHTGDRGNISTSSKPASTSGKSELSSKHSRSLKPDGRMSRTI
TADQKKPRGTESLSASESLILKSDAAKLRSDSHSRSLSPNHNTLQTLKSD
GRMPSSSRAESPGPGSRLSSPKPKTLPANRSSPSGASSPRSSSPHDKNLP
QKSTAPVKTKIDPPRERSKSDSYTLDPDTLRKKKMPLTEPLRGRSTSPKP
KSSTDSPGSENRAPSPHVVQENLHSEVVEVCTSSTLKTNSLTDSTCDDSS
EFKSVDEGSNKVHFSIGKAPLKDEQEMRASPKISRKCANRHTRPKKEKSS
FLFKGDGSKPLEPAKQAMSPSVAECAASFLWHEGIVHDAMACSSFLKFHP
ELSKEHAPIRSSLNSQQPTEEKETKLKNRHSLEISSALNMFNIAPHGPDI
SKMGSINKNKVLSMLKEPPLHEKCEDGKTETTFEMSMHNTMKSKSPLPLT
LQHLVAFWEDISLATIKAASQNMIFPSPGSCAVLKKKECEKENKKSKKEK
KKKEKAEVRPRGNLFGEMAQLAVGGPEKDTICELCGESHPYPVTYHMRQA
HPGCGRYAGGQGYNSIGHFCGGWAGNCGDGGIGGSTWYLVGDRCREKYLV
CDRCREKYLREKQAAAREKVKQSRRKPMQVKTPRALPTMEAHQASS

SEQ ID NO:43

MSEHVEPAAPGPGPNGGGGGPAPARGPRTPNLNPNPLIVRDRLFHALFFK
MAVTYSRLFPPAFRRLFEFFVLLKALFVLFVLAYIHIVFSRSPTNCLEHV
RDKWPREGILRVEVRHNSSRAPVFLQFCDSGGRGSFPGLAVEPGSNLDME
DEEEEELTMEMFGNSSIKVPGRPQFELDIEPKVFKPPSSTEALNDSQEFP
FPETPTKVWPQDEYIVEYSLEYGFLRLSQATRQRLSIPVMVVTLDPTRDQ
CFGDRFSRLLLDEFLGYDDILMSSVKGLAENEENKGFLRNVVSGEHYRFV
SMWMARTSYLAAFMMVIFTLSVSMLLRYSHHQIFVFIVDLLQMLEMNMAI
AFPAAPLLTVILALVGMEAIMSEFFNDTTTAFYIILIVWLADQYDAICCH
TSTSKRHWLRFFYLYHFAFYAYHYRFNGQYSSLALVTSWLFIQHSMIYFF
HHYELPAILQQVRIQEMLLQAPPLGPGTPTALPDDMNNSGAPATAPDSAG
QPPALGPVSPGASGSPGPVAAAPSSLVAAAASVAAAAGGDLGWMAETAAI
ITDASFLSGLSASLLERRPASPLGPAGGLPPQDSVPPSDSAASDTTPLGA
AVGGPSPASMAPTEAPSEVGS

SEQ ID NO:44

RPRTPPRAAAATARTPPPLPATAEPSMGVAGRNRPGAAWAVLLLLLLLPP
LLLLAGAVPPGRGRAAGPQEDVDECAQGLDDCHADALCQNTPTSYKCSCK
PGYQGEGRQGEDIDECGNELNGGCVHDCLNIPGNYRCTCFDGFMLAHDGH
NCLDVDECLENNGGCQHTCVNVMGSYECCCKEGFFLSDNQHTCIHRSEEG
LSCMNKDHGGSHICKEAPRGSVACECRLPGFELAKNQRDCILTCNHGNGG
CQHSCDDTADGPECSCHPQYKMHTDGRSCLEREDTVLEVTESNTTSVVDG
DKRVKRRLLMETCAVNNGGCDRTCKDTSTGVHCSCPVGFTLQLDGKTCKD
IDECQTRNGGCDHFCKNIVGSFDCGCKKGFKLLTDEKSCQDVDECSLDRT
CDHSCINHPGTFACACNRGYTLYGFTHCGDTNECSINNGGCQQVCNTVGS
YECQCHPGYKLHWNKKDCVEVKGLLPTSVSPRVSLHCGKSGGGDGCFLRC
HSGIHLSSDVTTIRTSVTFKLNEGKCSLKNAELFPEGLRPALPEKHSSVK
ESFRYVNLTCSSGKQVPGAPGRPSTPKEMFITVEFELETNQKEVTASCDL
SCIVKRTEKLRKAIRTLRKAVHREQFHLQLSGMNLDVAKKPRTSERQAES
CGVGQGHAENQCVSCRAGTYYDGARERCILCPNGTFQNEEGQMTCEPCPR
PGNSGALKTPEAWNMSECGGLCQPGEYSADGFAPCHLGALGTFQPEAGRT
SCFPCGGGLATKHQGATSFQDCETRVQCSPGHFYNTTTHRCIRCPVGTYQ
PEFGKNNCVSCPGNTTTDFDGSTNITQCKNRRCGGELGDFTGYIESPNYP
GNYPANTECTWTPPPKRMLIVVPEIFLPIEDDCGDYLVMRKTSSSNSVTT
YETCQTYERPIAFTSRSKKLWIQFKSNEGNSARGFQVPYVTYDEDYQELI
EDIVRDGRLYASENHQEILKDKKLIKALFDVLAHPQNYFKYTAQESREMF
PRSFIRLLRSKVSRFLRPYK

SEQ ID NO:45

LHFLWFCFKSHFLLGKLLPNTRTLLLFEHSDIVVISLLSVLFTSSGGGPA
KTRGAAFFIIAVIGLLLFDNDDLMAKMAEHPEGHHDSALTHMLYTAIAFL
GVADHKGGVLLLVLALCCKVGFHTASRKLSVDVGGAKRLQALSHLVSVLL
LCPWVIVLSVTTESKVESWFSLIMPFATVIFFVMILDFYSICSVKMEVSK
CARYGSFPIFISALLFGNFWTHPITDQLRAMNKAAHQESTEHVLSGGVVV
SAIFFILSANILSSPSKRGQKGTLIGYSPEGTPLYNFMGDAFQHSSQSIP
RFIKESLKQILEESDSRQIFYFLCLNLLFTFVELFYGVLTNSLGLISDGF
HMLFDCSALVMGLFAALMSRWKATRIFSYGYGRIEILSGFNGLFLIVIAF
FVFMESVARLIDPPELDTHMLTPVSVGGLIVNLIGICAFSHAHSHAHGAS
QGSCHSSDHSHSHHMHGHSDHGHGHSHGSAGGGMNANMRGVFLHVLADTL
GSIGVIVSTVLIEQFGWFIADPLCSLFIAILIFLSVVPLIKDACQVLLLR
LPPEYEKELHIALEKVLYVISSLLSSLKITFLKSLLEVKQTTK

SEQ ID NO:46

MEPGDAARPGSGRATGAPPPRLLLLPLLLGWGLRVAAAASASSSGAAAED
SSAMEELATEKEAEESHRQDSVSLLTFILLLTLTILTIWLFKHRRVRFLH
ETGLAMIYGLIVGVILRYGTPATSGRDKSLSCTQEDRAFSTLLVNVSGKF
FEYTLKGEISPGKINSVEQNDMLRKVTFDPEILLPPIIFHAGYSLKKRHF
FRNLGSILAYAFLGTAVSCFIIGNLMYGVVKLMKIMGQLSDKFYYTDCLF
FGAIISATDPVTVLAIFNELHADVDLYALLFGESVLNDAVAIVLSSSIVA
YQPAGLNTHAFDAAAFFKSVGWLGIFSGSFTMGAVTGVNANVTKFTKLHC
FPLLETALFFLMSWSTFLLAEACGFTGVVAVLFCGITQAHYTYNNLSVES
RSRTKQLFEVLHFLAENFIFSYMGLALFTFQKHVFSPIFIIGAFVAIFLG
RAAHIYPLSFFLNLGRRHKIGWNFQHMMMFSGLRGAMAFALAIRDTASYA
RQMMFTTTLLIVFFTVWIIGGGTTPMLSWLNIRVGVEEPSEEDQNEHHWQ
YFRVGVDPDQDPPPNNDSFQVLQGDGPDSARGNRTKQESAWIFRLWYSFD
HNYLKPILTHSGPPLTTLPAWCGLLARCLTSPQDNQEPLREEDSDFILTE
GDLTLTYGDSTVTANGSSSSHTASTSLEGSRRTKSSSEEVLERDLGMGDQ
KVSSRGTRLVFPLEDNA

SEQ ID NO:47

MERPWGAADGLSRWPHGLGLLLLLQLLPPSTLSQDRLDAPPPPAAPLPRW
SGPIGVSWGLRAAAAGGAFPRGGRWRRSPGEDEECGRVRDFVAKLANNTH
QHWDDLRGSVSLSWVGDSTGVILVLTTFHVPLVIMTFGQSKLYRSEDYGK
INFKDITDLINNTFIRTEFGMAIGPENSGKVVLTAEVSGGSRGGRWRSSD
FANFVQTDLPFHPLTQMMYSPQNSDYLLALSTENGLWVSKNFGGKWEEIH
KAVCLAKWGSDNTIFFTTYANGSCKADLGALELWRTSDLGKSFKTIGVKI
YSFGLGGRFLFASVMADKDTTRRIHVSTDQGDTWSMAQLPSVGQEQFYSI
LAANDDMVFMHVDEPGDTGFGTIFTSDDRGIVYSKSLDRHLYTTTGGETD
FTNVTSLRGVYITSVLSEDNSIQTMITFDQGGRWTHLENSECDATAKNKN
ECSLHIHASYSISQKLNVPMAPLSEPNAVGIVIAHGSVGDAISVMVPDVY
ISDDGGYSWTKMLEGPHYYTILDSGGIIVAIEHSSRPNVIKFSTDEGQCW
QTYTFTRDPIYFTGLASEPGARSMNISIWGFTESFLTSQWVSYTIDFKDI
LERNCEEKDYTIWLAHSTDPEDYEDGCILGYKEQFLRLRKSSVCQNGRDY
VVTKQPSICLCSLEDFLCDFGYYRPENDSKCVEQPELKGHDLEFCLYGRE
EHLTTNGYRKIPGDKCQGGVPVREVKDLKKKCTSNFLSPEKQNSKSNSVP
IILAWGLMLVTVVAGVLIVKKYVCGGRFLVHRYSVLQQHAEANGVDGVDA
LDTASHTNKSGYHDDSDEDLLE

SEQ ID NO:48

LDLFNFFHCISVLLQGKVMITLTELKCLADAQSSYHILKPWWDVFWYYIT
LIMLLVAVAGALQLTQSRVLCCLPCKVEFDNHCAVPWDILKASMNTSSNI
PGTPLPLPLRIQNDLHRQQYSYIDAVCYEKQLHWFAKFFPYLVLLHTLIF
AACSNFWLHYPSTSSRLEHFVAILHKCFDSPWTTRALSETVAEQSVRPLK
LSKSKILLSSSGCSADIDSGKQSLPYPQPGLESAGIESPTSSVLDKKEGE
QAKAIFEKVKRFRMHVEQKDIIYRVYLKQIIVKVILFVLIITYVPYFLTH
ITLEIDCSVDVQAFTGYKRYQCVYSLAEIFKVLASFYVILVILYGLTSSY
SLWWMLRSSLKQYSFEALREKSNYSDIPDVKNDFAFILHLADQYDPLYSK
RFSIFLSEVSENKLKQINLNNEWTVEKLKSKIVKNAQDKIELHLFMLNGL
PDNVFELTEMEVLSLELIPEVKLPSAVSQLVNLKELRVYHSSLVVDHPAL
AFLEENLKILRLKFTEMGKIPRWVFHLKNLKELYLSGCVLPEQLSTMQLE
GFQDLKNLRTLYLKSSLSRIPQVVTDLLPSLQKLSLDNEGSKLVVLNNLK
KMVNLKSLELISCDLERIPHSIFSLNNLHELDLLKTVEEIISFQHLQNLS
CLKLWHNNIAYIPAQIGALSNLEQLSLDHNIENLPLQLFLCTKLHYLDLS
YLTFIPEEIQYLSNLQYFAVVTNNNIEMLPDGLFQCKKLQGLLLGKNSLM
NLSPHVGELSNLTHLELIGNYLETLPPELEGCQSLKRNCLIVEENLLNTL
PLPVTERLQTCLDKC

SEQ ID NO:49

SHPLAQNGFEYTNCCGAARGREDTSASETARSDGDSEPRIHRATHRSSED
DARMMSASRLAGTLIPAMAFLSCVPESWEPCVEVPNITYQCMELPDNLPF
STKNLDLSFNPLRHLGSYSFFSFPELQVLDLSRCEIQTIEDGAYQSLSHL
STLILTGNPIQSLALGAFSGLSSLQKLVAVETNLASLENFPIGHLKTLKE
LNVAHNLIQSFKLPEYFSNLTNLEHLDLSSNKIQSIYCTDLRVLHQMPLL
NLSLDLSLDLSLNPMNFIQPGAFKEIRLHKLTLRNNFDSLNVMKTCIQGL
AGLEVHRLVLGEFRNEGNLEKFDKSALEGLCNLTIEEFRLAYLDYYLDDI
IDLFNCLTNVSSFSLVSVTIERVKDFSYNFGWQHLELVNCKFGQFPTLKL
KSLKRLTFTSNKGGNAFSEVDLPSLEFLDLSRNGLSFKGCCSQSDFGTTS
LKYLDLSFNGVITTMSSNFLGLEQLEHLDFQHSNLKQMSEFSVFLSLRNL
IYLDISHTHTRVAFNGIFNGLSSLEVLKMAGNSFQENFLPDIFTELRNLT
FLDLSQCQLEQLSPTSLSSLQVLNMSHNNFFSLDTFPYKCLNSLQVLDYS
LNHIMTSKKQELQHFPSSLAFLNLTQNDFACTCEHQSFLQWIKDQRQLLV
EVERMECATPSDKQGMPVLSLNITCQMNKTIIGVSVLSVLVVSVVAVLVY
KFYFHLMLLAGCIKYGRGENIYDAFVIYSSQDEDWVRNELVKNLEEGVPP
FQLCLHYRDFIPGVAIAANIIHEGFHKSRKVIVVVSQHFIQSRWCIFEYE
IAQTWQFLSSRAGIIFIVLQKVEKTLLRQQVELYRLLSRNTYLEWEDSVL
GRHIFWRRLRKALLDGKSWNPEGTVGTGCNWQEATSI

SEQ ID NO:50

MQAARVDYIAPWWVVWLHSVPHVGLRLQPVNSTFSPGDESYQESLLFLGL
VAAVCLGLNLIFLVAYLVCACHGRRDDAVQTKQHHSGCITWTAVVAGLIC
CAAVGVGFYGNSETNDGAYQLMYSLDDANHTFSGIDALVSGTTQKMKVDL
EQHLARLSEIFAARGDYLQTLKFIQQMAGSVVVQLSGLPVWREVTMELTK
LSDQTGYVEYYRWLSYLLLFILDLVICLIACLGLAKRSKCLLASMLCCGA
LSLLLSWASLAADGSAAVATSDFCVAPDTFILNVTEGQISTEVTRYYLYC
SQSGSSPFQQTLTTFQRALTTMQIQVAGLLQFAVPLFSTAEEDLLAIQLL
LNSSESSLHQLTCRGLHKDYLDALAGICYDGLQGLLYLGLFSFLAALAFS
TMICAGPRAWKHFTTRNRDYDDIDDDDPFNPQAWRMAAHSPPRGQLHSFC
SYSSGLGSQTSLQPPAQTISNAPVSEYMNQAMLFGRNPRYEPLIGRASPP
PTYSPSMRATYLSVADEHLRHYGNQFPA

SEQ ID NO:51

LRVGEAVSCVTAFLYGIVILILEALLNFWKRDLLGIMVRLFKDSSLRECI
C

SEQ ID NO:52

MAAGTAARKAAPVLEAPPQQEQLSHTKLSAEDTWNLQQERMYKMHRGHDS
MHVEMILIFLCVLVIAQIVLVQWRQRHGRSYNLVTLLQMWVVPLYFTIKL
YWWRFLSMWGMFSVITSYILFRATRKPLSGRTPRLVYKKWFLLIYKLSYA
FGVVGYLAIMFTMCGFNLFFSMDFGWSLFYGLYYGVMGRDFAEICSDYMA
STIGFYSVSRLPTRSLSDNICAVCGQKIIVELDEEGLIENTYQLSCNHVF
HEFCIRGWCIVGKKQTCPYCKEKVDLKRMISNPWERTHFLYGQILDWLRY
LVAWQPVVIGIVQGIIYSLGLE

Variant polypeptides of the present invention include polypeptides which are at least 70%, 75%, 80%, 85%, 90%, 95% or 100% identical to lung tumor-associated polypeptides selected from the group consisting of SEQ ID NOs: 1 to 52.

In the above embodiments, exemplary “fragments” of a lung tumor associate-polypeptide or variant polypeptide include but are not limited to: a fragment comprising, consisting essentially of, or consisting of a fragment selected from the group consisting of: SEQ ID NOs:13-24.

In the above embodiments, exemplary “fragments” of a lung tumor associate-polypeptide or variant polypeptide include but are not limited to: a fragment comprising, consisting essentially of, or consisting of a fragment selected from the group consisting of: amino acids 58-80 of SEQ ID NO:25; amino acids 137-369 of SEQ ID NO:25; amino acids 158-277 of SEQ ID NO:25; amino acids 173-299 of SEQ ID NO:25; amino acids 176-422 of SEQ ID NO:25; amino acids 205-283 of SEQ ID NO:25; amino acids 402-424 of SEQ ID NO:25; amino acids 100-216 of SEQ ID NO:26; amino acids 239-438 of SEQ ID NO:26; amino acids 453-529 of SEQ ID NO:26; amino acids 677-709 of SEQ ID NO:26; amino acids 65-88 of SEQ ID NO:27; amino acids 289-297 of SEQ ID NO:27; amino acids 321-370 of SEQ ID NO:27; amino acids 414-462 of SEQ ID NO:27; amino acids 418-429 of SEQ ID NO:27; amino acids 469-517 of SEQ ID NO:27; amino acids 473-486 of SEQ ID NO:27; amino acids 527-575 of SEQ ID NO:27; amino acids 533-549 of SEQ ID NO:27; amino acids 582-630 of SEQ ID NO:27; amino acids 634-695 of SEQ ID NO:27; amino acids 936-994 of SEQ ID NO:27; amino acids 982-1248 of SEQ ID NO:27; amino acids 993-1339 of SEQ ID NO:27. amino acids 1000-1247 of SEQ ID NO:27; amino acids 1005-1249 of SEQ ID NO:27; amino acids 1021-1243 of SEQ ID NO:27; amino acids 1023-1240 of SEQ ID NO:27; amino acids 1302-1541 of SEQ ID NO:27; amino acids 75-95 of SEQ ID NO:28; amino acids 50-291 of SEQ ID NO:29; amino acids 154-207 of SEQ ID NO:30; amino acids 211-469 of SEQ ID NO:30; amino acids 216-476 of SEQ ID NO:30; amino acids 229-419 of SEQ ID NO:30; amino acids 235-475 of SEQ ID NO:30; amino acids 253-455 of SEQ ID NO:30; amino acids 286-304 of SEQ ID NO:30; amino acids 291-482 of SEQ ID NO:30; amino acids 331-353 of SEQ ID NO:30; amino acids 485-496 of SEQ ID NO:30; amino acids 50-104 of SEQ ID NO:31; amino acids 111-161 of SEQ ID NO:31; amino acids 169-225 of SEQ ID NO:31; amino acids 232-340 of SEQ ID NO:31; amino acids 409-615 of SEQ ID NO:31; amino acids 1119-1293 of SEQ ID NO:31; amino acids 2252-2478 of SEQ ID NO:31; amino acids 2-122 of SEQ ID NO:32; amino acids 34-133 of SEQ ID NO:33; amino acids 35-133 of SEQ ID NO:33; amino acids 50-116 of SEQ ID NO:33; amino acids 138-172 of SEQ ID NO:33;amino acids 144-159 of SEQ ID NO:33; amino acids 151-211 of SEQ ID NO:33; amino acids 235-400 of SEQ ID NO:33; amino acids 242-330 of SEQ ID NO:33; amino acids 249-320 of SEQ ID NO:33; amino acids 257-314 of SEQ ID NO:33; amino acids 293-307 of SEQ ID NO:33; amino acids 333-422 of SEQ ID NO:33; amino acids 347-406 of SEQ ID NO:33; amino acids 426-515 of SEQ ID NO:33; amino acids 441-499 of SEQ ID NO:33; amino acids 471-511 of SEQ ID NO:33; amino acids 517-609 of SEQ ID NO:33; amino acids 518-609 of SEQ ID NO:33; amino acids 532-593 of SEQ ID NO:33; amino acids 612-701 of SEQ ID NO:33; amino acids 714-800 of SEQ ID NO:33; amino acids 743-763 of SEQ ID NO:33; amino acids 812-907 of SEQ ID NO:33; amino acids 918-1005 of SEQ ID NO:33; amino acids 1017-1097 of SEQ ID NO:33; amino acids 1130-1148 of SEQ ID NO:33; amino acids 34-67 of SEQ ID NO:34; amino acids 234-261 of SEQ ID NO:35; amino acids 263-286 of SEQ ID NO:35; amino acids 287-308 of SEQ ID NO:35; amino acids 310-330 of SEQ ID NO:35; amino acids 334-357 of SEQ ID NO:35; amino acids 358-381 of SEQ ID NO:35; amino acids 383-405 of SEQ ID NO:35; amino acids 406-429 of SEQ ID NO:35; amino acids 430-453 of SEQ ID NO:35; amino acids 454-477 of SEQ ID NO:35; amino acids 478-501 of SEQ ID NO:35; amino acids 502-528 of SEQ ID NO:35; amino acids 502-525 of SEQ ID NO:35; amino acids 526-549 of SEQ ID NO:35; amino acids 539-552 of SEQ ID NO:35; amino acids 550-573 of SEQ ID NO:35; amino acids 550-563 of SEQ ID NO:35; amino acids 577-600 of SEQ ID NO:35; amino acids 601-624 of SEQ ID NO:35; amino acids 625-645 of SEQ ID NO:35; amino acids 625-654 of SEQ ID NO:35; amino acids 659-684 of SEQ ID NO:35; amino acids 689-790 of SEQ ID NO:35; amino acids 703-773 of SEQ ID NO:35; amino acids 793-884 of SEQ ID NO:35; amino acids 807-868 of SEQ ID NO:35; amino acids 867-883 of SEQ ID NO:35; amino acids 887-975 of SEQ ID NO:35; amino acids 897-974 of SEQ ID NO:35; amino acids 901-959 of SEQ ID NO:35; amino acids 74-114 of SEQ ID NO:36; amino acids 274-286 of SEQ ID NO:36; amino acids 277-299 of SEQ ID NO:36; amino acids 277-294 of SEQ ID NO:36; amino acids 277-287 of SEQ ID NO:36; amino acids 302-314 of SEQ ID NO:36; amino acids 305-327 of SEQ ID NO:36; amino acids 305-315 of SEQ ID NO:36; amino acids 307-348 of SEQ ID NO:36; amino acids 330-342 of SEQ ID NO:36; amino acids 332-340 of SEQ ID NO:36 amino acids 333-355 of SEQ ID NO:36; amino acids 333-343 of SEQ ID NO:36; amino acids 335-356 of SEQ ID NO:36; amino acids 358-370 of SEQ ID NO:36; amino acids 360-368 of SEQ ID NO:36; amino acids 361-383 of SEQ ID NO:36; amino acids 361-371 of SEQ ID NO:36; amino acids 386-398 of SEQ ID NO:36; amino acids 389-411 of SEQ ID NO:36; amino acids 389-399 of SEQ ID NO:36; amino acids 390-412 of SEQ ID NO:36; amino acids 391-412 of SEQ ID NO:36; amino acids 402-440 of SEQ ID NO:36; amino acids 414-426 of SEQ ID NO:36; amino acids 416-424 of SEQ ID NO:36; amino acids 417-439 of SEQ ID NO:36; amino acids 417-427 of SEQ ID NO:36; amino acids 417-440 of SEQ ID NO:36; amino acids 442-455 of SEQ ID NO:36; amino acids 444-452 of SEQ ID NO:36; amino acids 445-467of SEQ ID NO:36;amino acids 445-455 of SEQ ID NO:36; amino acids 719-727 of SEQ ID NO:37; amino acids 1255-1340 of SEQ ID NO:37; amino acids 1341-1456 of SEQ ID NO:37; amino acids 1362-1389 of SEQ ID NO:37; amino acids 1470-1642 of SEQ ID NO:37; amino acids 1476-1487 of SEQ ID NO:37; amino acids 1644-1662 of SEQ ID NO:37; amino acids 1675-1684 of SEQ ID NO:37; amino acids 1824-1859 of SEQ ID NO:37; amino acids 1885-1896 of SEQ ID NO:37; amino acids 1910-1944 of SEQ ID NO:37; amino acids 1928-1943 of SEQ ID NO:37; amino acids 2112-2147 of SEQ ID NO:37; amino acids 2121-2161 of SEQ ID NO:37; amino acids 1422 of SEQ ID NO:39; amino acids 77-87 of SEQ ID NO:39; amino acids 309-322 of SEQ ID NO:39; amino acids 507-537 of SEQ ID NO:39; amino acids 519-538 of SEQ ID NO:39; amino acids 591-602 of SEQ ID NO:39; amino acids 1-16 of SEQ ID NO:40; amino acids 28-139 of SEQ ID NO:40; amino acids 121-141 of SEQ ID NO:40; amino acids 149-264 of SEQ ID NO:40; amino acids 292-424 of SEQ ID NO:40; amino acids 449-589 of SEQ ID NO:40; amino acids 646-802 of SEQ ID NO:40; amino acids 141-274 of SEQ ID NO:41; amino acids 141-271 of SEQ ID NO:41; amino acids 290-325 of SEQ ID NO:41; amino acids 402-546 of SEQ ID NO:41; amino acids 402-543 of SEQ ID NO:41; amino acids 605-753 of SEQ ID NO:41; amino acids 605-750 of SEQ ID NO:41; amino acids 778-800 of SEQ ID NO:41; amino acids 844-974 of SEQ ID NO:41; amino acids 890-925 of SEQ ID NO:41; amino acids 1030-1161 of SEQ ID NO:41; amino acids 1030-1158 of SEQ ID NO:41; amino acids 1184-1216 of SEQ ID NO:41; amino acids 1253-1402 of SEQ ID NO:41; amino acids 129-157 of SEQ ID NO:42; amino acids 647-655 of SEQ ID NO:42; amino acids 39-49 of SEQ ID NO:43; amino acids 74-91 of SEQ ID NO:43; amino acids 264-281 of SEQ ID NO:43; amino acids 356-372 of SEQ ID NO:43; amino acids 71-110 of SEQ ID NO:44; amino acids 75-110 of SEQ ID NO:44; amino acids 158-193 of SEQ ID NO:44; amino acids 203-239 of SEQ ID NO:44; amino acids 312-347 of SEQ ID NO:44; amino acids 349-388 of SEQ ID NO:44; amino acids 390-427 of SEQ ID NO:44; amino acids 429-468 of SEQ ID NO:44; amino acids 433-468 of SEQ ID NO:44; amino acids 670-720 of SEQ ID NO:44; amino acids 727-774 of SEQ ID NO:44; amino acids 783-830 of SEQ ID NO:44; amino acids 835-944 of SEQ ID NO:44; amino acids 4-516 of SEQ ID NO:45; amino acids 21-199 of SEQ ID NO:45; amino acids 37-418 of SEQ ID NO:45; amino acids 101-115 of SEQ ID NO:45; amino acids 110-268 of SEQ ID NO:45; amino acids 320-534 of SEQ ID NO:45; amino acids 321-596 of SEQ ID NO:45; amino acids 353-591 of SEQ ID NO:45; amino acids 438-456 of SEQ ID NO:45; amino acids 26-50 of SEQ ID NO:46; amino acids 35-457 of SEQ ID NO: 46; amino acids 62-468 of SEQ ID NO: 46; amino acids 74-534 of SEQ ID NO: 46; amino acids 324-496 of SEQ ID NO: 46; amino acids 364-378 of SEQ ID NO: 46; amino acids 441-470 of SEQ ID NO: 46; amino acids 552-565 of SEQ ID NO: 46; amino acids 145-156 of SEQ ID NO:47; amino acids 240-251 of SEQ ID NO:47; amino acids 287-298 of SEQ ID NO:47; amino acids 328-339 of SEQ ID NO:47; amino acids 377-388 of SEQ ID NO:47; 428-439 of SEQ ID NO:47; 506-517 of SEQ ID NO:47; amino acids 548-559 of SEQ ID NO:47; amino acids 506-525 of SEQ ID NO:48; amino acids 529-556 of SEQ ID NO:48; amino acids 604-626 of SEQ ID NO:48; amino acids 627-651 of SEQ ID NO:48;amino acids 652-674 of SEQ ID NO:48; amino acids 675-697 of SEQ ID NO:48; amino acids 698-720 of SEQ ID NO:48; amino acids 721-743 of SEQ ID NO:48; amino acids 744-766 of SEQ ID NO:48; amino acids 767-789 of SEQ ID NO:48; amino acids 51-95 of SEQ ID NO:49; amino acids 110-133 of SEQ ID NO:49; amino acids 134-157 of SEQ ID NO:49; amino acids 158-181 of SEQ ID NO:49; amino acids 182-205 of SEQ ID NO:49; amino acids 206-230 of SEQ ID NO:49; amino acids 231-254 of SEQ ID NO:49; amino acids 244-261 of SEQ ID NO:49; amino acids 371-381 of SEQ ID NO:49; amino acids 388-409 of SEQ ID NO:49; amino acids 429-446 of SEQ ID NO:49; amino acids 455-477 of SEQ ID NO:49; amino acids 478-499 of SEQ ID NO:49; amino acids 503-526 of SEQ ID NO:49; amino acids 527-551 of SEQ ID NO:49; amino acids 541-556 of SEQ ID NO:49; amino acids 552-575 of SEQ ID NO:49; amino acids 576-599 of SEQ ID NO:49; amino acids 576-602 of SEQ ID NO:49; amino acids 600-630 of SEQ ID NO:49; amino acids 661-683 of SEQ ID NO:49; amino acids 684-704 of SEQ ID NO:49; amino acids 731-869 of SEQ ID NO:49; amino acids 27-433 of SEQ ID NO:50; amino acids 104-154 of SEQ ID NO:52; amino acids 174-186 of SEQ ID NO:52; amino acids 226-279 of SEQ ID NO:52; amino acids 227-276 of SEQ ID NO:52; amino acids 227-223 of SEQ ID NO:52; or amino acids 273-282 of SEQ ID NO:52.

Additionally, exemplary fragments of the lung tumor-associated polypeptides and variant polypeptides include but are not limited to fragments of the extracellular domains of the lung tumor-associated polypeptides described herein. For example, fragments selected from the group consisting of: amino acids 90-458 of SEQ ID NO:25; amino acids 1-735 of SEQ ID NO:26; amino acids 1-1003 of SEQ ID NO:27; amino acids 1061-1064 of SEQ ID NO:27; amino acids 1129-1147 of SEQ ID NO:27;. amino acids 1214-1640 of SEQ ID NO:27; amino acids 33-36 of SEQ ID NO:28; amino acids 81-112 of SEQ ID NO:29; 171-173 of SEQ ID NO:29; 271-315 of SEQ ID NO:29; amino acids 1-219 of SEQ ID NO:30; amino acids 278-282 of SEQ ID NO:30; amino acids 348-380 of SEQ ID NO:30; amino acids 447-449 of SEQ ID NO:30; amino acids 1-2163 of SEQ ID NO:31; amino acids 2221-2239 of SEQ ID NO:31; amino acids 2306-2332 of SEQ ID NO:31; amino acids 2483-2639.of SEQ ID NO:31; amino acids 40-130 of SEQ ID NO:32; amino acids 1-1120 of SEQ ID NO:33; amino acids 35-248 of SEQ ID NO:34; amino acids 1-986 of SEQ ID NO:35; amino acids 28-473 of SEQ ID NO:36; amino acids 1-2159 of SEQ ID NO:37; amino acids 1-119 of SEQ ID NO:38; amino acids 1-506 of SEQ ID NO:39; amino acids 1-867 of SEQ ID NO:40; amino acids 1-1719 of SEQ ID NO:41; amino acids 53-693 of SEQ ID NO:42; amino acids 1-66 of SEQ ID NO:43; amino acids 331-349 of SEQ ID NO:43; amino acids 394-407 of SEQ ID NO:43; amino acids 456-626 of SEQ ID NO:43; amino acids 59-1025 of SEQ ID NO:44; amino acids 1-19 of SEQ ID NO:45; amino acids 69-95 of SEQ ID NO:45; amino acids 159-167 of SEQ ID NO:45; amino acids 226-244 of SEQ ID NO:45; amino acids 342-350 of SEQ ID NO:45; amino acids 409-422 of SEQ ID NO:45; amino acids 516-519 of SEQ ID NO:45; amino acids 1-71 of SEQ ID NO:46; amino acids 121-181 of SEQ NO:46; amino acids 235-248 of SEQ ID NO:46; amino acids 306-324 of SEQ ID NO:46; amino acids 395-413 of SEQ ID NO:46; amino acids 461-474 of SEQ ID NO:46; amino acids 533-725 of SEQ ID NO:46; amino acids 1-755 of SEQ ID NO:47; amino acids 1-43 of SEQ ID NO:48; amino acids 161-279 of SEQ ID NO:48; amino acids 346-821 of SEQ ID NO:48; amino acids 1-688 of SEQ ID NO:49; amino acids 31-44 of SEQ ID NO:50; amino acids 110-212 of SEQ ID NO:50; amino acids 263-387 of SEQ ID NO:50; amino acids 27-54 of SEQ ID NO:51; amino acids 1-50 of SEQ ID NO:52; amino acids 99-101 of SEQ ID NO:52; amino acids 163-176 of SEQ ID NO:52; and amino acids 326-328 of SEQ ID NO:52.

As known in the art, “sequence identity” between two polypeptides is determined by comparing the amino acid sequence of one polypeptide to the sequence of a second polypeptide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for UNIX, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule which specifically binds to at least one epitope of a lung tumor-associate peptide described herein, where the epitope comprises, consists essentially of, or consists of at least about four to five amino acids amino acids of a polypeptide selected from the group consisting of SEQ ID NOs:1 to 52, at least seven, at least nine, or between at least about 15 to about 30 amino acids of a polypeptide selected from the group consisting of SEQ ID NOs:1 to 52. The amino acids of a given epitope of a polypeptide selected from the group consisting of SEQ ID NOs:1 to 52 as described may be, but need not be contiguous. In certain embodiments, the at least one epitope of a lung tumor-associated polypeptide comprises, consists essentially of, or consists of a non-linear epitope formed by the extracellular domain of a lung associated polypeptide as expressed on the surface of a cell. Thus, in certain embodiments the at least one epitope of a lung tumor-associated polypeptide comprises, consists essentially of, or consists of at least 4, at least 5, at least 6, at least 7, at least 8, at least 9, at least 10, at least 11, at least 12, at least 13, at least 14, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of a polypeptide selected from the group consisting of SEQ ID NOs:1-52, where non-contiguous amino acids form an epitope through protein folding.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule which specifically binds to at least one epitope of a lung tumor-associated polypeptide, where the epitope comprises, consists essentially of, or consists of, in addition to one, two, three, four, five, six or more contiguous or non-contiguous amino acids of a polypeptide selected from the group consisting of SEQ ID NOs:1 to 52 as described above, an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

More specifically, the present invention provides a method of treating cancer in a human, comprising administering to a human in need of treatment a composition comprising an effective amount of a lung tumor-associate polypeptide-specific antibody or immunospecific fragment thereof, and a pharmaceutically acceptable carrier. Types of cancer to be treated include, but are not limited to, colon cancer, lung cancer, breast cancer, pancreatic cancer, and prostate cancer.

A binding molecule for use in the present invention is typically a binding polypeptide, in particular an antibody or immunospecific fragment thereof. In certain embodiments, an antibody or fragment thereof binds specifically to at least one epitope of a lung tumor-associated polypeptide or fragment or variant described above, i.e., binds to such an epitope more readily than it would bind to an unrelated, or random epitope; binds preferentially to at least one epitope of a lung tumor-associated polypeptide or fragment or variant described above, i.e., binds to such an epitope more readily than it would bind to a related, similar, homologous, or analogous epitope; competitively inhibits binding of a reference antibody which itself binds specifically or preferentially to a certain epitope of a lung tumor-associated polypeptide or fragment or variant described above; or binds to at least one epitope of a lung tumor-associated polypeptide or fragment or variant described above with an affinity characterized by a dissociation constant K_{D} of less than about 5×10⁻²M, about 10⁻²M, about 5×10⁻³M, about 10⁻³M, about 5×10⁻⁴M, about 10⁻⁴M, about 5×10⁻⁵M, about 10⁻⁵M, about 5×10⁻⁶M, about 10⁻⁶M, about 5×10⁻⁷M, about 10⁻⁷M, about 5×10⁻⁸M, about 10⁻⁸M, about 5×10⁻⁹M, about 10⁻⁹M, about 5×10⁻¹⁰M, about 10⁻¹⁰M, about 5×10⁻¹¹M, about 10⁻¹¹M, about 5×10⁻¹²M, about 10⁻¹²M, about 5×10⁻¹³M, about 10⁻¹³M, about 5×10⁻¹⁴M, about 10⁻¹⁴M, about 5×10⁻¹⁵M, or about 10⁻¹⁵M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻²M” might include, for example, from 0.05 M to 0.005 M.

In specific embodiments, binding molecules, e.g., antibodies or immunospecific fragments thereof for use in the diagnostic and treatment methods disclosed herein bind lung tumor-associated polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. More preferably, binding molecules, e.g., antibodies or immunospecific fragments thereof for use in the diagnostic and treatment methods disclosed herein bind lung tumor-associated polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻⁶, 10⁻⁶ sec⁻¹, 5×10⁻⁷ sec⁻¹ or 10⁻⁷ sec⁻¹ .

In other embodiments, binding molecules, e.g., antibodies or immunospecific fragments thereof for use in the diagnostic and treatment methods disclosed herein bind lung tumor-associated polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, binding molecules, e.g., antibodies or immunospecific fragments thereof for use in the diagnostic and treatment methods disclosed herein bind lung tumor-associated polypeptides or fragments or variants thereof with an on rate (k(on)) greater than or equal to 10⁵M⁻¹ sec⁻¹, 5×10⁵M⁻¹ sec⁻¹, 10⁶M⁻¹ sec⁻¹, or 5×106 M⁻¹ sec⁻¹ or 10⁷ M⁻¹ sec⁻¹.

In various embodiments, one or more binding molecules as described above is an antagonist of an activity attributable to a lung tumor-associated polypeptide described herein. For example binding of the binding molecule to a lung-tumor associated polypeptide expressed in tumor-associated vascular tissue, blocks angiogenesis in the tissue thereby inhibiting tumor growth or spread. Alternatively, binding of the binding molecule to a lung tumor-associated polypeptide, as expressed in the cellular membrane of a tumor cell may inhibit the ability of the lung tumor-associated polypeptide to facilitate invasiveness of the tumor cell, e.g., through prevention or retardation of metastatic growth, or through prevention or retardation of tumor spread to adjacent tissues. In addition, binding of the binding molecule to a lung tumor-associated polypeptide, as expressed in the cellular membrane of tumor cell may facilitate killing of the tumor cell, for example through effector functions such as complement-mediated lysis or antibody-dependent cellular cytotoxicity.

### DIAGNOSTIC OR PROGNOSTIC METHODS USING LUNG TUMOR-ASSOCIATED POLYPEPTIDE-SPECIFIC BINDING MOLECULES

Lung tumor-associated polypeptide-specific binding molecules, e.g., antibodies, or fragments, derivatives, or analogs thereof, can be used for diagnostic purposes to detect, diagnose, or monitor diseases, disorders, and/or conditions associated with the aberrant expression and/or activity of the lung tumor-associated polypeptides described herein.

Lung tumor-associated polypeptide-specific binding molecules disclosed herein, e.g., antibodies or fragments thereof, are useful for diagnosis, treatment, prevention and/or prognosis of hyperproliferative disorders in mammals, preferably humans. Such disorders include, but are not limited to, cancer, neoplasms, tumors and/or as described under elsewhere herein, especially lung tumor-associated polypeptide-associated cancers such as lung cancers, bronchogenic cancers, small cell lung cancers, non-small cell lung cancers, oat cell carcinomas, small cell undifferentiated carcinomas, sqamous cell carcinomas, adenocarcinomas, large-cell undifferentiated carcinomas, pancreatic cancers, cervical cancers, ovarian cancers, liver canncers, bladder cancers, breast cancers, colon cancers, renal cancers, prostate cancers, testicular cancers, thyroid cancers, head and neck cancers, carcinoid tumors, adenoid cystic carcinomas, and hamartomas. In a preferred embodiment, such lung tumor-associated polypeptide-associated cancers include, but are not limited to, lung cancers, non-small cell lung cancers, sqamous cell carcinomas, adenocarcinomas, pancreatic cancers, cervical cancers, renal cancers, prostate cancers, and testicular cancers.

In particular, it is believed that certain tumor-associated tissues express significantly enhanced levels of the polypeptides, disclosed herein, when compared to corresponding “standard” levels. Indeed, the proteins described herein were identified based on their increased expression in malignant lung cells relative to nonmalignant lung cells.

For example, binding molecules, e.g., antibodies (and antibody fragments) directed against lung tumor-associated polypeptides, variants and fragments thereof may be used to detect particular tissues expressing these proteins. These diagnostic assays may be performed in vivo or in vitro, such as, for example, on biopsy tissue or autopsy tissue.

Thus, the invention provides a diagnostic method useful during diagnosis of a cancers and other hyperproliferative disorders, which involves measuring the expression level of lung tumor-associated polypeptides described herein in tissue and blood or other bodily fluids, which may contain secreted forms of lung tumor-associated polypeptides, variant polypeptides, or fragments thereof, from an individual and comparing the measured expression level with a standard lung tumor-associated polypeptide expression levels in normal tissue, whereby an increase in the expression level compared to the standard is indicative of a disorder.

With respect to cancer, the presence of a relatively high amount of lung tumor-associated polypeptides in biopsied tissue from an individual may indicate the presence of a tumor or other malignant growth, may indicate a predisposition for the development of such malignancies or tumors, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Lung tumor-associated polypeptide-specific binding molecules can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin. Suitable assays are described in more detail elsewhere herein.

One aspect of the invention is a method for the in vivo detection or diagnosis of a hyperproliferative disease or disorder associated with aberrant expression of a lung tumor-associated polypeptide, variant or fragment thereof in an animal, preferably a mammal and most preferably a human. In one embodiment, diagnosis comprises: a) administering (for example, parenterally, subcutaneously, or intraperitoneally) to a subject an effective amount of a labeled binding molecule, e.g., an antibody or fragment thereof, which specifically binds to a lung tumor-associated polypeptide described herein; b) waiting for a time interval following the administering for permitting the labeled binding molecule to preferentially concentrate at sites in the subject where the lung tumor-associated polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of a lung tumor-associated polypeptide. Background level can be determined by various methods including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of, e.g., ⁹⁹Tc. The labeled binding molecule, e.g., antibody or antibody fragment, will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 7 to 10 days.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the binding molecule is labeled with a radioisotope and is detected in the patient using a radiation responsive surgical instrument (Thurston et al., U.S. Pat. No. 5,441,050). In another embodiment, the binding molecule is labeled with a fluorescent compound and is detected in the patient using a fluorescence responsive scanning instrument. In another embodiment, the binding molecule is labeled with a positron emitting metal and is detected in the patent using positron emission-tomography. In yet another embodiment, the binding molecule is labeled with a paramagnetic label and is detected in a patient using magnetic resonance imaging (MRI).

Antibody labels or markers for in vivo imaging of lung tumor-associated polypeptide expression include those detectable by X-radiography, nuclear magnetic resonance immaging (NMR), MRI, CAT-scans or electron spin resonance imaging (ESR). For X-radiography, suitable labels include radioisotopes such as barium or cesium, which emit detectable radiation but are not overtly harmful to the subject. Suitable markers for NMR and ESR. include those with a detectable characteristic spin, such as deuterium, which may be incorporated into the antibody by labeling of nutrients for the relevant hybridoma. Where in vivo imaging is used to detect enhanced levels of lung tumor-associated polypeptide expression for diagnosis in humans, it may be preferable to use human antibodies or “humanized” chimeric monoclonal antibodies. Such antibodies can be produced using techniques described herein or otherwise known in the art. For example methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

In a related embodiment to those described above, monitoring of an already diagnosed disease or disorder is carried out by repeating any one of the methods for diagnosing the disease or disorder, for example, one month after initial diagnosis, six months after initial diagnosis, one year after initial diagnosis, etc.

Where a diagnosis of a disorder, including diagnosis of a tumor, has already been made according to conventional methods, detection methods as disclosed herein are useful as a prognostic indicator, whereby patients continuing to exhibit enhanced lung tumor-associated polypeptide expression will experience a worse clinical outcome relative to patients whose expression level decreases nearer the standard level.

By “assaying the expression level of the lung tumor-associated polypeptide” is intended qualitatively or quantitatively measuring or estimating the level of the lung tumor-associated polypeptide in a first biological sample either directly (e.g., by determining or estimating absolute protein level) or relatively (e.g., by comparing to the tumor associated polypeptide level in a second biological sample). Preferably, lung tumor-associated polypeptide expression level in the first biological sample is measured or estimated and compared to a standard lung tumor-associated polypeptide level, the standard being taken from a second biological sample obtained from an individual not having the disorder or being determined by averaging levels from a population of individuals not having the disorder. As will be appreciated in the art, once the “standard” lung tumor-associated polypeptide level is known, it can be used repeatedly as a standard for comparison.

By “biological sample” is intended any biological sample obtained from an individual, cell line, tissue culture, or other source of cells potentially expressing the lung tumor-associated polypeptide. As indicated, biological samples include tissue sources which contain cells potentially expressing the lung tumor-associated polypeptide. Methods for obtaining tissue biopsies and body fluids from mammals are well known in the art.

In an additional embodiment, antibodies, or immunospecific fragments of antibodies directed to a conformational epitope of a lung tumor-associated polypeptide may be used to quantitatively or qualitatively detect the presence of lung tumor-associated polypeptides or conserved variants or peptide fragments thereof. This can be accomplished, for example, by immunofluorescence techniques employing a fluorescently labeled antibody coupled with light microscopic, flow cytometric, or fluorimetric detection.

Binding molecules for use in the diagnostic methods described above include any binding molecule which specifically binds to a lung tumor-associated polypeptide. Such polypeptides include, but are not limited to, a lung tumor-associated polypeptide comprising, consisting essentially of or consisting of a polypeptide selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID. NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51 and SEQ ID NO:52. Corresponding variant polypeptides comprising, consisting essentially of, or consisting of polypeptides which are at least 70%, 75%, 80%, 85%, 90%, 95%, or 100% identical to the lung tumor-associated polypeptides selected from the group consisting of SEQ ID NOs: 1 to 52 are also contemplated by the invention.

In the above embodiments, exemplary “fragments” of a lung tumor associate-polypeptide or variant polypeptide include but are not limited to: a fragment comprising, consisting essentially of, or consisting of a fragment selected from the group consisting of: SEQ ID NOs:13-24.

In the above embodiments, exemplary “fragments” of a lung tumor associate-polypeptide or variant polypeptide include but are not limited to: a fragment comprising, consisting essentially of, or consisting of a fragment selected from the group consisting of: amino acids 58-80 of SEQ ID NO:25; amino acids 137-369 of SEQ ID NO:25; amino acids 158-277 of SEQ ID NO:25; amino acids 173-299 of SEQ ID NO:25; amino acids 176-422 of SEQ ID NO:25; amino acids 205-283 of SEQ ID NO:25; amino acids 402-424 of SEQ ID NO:25; amino acids 100-216 of SEQ ID NO:26; amino acids 239-438 of SEQ ID NO:26; amino acids 453-529 of SEQ ID NO:26; amino acids 677-709 of SEQ ID NO:26; amino acids 65-88 of SEQ ID NO:27; amino acids 289-297 of SEQ ID NO:27; amino acids 321-370 of SEQ ID NO:27; amino acids 414-462 of SEQ ID NO:27; amino acids 418-429 of SEQ ID NO:27; amino acids 469-517 of SEQ ID NO:27; amino acids 473-486 of SEQ ID NO:27; amino acids 527-575 of SEQ ID NO:27; amino acids 533-549 of SEQ ID NO:27; amino acids 582-630 of SEQ ID NO:27; amino acids 634-695 of SEQ ID NO:27; amino acids 936-994 of SEQ ID NO:27; amino acids 982-1248 of SEQ ID NO:27; amino acids 993-1339 of SEQ ID NO:27. amino acids 1000-1247 of SEQ ID NO:27; amino acids 1005-1249 of SEQ ID NO:27; amino acids 1021-1243 of SEQ ID NO:27; amino acids 1023-1240 of SEQ ID NO:27; amino acids 1302-1541 of SEQ ID NO:27; amino acids 75-95 of SEQ ID NO:28; amino acids 50-291 of SEQ ID NO:29; amino acids 154-207 of SEQ ID NO:30; amino acids 211-469 of SEQ ID NO:30; amino acids 216-476 of SEQ ID NO:30; amino acids 229-419 of SEQ ID NO:30; amino acids 235-475 of SEQ ID NO:30; amino acids 253-455 of SEQ ID NO:30; amino acids 286-304 of SEQ ID NO:30; amino acids 291-482 of SEQ ID NO:30; amino acids 331-353 of SEQ ID NO:30; amino acids 485-496 of SEQ ID NO:30; amino acids 50-104 of SEQ ID NO:31; amino acids 111-161 of SEQ ID NO:31; amino acids 169-225 of SEQ ID NO:31; amino acids 232-340 of SEQ ID NO:31; amino acids 409-615 of SEQ ID NO:31; amino acids 1119-1293 of SEQ ID NO:31; amino acids 2252-2478 of SEQ ID NO:31; amino acids 2-122 of SEQ ID NO:32; amino acids 34-133 of SEQ ID NO:33; amino acids 35-133 of SEQ ID NO:33; amino acids 50-116 of SEQ ID NO:33; amino acids 138-172 of SEQ ID NO:33;amino acids 144-159 of SEQ ID NO:33; amino acids 151-211 of SEQ ID NO:33; amino acids 235-400 of SEQ ID NO:33; amino acids 242-330of SEQ ID NO:33; amino acids 249-320 of SEQ ID NO:33; amino acids 257-314 of SEQ ID NO:33; amino acids 293-307 of SEQ ID NO:33; amino acids 333-422 of SEQ ID NO:33; amino acids 347-406 of SEQ ID NO:33; amino acids 426-515 of SEQ ID NO:33; amino acids 441-499 of SEQ ID NO:33; amino acids 471-511 of SEQ ID NO:33; amino acids 517-609 of SEQ ID NO:33; amino acids 518-609 of SEQ ID NO:33; amino acids 532-593 of SEQ ID NO:33; amino acids 612-701 of SEQ ID NO:33; amino acids 714-800 of SEQ ID NO:33; amino acids 743-763 of SEQ ID NO:33; amino acids 812-907 of SEQ ID NO:33; amino acids 918-1005 of SEQ ID NO:33; amino acids 1017-1097 of SEQ ID NO:33; amino acids 1130-1148 of SEQ ID NO:33; amino acids 34-67 of SEQ ID NO:34; amino acids 234-261 of SEQ ID NO:35; amino acids 263-286 of SEQ ID NO:35; amino acids 287-308 of SEQ ID NO:35; amino acids 310-330 of SEQ ID NO:35; amino acids 334-357 of SEQ ID NO:35; amino acids 358-381 of SEQ ID NO:35; amino acids 383-405 of SEQ ID NO:35; amino acids 406-429 of SEQ ID NO:35; amino acids 430-453 of SEQ ID NO:35; amino acids 454-477 of SEQ ID NO:35; amino acids 478-501 of SEQ ID NO:35; amino acids 502-528 of SEQ ID NO:35; amino acids 502-525 of SEQ ID NO:35; amino acids 526-549 of SEQ ID NO:35; amino acids 539-552 of SEQ ID NO:35; amino acids 550-573 of SEQ ID NO:35; amino acids 550-563 of SEQ ID NO:35; amino acids 577-600 of SEQ ID NO:35; amino acids 601-624 of SEQ ID NO:35; amino acids 625-645 of SEQ ID NO:35; amino acids 625-654 of SEQ ID NO:35; amino acids 659-684 of SEQ ID NO:35; amino acids 689-790 of SEQ ID NO:35; amino acids 703-773 of SEQ ID NO:35; amino acids 793-884 of SEQ ID NO:35; amino acids 807-868 of SEQ ID NO:35; amino acids 867-883 of SEQ ID NO:35; amino acids 887-975 of SEQ ID NO:35; amino acids 897-974 of SEQ ID NO:35; amino acids 901-959 of SEQ ID NO:35; amino acids 74-114 of SEQ ID NO:36; amino acids 274-286 of SEQ ID NO:36; amino acids 277-299 of SEQ ID NO:36; amino acids 277-294 of SEQ ID NO:36; amino acids 277-287 of SEQ ID NO:36; amino acids 302-314 of SEQ ID NO:36; amino acids 305-327 of SEQ ID NO:36; amino acids 305-315 of SEQ ID NO:36; amino acids 307-348 of SEQ ID NO:36; amino acids 330-342 of SEQ ID NO:36; amino acids 332-340 of SEQ ID NO:36 amino acids 333-355 of SEQ ID NO:36; amino acids 333-343 of SEQ ID NO:36; amino acids 335-356 of SEQ ID NO:36; amino acids 358-370 of SEQ ID NO:36; amino acids 360-368 of SEQ ID NO:36; amino acids 361-383 of SEQ ID NO:36; amino acids 361-371 of SEQ ID NO:36; amino acids 386-398 of SEQ ID NO:36; amino acids 389-411 of SEQ ID NO:36; amino acids 389-399 of SEQ ID NO:36; amino acids 390-412 of SEQ ID NO:36; amino acids 391-412 of SEQ ID NO:36; amino acids 402-440 of SEQ ID NO:36; amino acids 414-426 of SEQ ID NO:36; amino acids 416-424 of SEQ ID NO:36; amino acids 417-439 of SEQ ID NO:36; amino acids 417-427 of SEQ ID NO:36; amino acids 417-440 of SEQ ID NO:36; amino acids 442-455 of SEQ ID NO:36; amino acids 444-452 of SEQ ID NO:36; amino acids 445-467 of SEQ ID NO:36; amino acids 445-455 of SEQ ID NO:36; amino acids 719-727 of SEQ ID NO:37; amino acids 1255-1340 of SEQ ID NO:37; amino acids 1341-1456 of SEQ ID NO:37; amino acids 1362-1389 of SEQ ID NO:37; amino acids 1470-1642 of SEQ ID NO:37; amino acids 1476-1487 of SEQ ID NO:37; amino acids 1644-1662 of SEQ ID NO:37; amino acids 1675-1684 of SEQ ID NO:37; amino acids 1824-1859 of SEQ ID NO:37; amino acids 1885-1896 of SEQ ID NO:37; amino acids 1910-1944 of SEQ ID NO:37; amino acids 1928-1943 of SEQ ID NO:37; amino acids 2112-2147 of SEQ ID NO:37; amino acids 2121-2161 of SEQ ID NO:37; amino acids 1-422 of SEQ ID NO:39; amino acids 77-87 of SEQ ID NO:39; amino acids 309-322 of SEQ ID NO:39; amino acids 507-537 of SEQ ID NO:39; amino acids 519-538 of SEQ ID NO:39; amino acids 591-602 of SEQ ID NO:39; amino acids 1-16 of SEQ ID NO:40; amino acids 28-139 of SEQ ID NO:40; amino acids 121-141 of SEQ ID NO:40; amino acids 149-264 of SEQ ID NO:40; amino acids 292-424 of SEQ ID NO:40; amino acids 449-589 of SEQ ID NO:40; amino acids 646-802 of SEQ ID NO:40; amino acids 141-274 of SEQ ID NO:41; amino acids 141-271 of SEQ ID NO:41; amino acids 290-325 of SEQ ID NO:41; amino acids 402-546 of SEQ ID NO:41; amino acids 402-543 of SEQ ID NO:41; amino acids 605-753 of SEQ ID NO:41; amino acids 605-750 of SEQ ID NO:41; amino acids 778-800 of SEQ ID NO:41; amino acids 844-974 of SEQ ID NO:41; amino acids 890-925 of SEQ ID NO:41; amino acids 1030-1161 of SEQ ID NO:41; amino acids 1030-1158 of SEQ ID NO:41; amino acids 1184-1216 of SEQ ID NO:41; amino acids 1253-1402 of SEQ ID NO:41; amino acids 129-157 of SEQ ID NO:42; amino acids 647-655 of SEQ ID NO:42; amino acids 39-49 of SEQ ID NO:43; amino acids 74-91 of SEQ ID NO:43; amino acids 264-281 of SEQ ID NO:43; amino acids 356-372 of SEQ ID NO:43; amino acids 71-110 of SEQ ID NO:44; amino acids 75-110 of SEQ ID NO:44; amino acids 158-193 of SEQ ID NO:44; amino acids 203-239 of SEQ ID NO:44; amino acids 312-347 of SEQ ID NO:44; amino acids 349-388 of SEQ ID NO:44; amino acids 390-427 of SEQ ID NO:44; amino acids 429-468 of SEQ ID NO:44; amino acids 433-468 of SEQ ID NO:44; amino acids 670-720 of SEQ ID NO:44; amino acids 727-774 of SEQ ID NO:44; amino acids 783-830 of SEQ ID NO:44; amino acids 835-944 of SEQ ID NO:44; amino acids 4-516 of SEQ ID NO:45; amino acids 21-199 of SEQ ID NO:45; amino acids 37-418 of SEQ ID NO:45; amino acids 101-115 of SEQ ID NO:45; amino acids 110-268 of SEQ ID NO:45; amino acids 320-534 of SEQ ID NO:45; amino acids 321-596 of SEQ ID NO:45; amino acids 353-591 of SEQ ID NO:45; amino acids 438-456 of SEQ ID NO:45; amino acids 26-50 of SEQ ID NO:46; amino acids 35-457 of SEQ ID NO: 46; amino acids 62-468 of SEQ ID NO: 46; amino acids 74-534 of SEQ ID NO: 46; amino acids 324-496 of SEQ ID NO: 46; amino acids 364-378 of SEQ ID NO: 46; amino acids 441-470 of SEQ ID NO: 46; amino acids 552-565 of SEQ ID NO: 46; amino acids 145-156 of SEQ ID NO:47; amino acids 240-251 of SEQ ID NO:47; amino acids 287-298 of SEQ ID NO:47; amino acids 328-339 of SEQ ID NO:47; amino acids 377-388 of SEQ ID NO:47; 428-439 of SEQ ID NO:47; 506-517 of SEQ ID NO:47; amino acids 548-559 of SEQ ID NO:47; amino acids 506-525 of SEQ ID NO:48; amino acids 529-556 of SEQ ID NO:48; amino acids 604-626 of SEQ ID NO:48; amino acids 627-651 of SEQ ID NO:48;amino acids 652-674 of SEQ ID NO:48; amino acids 675-697 of SEQ ID NO:48; amino acids 698-720 of SEQ ID NO:48; amino acids 721-743 of SEQ ID NO:48; amino acids 744-766 of SEQ ID NO:48; amino acids 767-789 of SEQ ID NO:48; amino acids 51-95 of SEQ ID NO:49; amino acids 110-133 of SEQ ID NO:49; amino acids 134-157 of SEQ ID NO:49; amino acids 158-181 of SEQ ID NO:49; amino acids 182-205 of SEQ ID NO:49; amino acids 206-230 of SEQ ID NO:49; amino acids 231-254 of SEQ ID NO:49; amino acids 244-261 of SEQ ID NO:49; amino acids 371-381 of SEQ ID NO:49; amino acids 388-409 of SEQ ID NO:49; amino acids 429-446 of SEQ ID NO:49; amino acids 455-477 of SEQ ID NO:49; amino acids 478-499 of SEQ ID NO:49; amino acids 503-526 of SEQ ID NO:49; amino acids 527-551 of SEQ ID NO:49; amino acids 541-556 of SEQ ID NO:49; amino acids 552-575 of SEQ ID NO:49; amino acids 576-599 of SEQ ID NO:49; amino acids 576-602 of SEQ ID NO:49; amino acids 600-630 of SEQ ID NO:49; amino acids 661-683 of SEQ ID NO:49; amino acids 684-704 of SEQ ID NO:49; amino acids 731-869 of SEQ ID NO:49; amino acids 27-433 of SEQ ID NO:50; amino acids 104-154 of SEQ ID NO:52; amino acids 174-186 of SEQ ID NO:52; amino acids 226-279 of SEQ ID NO:52; amino acids 227-276 of SEQ ID NO:52; amino acids 227-223 of SEQ ID NO:52; or amino acids 273-282 of SEQ ID NO:52.

Additionally, exemplary fragments of the lung tumor-associated polypeptides and variant polypeptides include, but are not limited to, fragments of the extracellular domains of the lung tumor-associated polypeptides described herein. For example, fragments selected from the group consisting of: amino acids 90-458 of SEQ ID NO:25; amino acids 1-735 of SEQ ID NO:26; amino acids 1-1003 of SEQ ID NO:27; amino acids 1061-1064 of SEQ ID NO:27; amino acids 1129-1147 of SEQ ID NO:27; amino 1640 of SEQ ID NO:27; amino acids 33-36 of SEQ ID NO:28; amino acids 81-112 of SEQ ID NO:29; 171-173 of SEQ ID NO:29; 271-315 of SEQ ID NO:29; amino acids 1-219 of SEQ ID NO:30; amino acids 278-282 of SEQ ID NO:30; amino acids 348-380 of SEQ ID NO:30; amino acids 447-449 of SEQ ID NO:30; amino acids 1-2163 of SEQ ID NO:31; amino acids 2221-2239 of SEQ ID NO:31; amino acids 2306-2332 of SEQ ID NO:31; amino acids 2483-2639 of SEQ ID NO:31; amino acids 40-130 of SEQ ID NO:32; amino acids 1-1120 of SEQ ID NO:33; amino acids 35-248 of SEQ ID NO:34; amino acids 1-986 of SEQ ID NO:35; amino acids 28-473 of SEQ ID NO:36; amino acids 1-2159 of SEQ ID NO:37; amino acids 1-119 of SEQ ID NO:38; amino acids 1-506 of SEQ ID NO:39; amino acids 1-867 of SEQ ID NO:40; amino acids 1-1719 of SEQ ID NO:41; amino acids 53-693 of SEQ ID NO:42; amino acids 1-66 of SEQ ID NO:43; amino acids 331-349 of SEQ ID NO:43; amino acids 394-407 of SEQ ID NO:43; amino acids 456-626 of SEQ ID NO:43; amino acids 59-1025 of SEQ ID NO:44; amino acids 1-19 of SEQ ID NO:45; amino acids 69-95 of SEQ ID NO:45; amino acids 159-167 of SEQ ID NO:45; amino acids 226-244 of SEQ ID NO.45; amino acids 342-350 of SEQ ID NO:45; amino acids 409-422 of SEQ ID NO:45; amino acids 516-519 of SEQ ID NO:45; amino acids 1-71 of SEQ ID NO:46; amino acids 121-181 of SEQ ID NO:46; amino acids 235-248 of SEQ ID NO:46; amino acids 306-324 of SEQ ID NO:46; amino acids 395-413 of SEQ ID NO:46; amino acids 461-474 of SEQ ID NO:46; amino acids 533-725 of SEQ ID NO:46; amino acids 1-755 of SEQ ID NO:47; amino acids 1-43 of SEQ ID NO:48; amino acids 161-279 of SEQ ID NO:48; amino acids 346-821 of SEQ ID NO:48; amino acids 1-688 of SEQ ID NO:49; amino acids 31-44 of SEQ ID NO:50; amino acids 110-212 of SEQ ID NO:50; amino acids 263-387 of SEQ ID NO:50; amino acids 27-54 of SEQ ID NO:51; amino acids 1-50 of SEQ ID NO:52; amino acids 99-101 of SEQ ID NO:52; amino acids 163-176 of SEQ ID NO:52; and amino acids 326-328 of SEQ ID NO:52.

Corresponding variant polypeptides comprising, consisting essentially of, or consisting of polypeptides which are at least 70%, 75%, 80%, 85%, 90%, 95%, or 100% identical to the lung tumor-associated polypeptides selected from the group consisting of SEQ ID NOs: 1 to 52 are also contemplated by the invention.

Other binding molecules for use in the diagnostic methods described herein include binding molecules which specifically bind to at least one epitope of a lung tumor-associated polypeptide where the epitope comprises, consists essentially of, or consists of at least about four to five amino acids amino acids of a lung tumor-associated polypeptide, at least seven, at least nine, or between at least about 15 to about 30 amino acids of a lung tumor-associated polypeptide. The amino acids of a given epitope of a lung tumor-associated polypeptide as described may be, but need not be contiguous. In certain embodiments, the at least one epitope of a lung tumor-associated polypeptide comprises, consists essentially of, or consists of a non-linear epitope formed by the extracellular domain of a lung tumor-associated polypeptide as expressed on the surface of a cell. Thus, in certain embodiments the at least one epitope of a lung tumor-associated polypeptide comprises, consists essentially of, or consists of at least 4, at least 5, at least 6, at least 7, at least 8, at least 9, at least 10, at least 11, at least 12, at least 13, at least 14, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of a lung tumor-associated polypeptide, where non-contiguous amino acids form an epitope through protein folding.

Additional binding molecules include those which specifically bind to at least one epitope of a lung tumor-associated polypeptide, where the epitope comprises, consists essentially of, or consists of, in addition to one, two, three, four, five, six or more contiguous or non-contiguous amino acids of a lung tumor-associated polypeptide as described above, an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

Cancers that may be diagnosed, and/or prognosed using the methods described above include but are not limited to, colorectal cancer, breast cancer, ovarian cancer, prostate cancer, pancreatic cancer, lung cancer, liver cancer, uterine cancer, and/or skin cancer.

### ANTIBODIES OR IMMUNOSPECIFIC FRAGMENTS THEREOF

In one embodiment a binding molecule for use in the methods of the invention is an antibody molecule, or immunospecific fragment thereof. Unless it is specifically noted, as used herein a “fragment thereof” in reference to an antibody refers to an immunospecific fragment, i.e., an antigen-specific fragment. In one embodiment, a binding molecule, e.g., an antibody of the invention is a bispecific binding molecule, binding polypeptide, or antibody, e.g., a bispecific antibody, minibody, domain deleted antibody, or fusion protein having binding specificity for more than one epitope, e.g., more than one antigen or more than one epitope on the same antigen. In one embodiment, a bispecific binding molecule, binding polypeptide, or antibody has at least one binding domain specific for at least one epitope on a target polypeptide disclosed herein, e.g., a lung tumor-associated polypeptide. In another embodiment, a bispecific binding molecule, binding polypeptide, or antibody has at least one binding domain specific for an epitope on a target polypeptide and at least one target binding domain specific for a drug or toxin. In yet another embodiment, a bispecific binding molecule, binding polypeptide, or antibody has at least one binding domain specific for an epitope on a lung tumor-associated polypeptide disclosed herein, and at least one binding domain specific for a prodrug. A bispecific binding molecule, binding polypeptide, or antibody may be a tetravalent antibody that has two target binding domains specific for an epitope of a target polypeptide disclosed herein and two target binding domains specific for a second target. Thus, a tetravalent bispecific binding molecule, binding polypeptide, or antibody may be bivalent for each specificity.

Antibody binding molecules for use in the treatment methods of the present invention, as known by those of ordinary skill in the art, can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain embodiments, methods of treating hyperproliferative diseases according to the present invention comprise administration of an antibody, or immunospecific fragment thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the C_{H}2 domain will be deleted.

In certain antibodies or immunospecific fragments thereof for use in the diagnostic and therapeutic methods described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of antibodies or immunospecific fragments thereof for use in the diagnostic and therapeutic methods disclosed herein can be made from whole precursor or parent antibodies using techniques known in the art. Exemplary techniques are discussed in more detail herein.

In certain embodiments both the variable and constant regions of lung tumor-associated polypeptide-specific antibodies or immunospecific fragments thereof for use in the diagnostic and treatment methods disclosed herein are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos.: 6,150,584; 6,458,592; 6,420,140. Other techniques are known in the art. Fully human antibodies can likewise be produced by various display technologies, e.g., phage display or other viral display systems, as described in more detail elsewhere herein.

Binding molecules, e.g., binding polypeptides, e.g., lung tumor-associated polypeptide-specific antibodies or immunospecific fragments thereof for use in the diagnostic and treatment methods disclosed herein can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

Binding molecules, e.g., binding polypeptides, e.g., lung tumor-associated polypeptide-specific antibodies or immunospecific fragments thereof for use in the diagnostic and treatment methods disclosed herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In preferred embodiments, a binding molecule, e.g., a binding polypeptide, e.g., a lung tumor-associated polypeptide-specific antibody or immunospecific fragment thereof for use in the diagnostic and treatment methods disclosed herein will not elicit a deleterious immune response in the animal to be treated, e.g., in a human. In one embodiment, binding molecules, e.g., binding polypeptides, e.g., lung tumor-associated polypeptide-specific antibodies or immunospecific fragments thereof for use in the diagnostic and treatment methods disclosed herein be modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536.(1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, V_{H} and V_{L} sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative V_{H} and V_{L} sequences are designed comprising combinations of amino acid substitutions and these sequences are subsequently incorporated into a range of binding polypeptides, e.g., lung tumor-associated polypeptide-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In the therapeutic methods described herein, administration is to an animal, e.g., a human, in need of treatment for cancer or other hyperproliferative disorder. For example, a binding molecule, e.g., a binding polypeptide, e.g., a lung tumor-associated polypeptide-specific antibody or immunospecific fragment thereof may be administered to a human patient diagnosed with a tumor, other cancerous lesion, or other hyperproliferative disorder, a human patient who has been treated for cancer and is in remission, but is in need of further chronic treatment to prevent recurrence or spread of cancer, a human who exhibits early warning signs for a certain cancer or hyperproliferative disorder and is a candidate for preventative treatment, or preventatively to a human who is genetically predisposed to contract a certain cancer.

The methods of treatment of hyperproliferative disorders as described herein are typically tested in vitro, and then in vivo in an acceptable animal model, for the desired therapeutic or prophylactic activity, prior to use in humans. Suitable animal models, including transgenic animals, are well known to those of ordinary skill in the art. For example, in vitro assays to demonstrate the therapeutic utility of binding molecule described herein include the effect of a binding molecule on a cell line or a patient tissue sample. The effect of the binding molecule on the cell line and/or tissue sample can be determined utilizing techniques known to those of skill in the art including, but not limited to, apoptosis assays and cell lysis assays. In accordance with the invention, in vitro assays which can be used to determine whether administration of a specific binding molecule is indicated, include in vitro cell culture assays in which a patient tissue sample is grown in culture, and exposed to or otherwise administered a compound, and the effect of such compound upon the tissue sample is observed.

Antibodies or fragments thereof for use as therapeutic binding molecules may be generated by any suitable method known in the art. Polyclonal antibodies to an antigen of interest can be produced by various procedures well known in the art. For example, a binding molecule, e.g., a binding polypeptide, e.g., a lung tumor-associated polypeptide-specific antibody or immunospecific fragment thereof can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art including the use of hybridoma and recombinant and phage display technology.

Using art recognized protocols, in one example, antibodies are raised in mammals by multiple subcutaneous or intraperitoneal injections of the relevant antigen (e.g., purified tumor associated antigens such as a lung tumor-associated polypeptides, varient polypeptides, frgaments thereof, or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

The polypeptide sequence of the lung tumor-associated polypeptides of the present invention was determined via mass spectroscopy. Accordingly, the source of antigen (e.g. a lung tumor-associated polypeptide, variant polypeptide, or fragment thereof may be prepared according to methods well known in the art. For example, the protein may be isolated from large amounts of the disease-associated tissue, smaller fragments of the lung tumor-associated polypeptide or variant polypeptide (about 10 to 125 amino acids) can be produced synthetically, the corresponding polynucleotide which encodes the lung tumor-associated polypeptide can also be isolated and cloned according to methods known in the art.

Finally, small polynucleotide fragments can be produced based on deducing the polynucleotide sequence from the amino acid sequence. For example a polynucleotide sequence which encoded a 12 amino acid fragment of a lung tumor-associated polypeptide could be synthesized based on the genetic code. Table 6 below indicates all of the bases which code for an amino acid. Thus, one of ordinary skill in the art could deduce a polynucleotide coding sequence based on the amino acid sequences described herein. The coding sequence could then be cloned into an expression vector described elsewhere herein and the resulting polypeptide could be purified, using methods known to one of ordinary skill in the art, see for example, the techniques described in “Methods In Enzymology” , 1990, Academic Press, Inc., San Diego, “Protein Purification: Principles and Practice” , 1982, Springer-Verlag, New York, which are incorporated by reference herein in their entirety. The purified polypeptide then could be used to immunize animals in the antibody production method described above. Additionally, the deduced polynucleotude sequences can be use to clone the polynucleotide which encodes the lung tumor-associated polypeptides described herein.

TABLE 6

The Standard Genetic Code

| | | | |
|---|---|---|---|
| T | C | A | G |

| | | | | | | | | |
|---|---|---|---|---|---|---|---|---|
| T | TTT | Phe (F) | TCT | Ser (S) | TAT | Tyr (Y) | TGT | Cys (C) |
| | TTC | ″ | TCC | ″ | TAC | ″ | TGC | |
| | TTA | Leu (L) | TCA | ″ | TAA | Ter | TGA | Ter |
| | TTG | ″ | TCG | ″ | TAG | Ter | TGG | Trp (W) |
| C | CTT | Leu (L) | CCT | Pro (P) | CAT | His (H) | CGT | Arg (R) |
| | CTC | ″ | CCC | ″ | CAC | ″ | CGC | ″ |
| | CTA | ″ | CCA | ″ | CAA | Gln (Q) | CGA | ″ |
| | CTG | ″ | CCG | ″ | CAG | ″ | CGG | ″ |
| A | ATT | Ile (I) | ACT | Thr (T) | AAT | Asn (N) | AGT | Ser (S) |
| | ATC | ″ | ACC | ″ | AAC | ″ | AGC | ″ |
| | ATA | ″ | ACA | ″ | AAA | Lys (K) | AGA | Arg (R) |
| | ATG | Met (M) | ACG | ″ | AAG | ″ | AGG | ″ |
| G | GTT | Val (V) | GCT | Ala (A) | GAT | Asp (D) | GGT | Gly (G) |
| | GTC | ″ | GCC | ″ | GAC | ″ | GGC | ″ |
| | GTA | ″ | GCA | ″ | GAA | Glu (E) | GGA | ″ |
| | GTG | ″ | GCG | ″ | GAG | ″ | GGG | ″ |

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a desired target polypeptide, e.g., a lung tumor-associated polypeptide.

Antibody fragments that recognize specific epitopes may be generated by known techniques. For example, Fab and F(ab′)2 fragments may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the C_{H}1 domain of the heavy chain.

Those skilled in the art will also appreciate that DNA encoding antibodies or antibody fragments (e.g., antigen binding sites) may also be derived from antibody phage libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. In particular, DNA sequences encoding V_{H} and V_{L} regions are amplified from animal cDNA libraries (e.g., human or murine cDNA libraries of lymphoid tissues) or synthetic cDNA libraries. In certain embodiments, the DNA encoding the V_{H} and V_{L} regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} or V_{L} regions are usually recombinantly fused to either the phage gene III or gene VIII. Phage expressing an antigen binding domain that binds to an antigen of interest (i.e., a lung tumor-associated polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Humanized antibodies are antibody molecules that bind the desired antigen having one or more complementarity determining regions (CDRs) from a non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988)). See also, U.S. Pat. No. 5,565,332.

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7 (5):437-444 (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture of antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., a lung tumor-associated polypeptide. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E. coli may also be used (Skerra et al., Science 242:1038-1041 (1988)).

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the V_{H} and V_{L} genes can be amplified using, e.g., RT-PCR. The V_{H} and V_{L} genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies for use in the diagnostic and therapeutic methods disclosed herein can be produced by any method known in the art for the synthesis of antibodies, in particular, by chemical synthesis or preferably, by recombinant expression techniques as described herein.

It will further be appreciated that the scope of this invention further encompasses all alleles, variants and mutations of antigen binding DNA sequences.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Recombinant expression of an antibody, or fragment, derivative or analog thereof, e.g., a heavy or light chain of an antibody which binds to a target molecule described herein, e.g., a lung tumor-associated polypeptide, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds). Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002 0123057 A1.

In one embodiment, a binding molecule or antigen binding molecule for use in the methods of the invention comprises a synthetic constant region wherein one or more domains are partially or entirely deleted (“domain-deleted antibodies”). In certain embodiments compatible modified antibodies will comprise domain deleted constructs or variants wherein the entire C_{H}2 domain has been removed (ΔC_{H}2 constructs). For other embodiments a short connecting peptide may be substituted for the deleted domain to provide flexibility and freedom of movement for the variable region. Those skilled in the art will appreciate that such constructs are particularly preferred due to the regulatory properties of the C_{H}2 domain on the catabolic rate of the antibody.

In certain embodiments, modified antibodies for use in the methods disclosed herein are minibodies. Minibodies can be made using methods described in the art (see, e.g., see e.g., U.S. Pat. No. 5,837,821 or WO 94/09817A1).

In another embodiment, modified antibodies for use in the methods disclosed herein are C_{H}2 domain deleted antibodies which are known in the art. Domain deleted constructs can be derived using a vector (e.g., from Biogen IDEC Incorporated) encoding an IgG₁, human constant domain (see, e.g., WO 02/060955A2 and W002/096948A2). This exemplary vector was engineered to delete the C_{H}2 domain and provide a synthetic vector expressing a domain deleted IgG₁, constant region.

In one embodiment, a binding molecule, e.g., a binding polypeptide, e.g., a lung tumor-associated polypeptide-specific antibody or immunospecific fragment thereof for use in the diagnostic and treatment methods disclosed herein comprises an immunoglobulin heavy chain having deletion or substitution of a few or even a single amino acid as long as it permits association between the monomeric subunits. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides the use of antibodies that comprise, consist essentially of, or consist of, variants (including derivatives) of antibody molecules (e.g., the V_{H} regions and/or V_{L} regions) described herein, which antibodies or fragments thereof immunospecifically bind to a lung tumor-associated polypeptide, variant polypeptide or fragment thereof. Standard techniques known to those of skill in the art can be used to introduce mutations in the nucleotide sequence encoding a binding molecule, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V_{H} region, V_{H}CDR1, V_{H}CDR2, V_{H}CDR3, V_{L} region, V_{L}CDR1, V_{L}CDR2, or V_{L}CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a lung tumor-associated polypeptide).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of a lung tumor-associated polypeptide) can be determined using techniques described herein or by routinely modifying techniques known in the art.

### FUSION PROTEINS AND ANTIBODY CONJUGATES

As discussed in more detail elsewhere herein, binding molecules, e.g., binding polypeptides, e.g., lung tumor-associated polypeptide-specific antibodies or immunospecific fragments thereof for use in the diagnostic and treatment methods disclosed herein may further be recombinantly fused to a heterologous polypeptide at the N- or C-terminus or chemically conjugated (including covalent and non-covalent conjugations) to polypeptides or other compositions. For example, lung tumor-associated polypeptide-specific binding molecules may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Binding molecules, e.g., binding polypeptides, e.g., lung tumor-associated polypeptide-specific antibodies or immunospecific fragments thereof for use in the diagnostic and treatment methods disclosed herein include derivatives that are modified, i.e., by the covalent attachment of any type of molecule to the antibody such that covalent attachment does not prevent the antibody binding a lung tumor-associated polypeptide. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Binding molecules, e.g., binding polypeptides, e.g., lung tumor-associated polypeptide-specific antibodies or immunospecific fragments thereof for use in the diagnostic and treatment methods disclosed herein can be composed of amino acids joined to each other by peptide bonds or modified peptide bonds, i.e., peptide isosteres, and may contain amino acids other than the 20 gene-encoded amino acids. Lung tumor-associated polypeptide-specific antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the lung tumor-associated polypeptide-specific antibody, including the peptide backbone, the amino acid side-chains and the amino or carboxyl termini, or on moieties such as carbohydrates. It will be appreciated that the same type of modification may be present in the same or varying degrees at several sites in a given lung tumor-associated polypeptide-specific antibody. Also, a given lung tumor-associated polypeptide-specific antibody may contain many types of modifications. Lung tumor-associated polypeptide-specific antibodies may be branched, for example, as a result of ubiquitination, and they may be cyclic, with or without branching. Cyclic, branched, and branched cyclic lung tumor-associated polypeptide-specific antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising, consisting essentially of, or consisting of, an antibody (including molecules comprising, consisting essentially of, or consisting of, antibody fragments or variants thereof), that immunospecifically binds to a lung tumor-associated polypeptide, and a heterologous polypeptide. Preferably, the heterologous polypeptide to which the antibody is fused is useful for function or is useful to target the lung tumor-associated polypeptide expressing cells, including but not limited to a breast, ovarian, bladder, colon, lung, prostate, and pancreatic cancer cell. In an alternative preferred embodiment, the heterologous polypeptide to which the antibody is fused is useful for T cell, macrophage, and/or monocyte cell function or is useful to target the antibody to a T cell, macrophage, or monocyte. In one embodiment, a fusion protein of the invention comprises, consists essentially of, or consists of, a polypeptide having the amino acid sequence of any one or more of the V_{H} regions of an antibody of the invention or the amino acid sequence of any one or more of the V_{L} regions of an antibody of the invention or fragments or variants thereof, and a heterologous polypeptide sequence. In another embodiment, a fusion protein for use in the diagnostic and treatment methods disclosed herein comprises, consists essentially of, or consists of a polypeptide having the amino acid sequence of any one, two, three, or more of the V_{H} CDRs of an lung tumor-associated polypeptide-specific antibody, or the amino acid sequence of any one, two, three, or more of the V_{L} CDRs of a lung tumor-associated polypeptide-specific antibody, or fragments or variants thereof, and a heterologous polypeptide sequence. In one embodiment, the fusion protein comprises, consists essentially of, or consists of a polypeptide having the amino acid sequence of a V_{H} CDR3 of an lung tumor-associated polypeptide-specific antibody, or fragment or variant thereof, and a heterologous polypeptide sequence, which fusion protein specifically binds to at least one epitope of lung tumor-associated polypeptide. In another embodiment, a fusion protein comprises, consists essentially of, or consists of a polypeptide having the amino acid sequence of at least one V_{H} region of a lung tumor-associated polypeptide-specific antibody and the amino acid sequence of at least one V_{L} region of a lung tumor-associated polypeptide-specific antibody or immunospecific fragments thereof, and a heterologous polypeptide sequence. Preferably, the V_{H} and V_{L} regions of the fusion protein correspond to a single source antibody (or scFv or Fab fragment) which specifically binds at least one epitope of a lung tumor-associated polypeptide. In yet another embodiment, a fusion protein for use in the diagnostic and treatment methods disclosed herein comprises, consists essentially of, or consists of a polypeptide having the amino acid sequence of any one, two, three or more of the V_{H} CDRs of a lung tumor-associated polypeptide-specific antibody and the amino acid sequence of any one, two, three or more of the V_{L} CDRs of a lung tumor-associated polypeptide-specific antibody, or fragments or variants thereof, and a heterologous polypeptide sequence. Preferably, two, three, four, five, six, or more of the V_{H}CDR(s) or V_{L}CDR(s) correspond to single source antibody (or scFv or Fab fragment) of the invention. Nucleic acid molecules encoding these fusion proteins are also encompassed by the invention.

The invention also pertains to the use of binding molecules which comprise one or more immunoglobulin domains. Fusion proteins for use in the diagnostic and therapeutic methods disclosed herein comprise a binding domain (which comprises at least one binding site) and a dimerization domain (which comprises at least one heavy chain portion). The subject fusion proteins may be bispecific (with one binding site for a first target and a second binding site for a second target) or may be multivalent (with two binding sites for the same target).

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

In one embodiment a fusion protein combines the binding domain(s) of the ligand or receptor (e.g. the extracellular domain (ECD) of a receptor) with at least one heavy chain domain and a synthetic connecting peptide. In one embodiment, when preparing the fusion proteins of the present invention, nucleic acid encoding the binding domain of the ligand or receptor domain will be fused C-terminally to nucleic acid encoding the N-terminus of an immunoglobulin constant domain sequence. N-terminal fusions are also possible. In one embodiment, a fusion protein includes a C_{H}2 and a C_{H}3 domain. Fusions may also be made to the C-terminus of the Fc portion of a constant domain, or immediately N-terminal to the C_{H}1 of the heavy chain or the corresponding region of the light chain.

In one embodiment, the sequence of the ligand or receptor binding domain is fused to the N-terminus of the Fc domain of an immunoglobulin molecule. It is also possible to fuse the entire heavy chain constant region to the ligand or receptor binding domain sequence. In one embodiment, a sequence beginning in the hinge region just upstream of the papain cleavage site which defines IgG Fc chemically (i.e. residue 216, taking the first residue of heavy chain constant region to be 114 according to the Kabat system), or analogous sites of other immunoglobulins is used in the fusion. The precise site at which the fusion is made is not critical; particular sites are well known and may be selected in order to optimize the biological activity, secretion, or binding characteristics of the molecule. Methods for making fusion proteins are known in the art.

For bispecific fusion proteins, the fusion proteins can be assembled as multimers, and particularly as heterodimers or heterotetramers. Generally, these assembled immunoglobulin-like proteins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

As discussed elsewhere herein, binding molecules, e.g., binding polypeptides, e.g., lung tumor-associated polypeptide-specific antibodies or immunospecific fragments thereof for use in the diagnostic and treatment methods disclosed herein may be fused to heterologous polypeptides to increase the in vivo half life of the polypeptides or for use in immunoassays using methods known in the art. In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5 receptor, have been fused with Fc portions for the purpose of high-throughput screening assays to identify antagonists of hIL-5. (See, D. Bennett et al., J. Molecular Recognition 8:52-58 (1995); K. Johanson et al. J. Biol. Chem. 270:9459-9471 (1995).

Moreover, binding molecules, e.g., binding polypeptides, e.g., lung tumor-associated polypeptide-specific antibodies or immunospecific fragments thereof for use in the diagnostic and treatment methods disclosed herein can be fused to marker sequences, such as a peptide to facilitate their purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). Ordinarily, the ligand or ligand binding partner is fused C-terminally to the N-terminus of the constant region of the heavy chain (or heavy chain portion) and in place of the variable region. Any transmembrane regions or lipid or phospholipid anchor recognition sequences of ligand binding receptor are preferably inactivated or deleted prior to fusion. DNA encoding the ligand or ligand binding partner is cleaved by a restriction enzyme at or proximal to the 5′ and 3′ ends of the DNA encoding the desired ORF segment. The resultant DNA fragment is then readily inserted into DNA encoding a heavy chain constant region. The precise site at which the fusion is made may be selected empirically to optimize the secretion or binding characteristics of the soluble fusion protein. DNA encoding the fusion protein is then transfected into a host cell for expression.

Binding molecules for use in the methods of the present invention may be used in non-conjugated form or may be conjugated to at least one of a variety of molecules, e.g., to improve the therapeutic properties of the molecule, to facilitate target detection, or for imaging or therapy of the patient. Binding molecules, e.g., binding polypeptides, e.g., lung tumor-associated polypeptide-specific antibodies or immunospecific fragments thereof for use in the diagnostic and treatment methods disclosed herein can be labeled or conjugated either before or after purification, when purification is performed.

In particular, binding molecules, e.g., binding polypeptides, e.g., lung tumor-associated polypeptide-specific antibodies or immunospecific fragments thereof for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG. In another embodiment, a binding molecule, e.g., a binding polypeptide, e.g., a lung tumor-associated polypeptide-specific antibody or immunospecific fragment thereof for use in the diagnostic and treatment methods disclosed herein can be conjugated to a molecule that decreases vascularization of tumors. In other embodiments, the disclosed compositions may comprise binding molecules, e.g., binding polypeptides, e.g., lung tumor-associated polypeptide-specific antibodies or immunospecific fragments thereof coupled to drugs or prodrugs. Still other embodiments of the present invention comprise the use of binding molecules, e.g., binding polypeptides, e.g., lung tumor-associated polypeptide-specific antibodies or immunospecific fragments thereof conjugated to specific biotoxins or their cytotoxic fragments such as ricin, gelonin, pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding molecules, e.g., binding polypeptides, e.g., lung tumor-associated polypeptide-specific antibodies or immunospecific fragments thereof may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Binding molecules, e.g., binding polypeptides, e.g., lung tumor-associated polypeptide-specific antibodies or immunospecific fragments thereof for use in the diagnostic and treatment methods disclosed herein may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane- 1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation U.S. Pat. Nos. 6,682,134, 6,399,061, and 5,843,439, incorporated herein by reference in their entireties, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, binding molecules may be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the can readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Those skilled in the art will appreciate that non-radioactive conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably. fluorescein-isothiocyanate. Conjugates of the binding molecules, e.g., binding polypeptides, e.g., lung tumor-associated polypeptide-specific antibodies or immunospecific fragments thereof with cytostatic/cytotoxic substances and metal chelates are prepared in an analogous manner.

Additional preferred agents for conjugation to binding molecules, e.g., binding polypeptides, e.g., lung tumor-associated polypeptide-specific antibodies or immunospecific fragments thereof are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the maytansinoid family of drugs. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. One skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins for preparation of conjugates may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that binding molecules, e.g., binding polypeptides, e.g., lung tumor-associated polypeptide-specific antibodies or immunospecific fragments thereof for use in the diagnostic and treatment methods disclosed herein can also be associated with or conjugated to a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the binding molecules, e.g., binding polypeptides, e.g., lung tumor-associated polypeptide-specific antibodies or immunospecific fragments thereof disclosed herein comprise cytokines such as lymphokines and interferons. In view of the instant disclosure it is submitted that one skilled in the art could readily form such constructs using conventional techniques.

Another class of compatible cytotoxins that may be used in association with or conjugated to the disclosed binding molecules, e.g., binding polypeptides, e.g., lung tumor-associated polypeptide-specific antibodies or immunospecific fragments thereof, are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked binding molecules of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In certain embodiments, a moiety that enhances the stability or efficacy of a binding molecule, e.g., a binding polypeptide, e.g., a lung tumor-associated polypeptide-specific antibody or immunospecific fragment thereof can be conjugated. For example, in one embodiment, PEG can be conjugated to the binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

The present invention further encompasses the use of binding molecules, e.g., binding polypeptides, e.g., lung tumor-associated polypeptide-specific antibodies or immunospecific fragments conjugated to a diagnostic or therapeutic agent. The binding molecules can be used diagnostically to, for example, monitor the development or progression of a tumor as part of a clinical testing procedure to, e.g., determine the efficacy of a given treatment and/or prevention regimen. Detection can be facilitated by coupling the binding molecule, e.g., binding polypeptide, e.g., lung tumor-associated polypeptide-specific antibody or immunospecific fragment thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

A binding molecule, e.g., a binding polypeptide, e.g., a lung tumor-associated polypeptide-specific antibody or immunospecific fragment thereof also can be detectably labeled by coupling it to a chemiluminescent compound. The presence of the chemiluminescent-tagged binding molecule is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which a binding molecule, e.g., a binding polypeptide, e.g., a lung tumor-associated polypeptide-specific antibody or immunospecific fragment thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enrymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the binding molecule will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the binding molecule, e.g., binding polypeptide, e.g., lung tumor-associated polypeptide-specific antibody or immunospecific fragment thereof, it is possible to detect cancer antigens through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

A binding molecule, e.g., a binding polypeptide, e.g., a lung tumor-associated polypeptide-specific antibody or immunospecific fragment thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to a binding molecule, e.g., a binding polypeptide, e.g., a lung tumor-associated polypeptide-specific antibody or immunospecific fragment thereof are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

### POLYNUCLEOTIDES ENCODING LUNG TUMOR ASSOCIATED POLYPEPTIDE-SPECIFIC BINDING MOLECULES

The present invention also provides for nucleic acid molecules encoding lung tumor associated polypeptide-specific antibodies or other binding molecules (including molecules comprising, consisting essentially of, or consisting of, antibody fragments or variants thereof).

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or other binding molecule may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody or other binding molecule, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a CDNA library that encodes the antibody or other binding molecule. Amplified nucleic acids generated by PCR may then be cloned into replicable cloning vectors using any method well known in the art.

Once the nucleotide sequence and corresponding amino acid sequence of the antibody or other binding molecule is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an lung tumor associated polypeptide-specific antibody or other binding molecule can be composed of any polyribonucleotide or polydeoxribonucleotide, which may be unmodified RNA or DNA or modified RNA or DNA. For example, a polynucleotide encoding a lung tumor associated polypeptide-specific antibody or other binding molecule can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding a lung tumor associated polypeptide-specific antibody can be composed of triple-stranded regions comprising RNA or DNA or both RNA and DNA. A polynucleotide encoding a lung tumor associated polypeptide-specific antibody or other binding molecule may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

### ANTIBODY EXPRESSION

Following manipulation of the isolated genetic material to provide binding molecules, e.g., binding polypeptides, e.g., lung tumor associated polypeptide-specific antibodies or immunospecific fragments thereof for use in the diagnostic and treatment methods disclosed herein, the polynucleotides encoding the binding molecules are typically inserted in an expression vector for introduction into host cells that may be used to produce the desired quantity of binding molecule.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes (preferably human) synthetic as discussed above. In one embodiment, this is effected using a proprietary expression vector of Biogen IDEC, Inc., referred to as NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the binding molecules, e.g., binding polypeptides, e.g., lung tumor associated polypeptide-specific antibodies or immunospecific fragments thereof for use in the diagnostic and treatment methods disclosed herein may be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of binding molecules, e.g., binding polypeptides, e.g., lung tumor associated polypeptide-specific antibodies or immunospecific fragments thereof in eukaryotic host cells. Compatible IRES sequences are disclosed in U.S. Pat. No. 6,193,980 which is also incorporated herein. Those skilled in the art will appreciate that such expression systems may be used to effectively produce the full range of binding molecules disclosed in the instant application.

More generally, once the vector or DNA sequence encoding a monomeric subunit of the binding polypeptide (e.g. a modified antibody) has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

Along those same lines, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3x 63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding binding molecules, e.g., binding polypeptides, e.g., lung tumor associated polypeptide-specific antibodies or immunospecific fragments thereof for use in the diagnostic and treatment methods disclosed herein can also be expressed non-mammalian cells such as bacteria or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trpl lesion as a characteristic of the yeast host cell genome then provides an effective environment for detecting transformation by growth in the absence of tryptophan.

### IMMUNOASSAYS

Binding molecules, e.g., binding polypeptides, e.g., lung tumor associated polypeptide-specific antibodies or immunospecific fragments thereof for use in the diagnostic and treatment methods disclosed herein may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitation a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32p or 1251) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

Lung tumor associated polypeptide-specific binding molecules may, additionally, be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a patient, and applying thereto a labeled lung tumor associated polypeptide-specific antibody or fragment thereof, preferably applied by overlaying the labeled antibody (or fragment) onto a biological sample. Through the use of such a procedure, it is possible to determine not only the presence of lung tumor associated protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for lung tumor associated polypeptide or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to lung tumor associated polypeptides or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled lung tumor associated polypeptide-specific antibody. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of lung tumor associated polypeptide-specific antibody may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE code No. BR-1001-86; BIAtechnology Handbook, version AB (reprinted 1998), BIACORE code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a monoclonal antibody. Epitope mapping with BIAcore, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BlAcore are straightforward to carry out.

For example, one can use a capture molecule to bind the first Mab, followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

### PHARMACEUTICAL COMPOSITIONS AND ADMINISTRATION METHODS

Methods of preparing and administering binding molecules, e.g., binding polypeptides, e.g., lung tumor-associated polypeptide-specific antibodies or immunospecific fragments thereof to a subject in need thereof are well known to or are readily determined by those skilled in the art. The route of administration of the binding molecule, e.g., binding polypeptide, e.g., lung tumor-associated polypeptide-specific antibody or immunospecific fragment thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules, e.g., binding polypeptides, e.g., lung tumor-associated polypeptide-specific antibodies or immunospecific fragments thereof can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule, e.g., a binding polypeptide, e.g., a lung tumor-associated polypeptide-specific antibody or immunospecific fragment thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampules, bags, bottles, syringes or vials, and sealed under aseptic conditions according to methods known in the art. Further, the preparations may be packaged and sold in the form of a kit such as those described in co-pending U.S.S.N. 09/259,337 (US-2002-0102208 A1), which is incorporated herein by reference in its entirety. Such articles of manufacture will preferably have labels or package inserts indicating that the associated compositions are useful for treating a subject suffering from, or predisposed to hyperproliferative disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or other binding molecule, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Binding molecules, e.g., binding polypeptides, e.g., lung tumor-associated polypeptide-specific antibodies or immunospecific fragments thereof for use in the diagnostic and treatment methods disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moeity, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules, e.g., binding polypeptides, e.g., lung tumor-associated polypeptide-specific antibodies or immunospecific fragments thereof for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding a binding molecule, e.g., a binding polypeptide, e.g., a lung tumor-associated polypeptide-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., Nucl. Acids Res. 16:3209 (1988), methylphosphonate oligonucleotides can be prepared by use of controlled pore glass polymer supports (Sarin et al., Proc. Natl. Acad. Sci. U.S.A. 85:7448-7451(1988)), etc.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where lung tumor-associated polypeptide-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

Agents of the invention can optionally be administered in combination with other agents that are effective in treating the disorder or condition in need of treatment (e.g., prophylactic or therapeutic).

Effective single treatment dosages (i.e., therapeutically effective amounts) of 90Y-labeled antibodies range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu,⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, 47Sc, ¹⁰⁵Rh, ¹⁰⁹Pd, ¹⁵³Sm, ¹⁸⁸Re, ¹⁹⁹Au, ²²⁵Ac,²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not binding molecules, e.g., binding polypeptides, e.g., lung tumor-associated polypeptide-specific antibodies or immunospecific fragments thereof for use in the diagnostic and treatment methods disclosed herein are used in a conjugated or unconjugated form, it will be appreciated that a major advantage of the present invention is the ability to use these molecules in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the molecules makes them particularly useful for treating patients that have reduced red marrow reserves and are sensitive to myelotoxicity. In this regard, the unique delivery profile of the molecules make them very effective for the administration of radiolabeled conjugates to myelosuppressed cancer patients. As such, binding molecules, e.g., binding polypeptides, e.g., lung tumor-associated polypeptide-specific antibodies or immunospecific fragments thereof for use in the treatment methods disclosed herein are useful in a conjugated or unconjugated form in patients that have previously undergone adjunct therapies such as external beam radiation or chemotherapy. In other preferred embodiments, binding molecules, e.g., binding polypeptides, e.g., lung tumor-associated polypeptide-specific antibodies or immunospecific fragments thereof (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies or other binding molecules and one or more chemotherapeutic agents. Particularly preferred embodiments of this aspect of the invention will comprise the administration of a radiolabeled binding polypeptide.

While binding molecules, e.g., binding polypeptides, e.g., lung tumor-associated polypeptide-specific antibodies or immunospecific fragments thereof may be administered as described immediately above, it must be emphasized that in other embodiments conjugated and unconjugated binding molecules may be administered to otherwise healthy patients as a first line therapeutic agent. In such embodiments binding molecules may be administered to patients having normal or average red marrow reserves and/or to patients that have not, and are not, undergoing adjunct therapies such as external beam radiation or chemotherapy.

However, as discussed above, selected embodiments of the invention comprise the administration of binding molecules, e.g., binding polypeptides, e.g., lung tumor-associated polypeptide-specific antibodies or immunospecific fragments thereof to myelosuppressed patients or in combination or conjunction with one or more adjunct therapies such as radiotherapy or chemotherapy (i.e. a combined therapeutic regimen). As used herein, the administration of binding molecules, e.g., binding polypeptides, e.g., lung tumor-associated polypeptide-specific antibodies or immunospecific fragments thereof in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates described herein. Conversely, cytotoxin-conjugated binding molecules could be administered intravenously followed by tumor localized external beam radiation. In yet other embodiments, binding molecules may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of a binding molecule (with or without cytotoxin) and the chemotherapeutic agent may be administered in any order and within any time frame that provides a therapeutic benefit to the patient. That is, the chemotherapeutic agent and binding molecule, e.g., binding polypeptide, e.g., lung tumor-associated polypeptide-specific antibody or immunospecific fragment thereof, may be administered in any order or concurrently. In selected embodiments binding molecules, e.g., binding polypeptides, e.g., lung tumor-associated polypeptide-specific antibodies or immunospecific fragments thereof for use in treatment methods disclosed herein will be administered to patients that have previously undergone chemotherapy. In yet other embodiments, binding molecules, e.g., binding polypeptides, e.g., lung tumor-associated polypeptide-specific antibodies or immunospecific fragments thereof for use in treatment methods disclosed herein will be administered substantially simultaneously or concurrently with the chemotherapeutic treatment. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the binding molecule will be administered within 4, 3, 2 or 1 week of any chemotherapeutic agent or treatment. In yet other embodiments the binding molecule will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppresion and one can easily measure the extent to which myelosuppresion is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated binding molecules, e.g., binding polypeptides, e.g., lung tumor-associated polypeptide-specific antibodies or immunospecific fragments thereof for use in treatment methods disclosed herein may be used to effectively treat patients having ANCs lower than about 2000/mm³ or platelet counts lower than about 150,000/mm^{3.} More preferably binding molecules, e.g., binding polypeptides, e.g., lung tumor-associated polypeptide-specific antibodies or immunospecific fragments thereof for use in treatment methods disclosed herein may be used to treat patients having ANCs of less than about 1500/mm³, less than about 1000/mm³ or even more preferably less than about 500/ mmd. Similarly, binding molecules, e.g., binding polypeptides, e.g., lung tumor-associated polypeptide-specific antibodies or immunospecific fragments thereof for use in treatment methods disclosed herein may be used to treat patients having a platelet count of less than about 75,000/mm³, less than about 50,000/mm³ or even less than about 10,000/mm^{3.} In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, the disclosed binding molecules, e.g., binding polypeptides, e.g., lung tumor-associated polypeptide-specific antibodies or immunospecific fragments thereof for use in treatment methods disclosed herein may be used to treat disorders in patients exhibiting myelosuppression regardless of the cause.

In this regard it will further be appreciated that binding molecules, e.g., binding polypeptides, e.g., lung tumor-associated polypeptide-specific antibodies or immunospecific fragments thereof for use in treatment methods disclosed herein may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other embodiments, radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medicine 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more binding molecules, e.g., binding polypeptides, e.g., lung tumor-associated polypeptide-specific antibodies or immunospecific fragments thereof as described herein.

Additional regimens that are useful in the context of the present invention include use of single alkylating agents such as cyclophosphamide or chlorambucil or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade malignancies, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the binding molecules disclosed herein may vary by subject or may be administered according to what is known in the art. See for example, Bruce A Chabner et al., Antineoplastic Agents, in Goodman & Gilman's The Pharmacological Basis of Therapeutics 1233-1287 ((Joel G. Hardman et al., eds., 9^{th} ed. (1996).

As previously discussed, binding molecules, e.g., binding polypeptides, e.g., lung tumor-associated polypeptide-specific antibodies or immunospecific fragments thereof, or recombinants thereof may be administered in a pharmaceutically effective amount for the in vivo treatment of mammalian hyperproliferative disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of binding molecule, e.g., binding polypeptide, e.g., lung tumor-associated polypeptide-specific antibody or immunospecific fragment thereof, or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the binding molecule will be preferably be capable of interacting with selected immunoreactive antigens on neoplastic or immunoreactive cells, or on non neoplastic cells, e.g., vascular cells associated with neoplastic cells and provide for an increase in the death of those cells. Of course, the pharmaceutical compositions of the present invention may be administered in single or multiple doses to provide for a pharmaceutically effective amount of the binding molecule.

In keeping with the scope of the present disclosure, binding molecules, e.g., binding polypeptides, e.g., lung tumor-associated polypeptide-specific antibodies or immunospecific fragments thereof for use in treatment methods disclosed herein may be administered to a human or other animal in accordance with the aforementioned methods of treatment in an amount sufficient to produce a therapeutic or prophylactic effect. The binding molecules, e.g., binding polypeptides, e.g., lung tumor-associated polypeptide-specific antibodies or immunospecific fragments thereof for use in treatment methods disclosed herein can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No: 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, NY (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W. H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984).

All of the references cited above, as well as all references cited herein, are incorporated herein by reference in their entireties.

### EXAMPLES

### Example 1—Preparation of Plasma Membranes

Tumor associated and normal lung tissue samples were washed twice with cold PBS. Cold homogenization buffer, HEES buffer (0.255 M sucrose, 1 mM EDTA, 1 mM EGTA, 10 mM HEPES, pH 7.4) supplemented with a protease-inhibitor cocktail (PIC; 0.1 mg/ml AEBSF, 2 μg/ml aprotinin, 40 μg/ml bestatin, 10 μg/ml chymostatin, 10 μg/ml E-64, 2 μg/ml leupeptin, 2 μg/ml Pepstatin A), was added and the samples were minced before being homogenized in an IKA Laboratories homogenizer. The homogenate was centrifuged to pellet debris and the supernatant was saved. This supernatant was then centrifuged at 16000g, and the resulting pellet was kept. The membrane pellet was resuspended in 1 ml of HEES+PIC buffer, and dispersed using a glass dounce homogenizer (5). The membranes were loaded on top of a 16 ml continuous gradient of 0-18% iodixanol in HEES buffer, and centrifuged at 130,000 g for 3 hr. Fractions (12×1.4 ml) were collected from the top of the gradient. The top four fractions (1.1 ml of each) were pooled, diluted with 8 ml of salt wash buffer (SWB; 0.15 M NaCl, 2 mM Mg(Cl)₂, 20 mM Tris-HCl, pH 7.5), and centrifuged at 100,000 g for 1 hr. The pellet was resuspended in 1 ml of SWB and centrifuged at 100,000 g for 30 min to yield the plasma membrane pellet. To analyze the gradient fractionation, 0.3 ml of each fraction was diluted with 0.7 ml SWB and centrifuged at 100,000 g for 30 min. The membrane pellets were dissolved in SDS-gel sample buffer.

### Example 2—Analytical Methods

Solubilized plasma membrane preparations, prepared as described in Example 1, were diluted into SDS loading buffer and 100 μg of protein, as determined by Bradford assay, was loaded on a 1.0 mm thick 8-12% Tris SDS-PAGE gel. The protein mixture was electrophoretically separated and then stained with Commasee Blue protein dye. The gels were destained with 50% ACN/water and cut into 18 sections according to a predetermined grid using the molecular weight markers of an adjacent lane. The sections were further destained, the proteins reduced with DTT and alkylated with iodoacetic acid. Typsin was added and the proteins in-gel digested overnight. The resulting tryptic peptides were extracted from the gel matrix with 1% formic acid, the volume adjusted to either 100 μL and placed into a reversed phase high-pressure chromotography (HPLC) vial for separation followed by tandem mass spectrometry-microsequencing (MS/MS) in an Agilent MSD Ion Trap mass spectrometer.

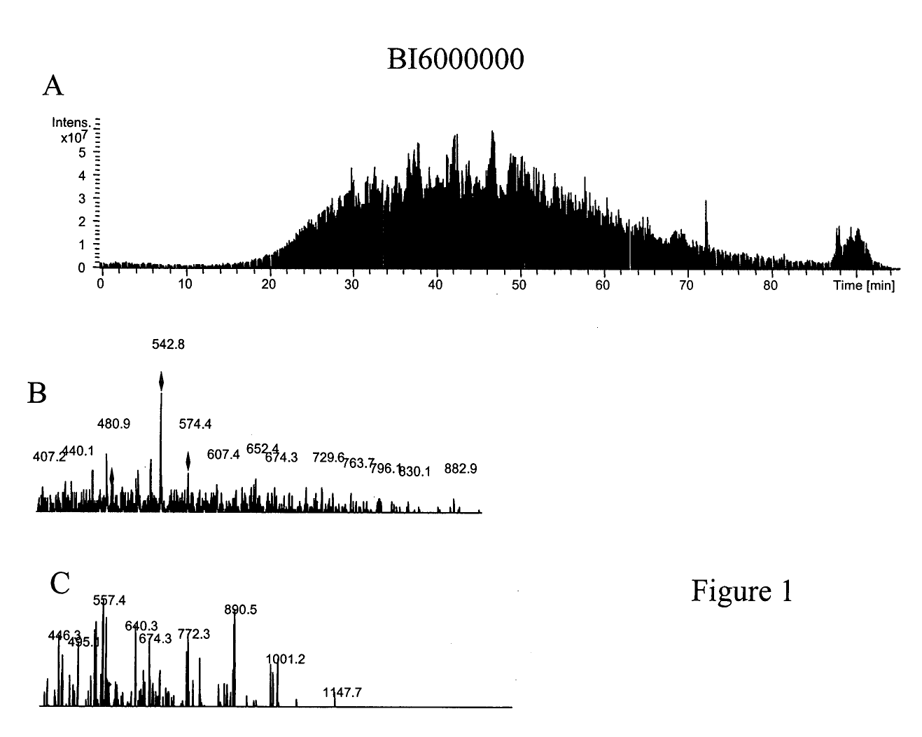

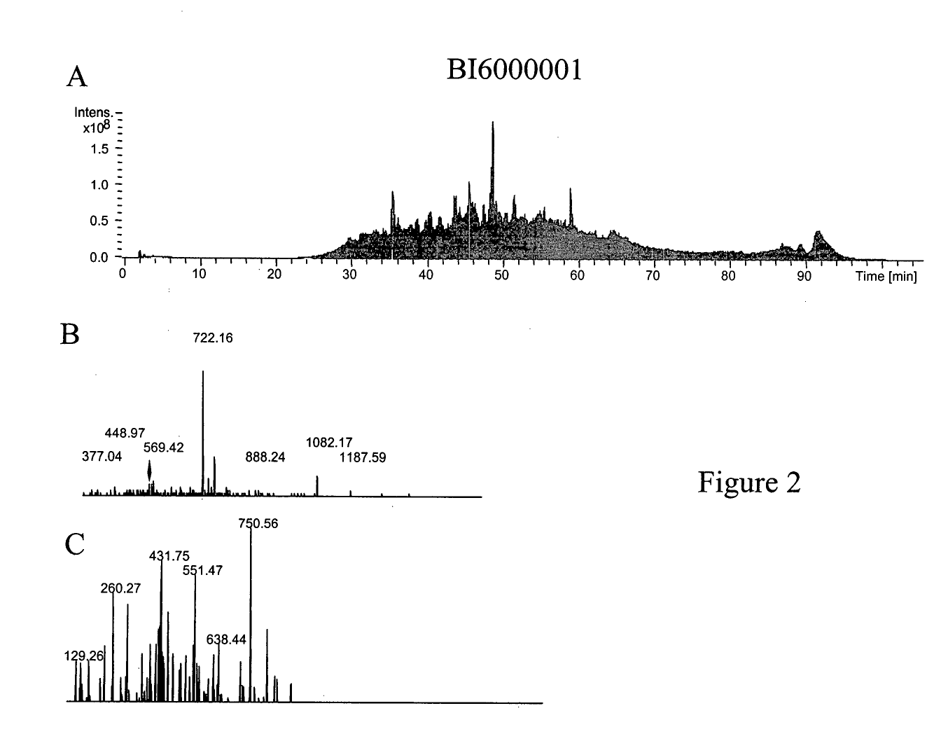

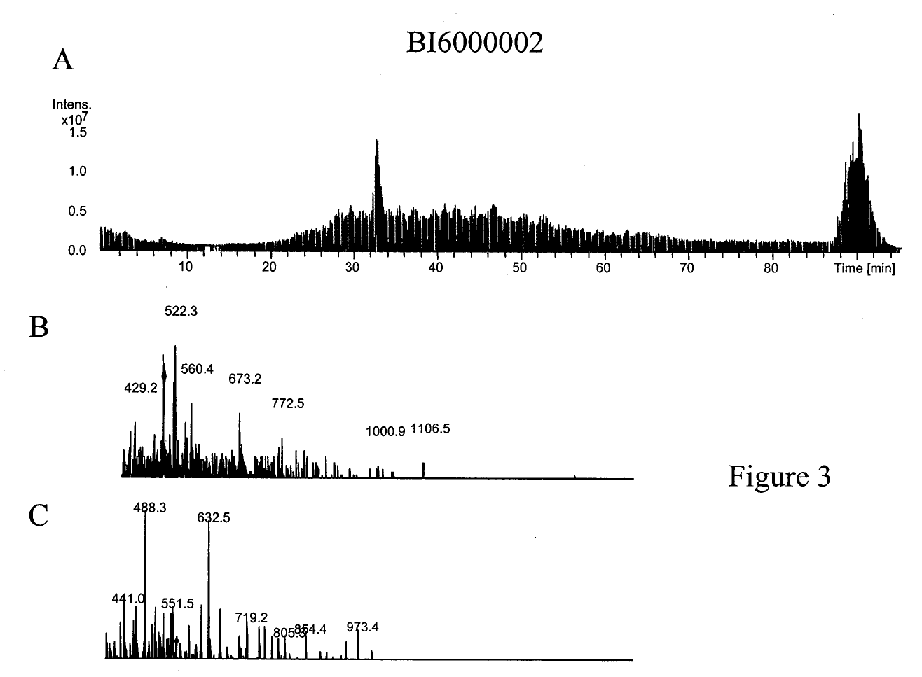

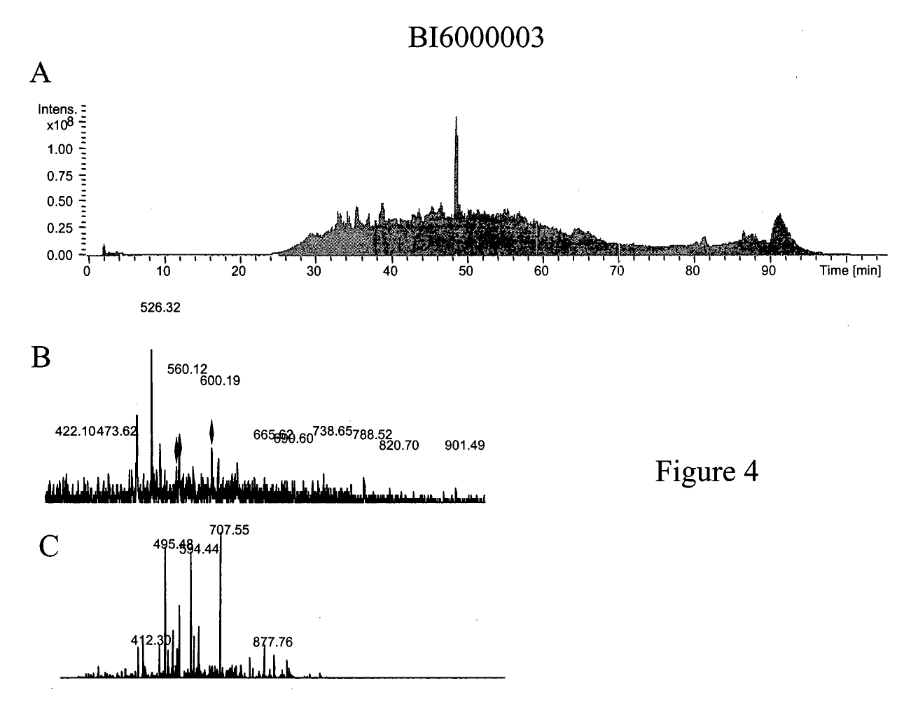

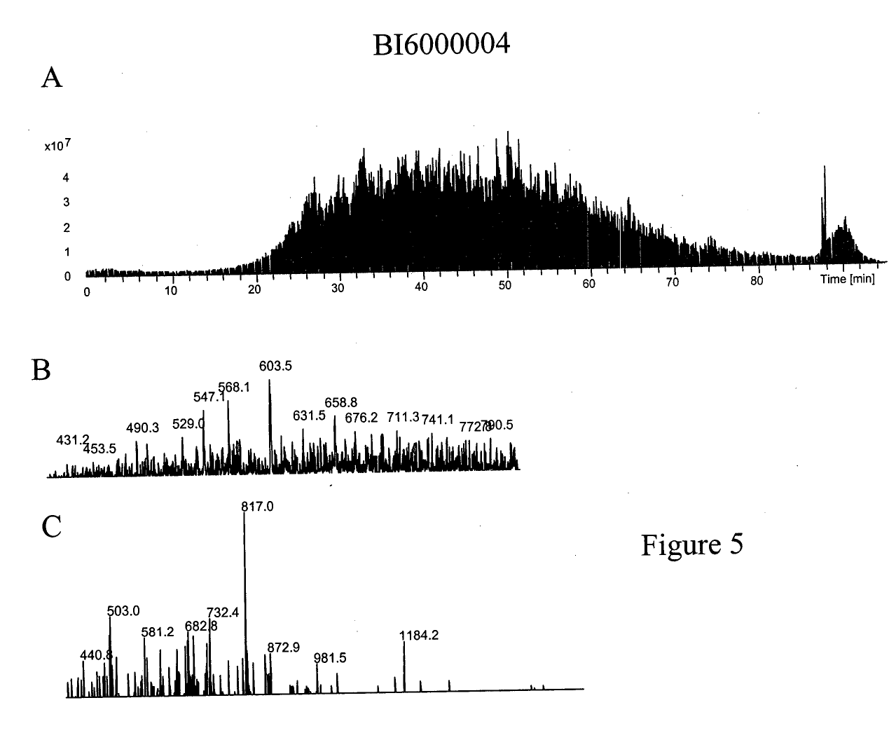

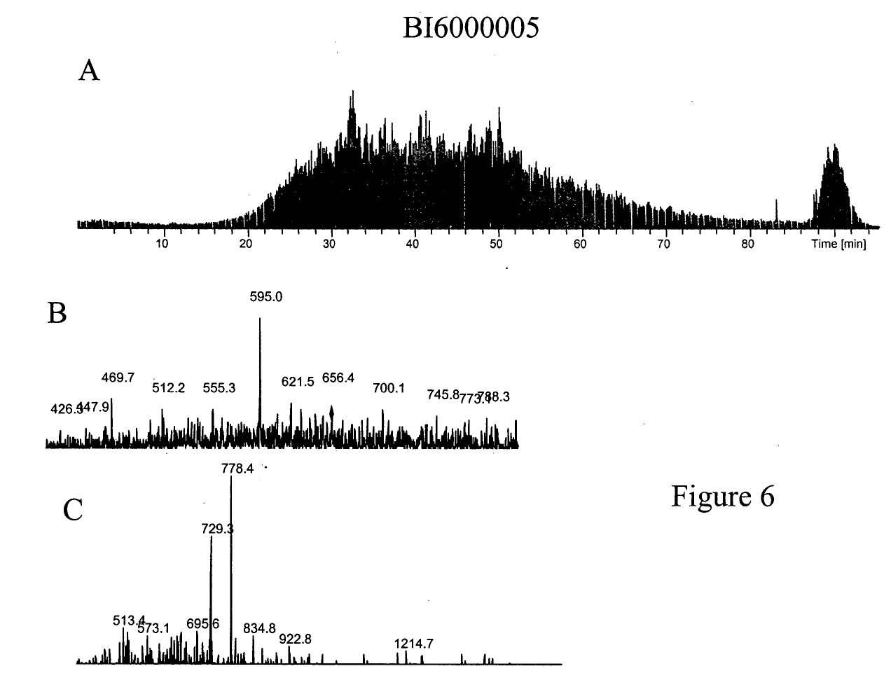

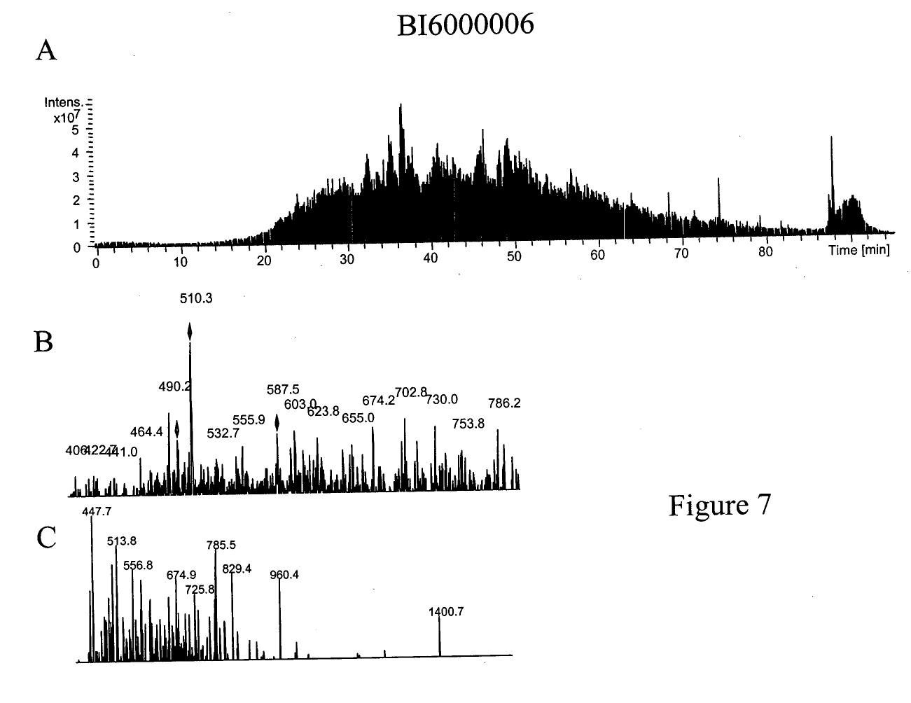

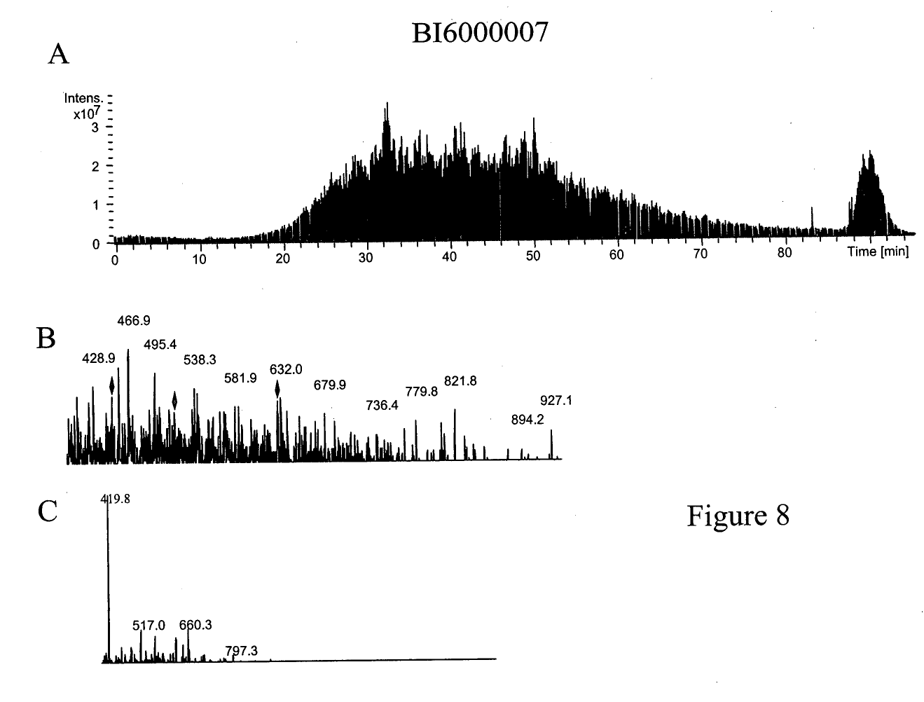

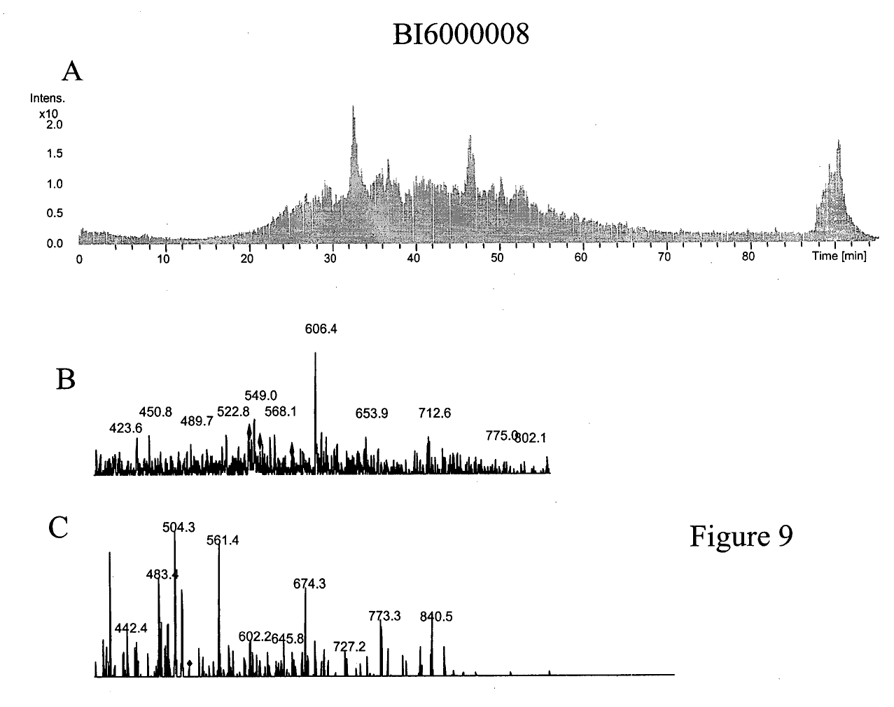

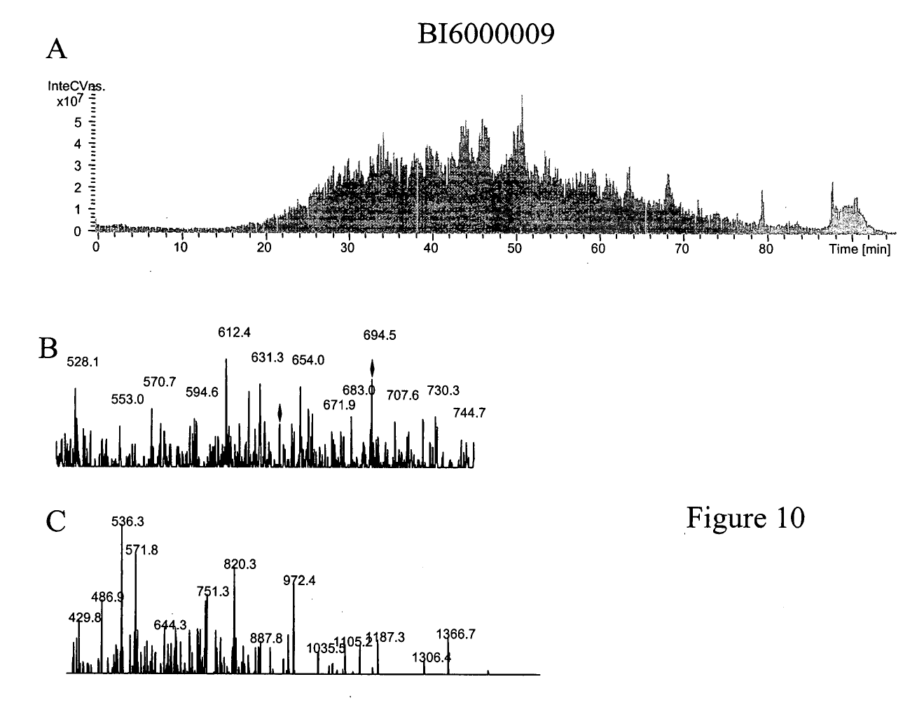

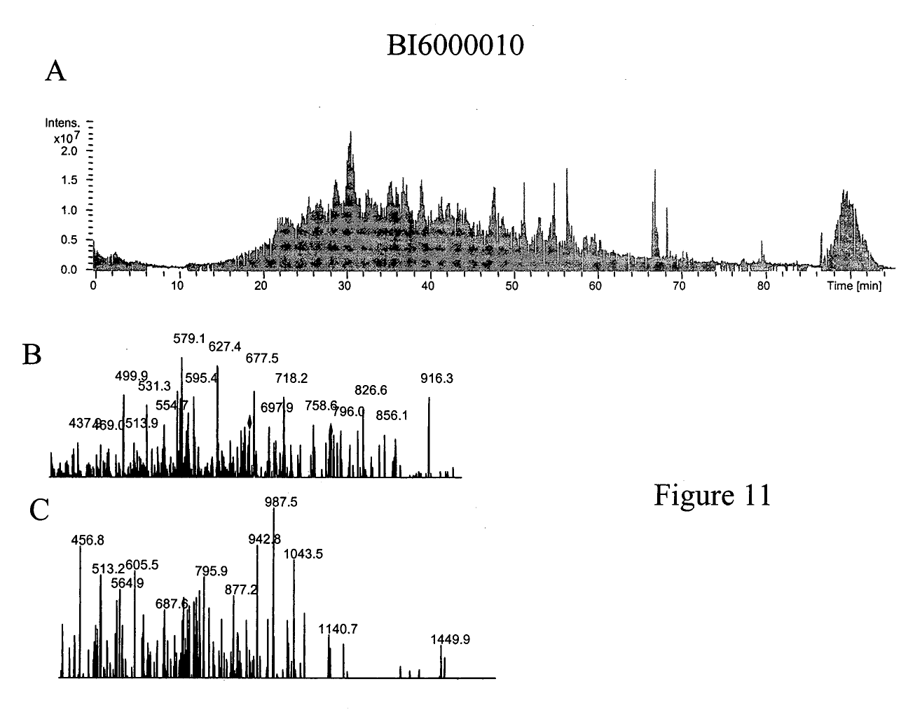

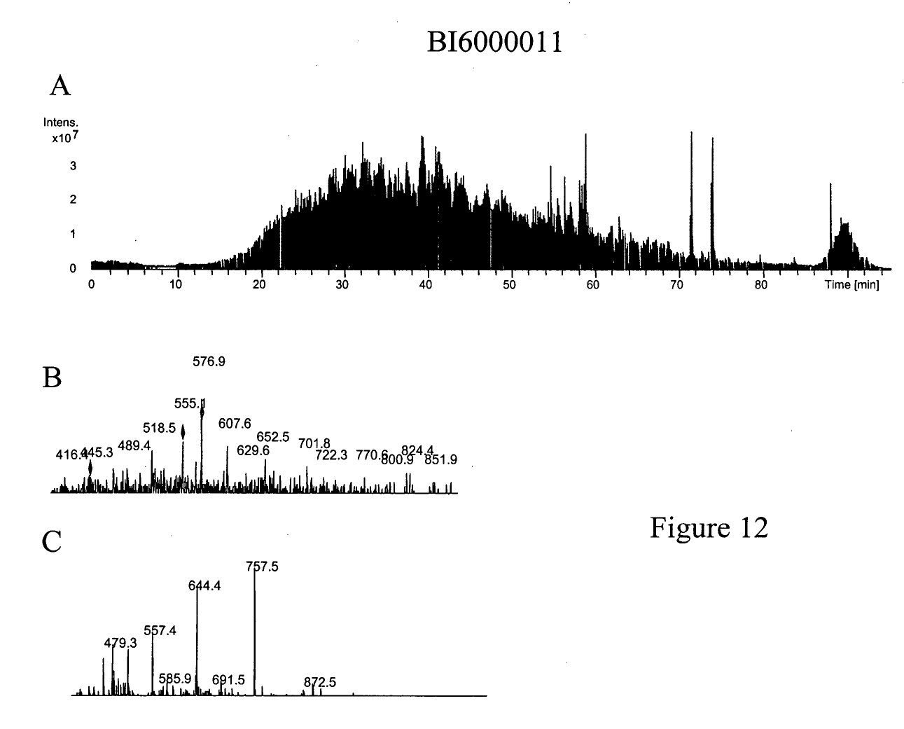

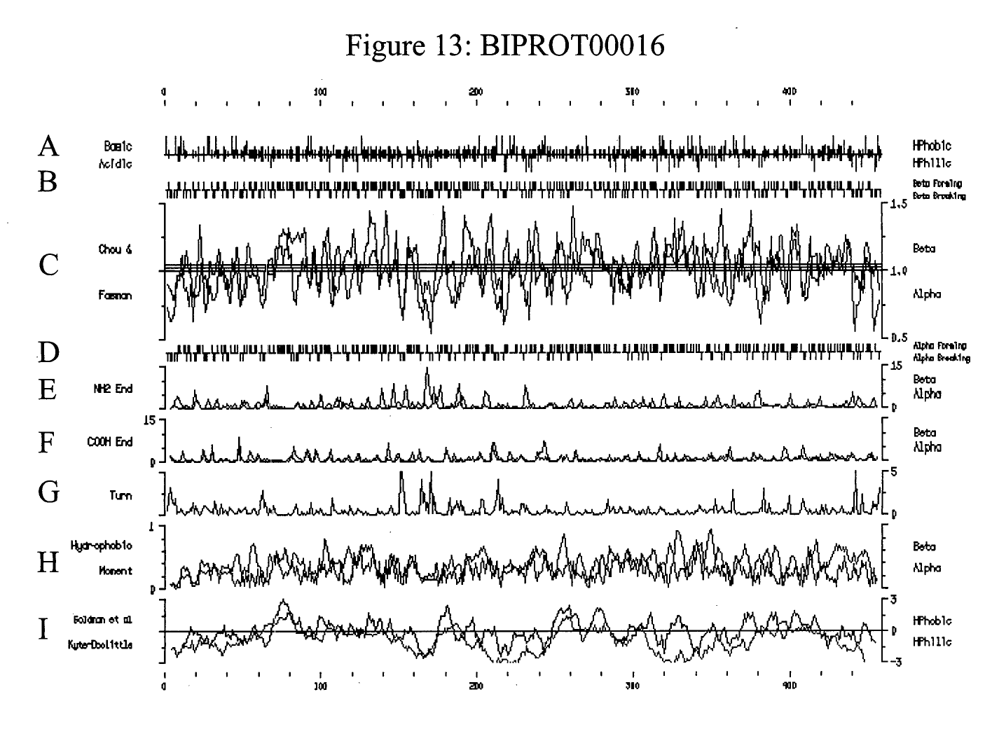

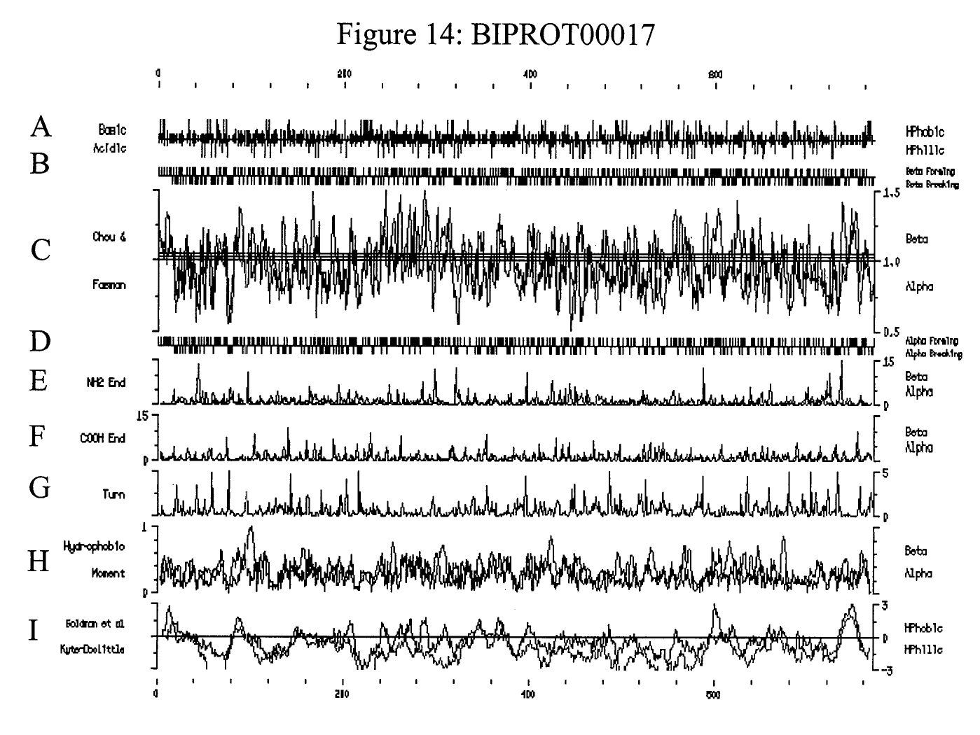

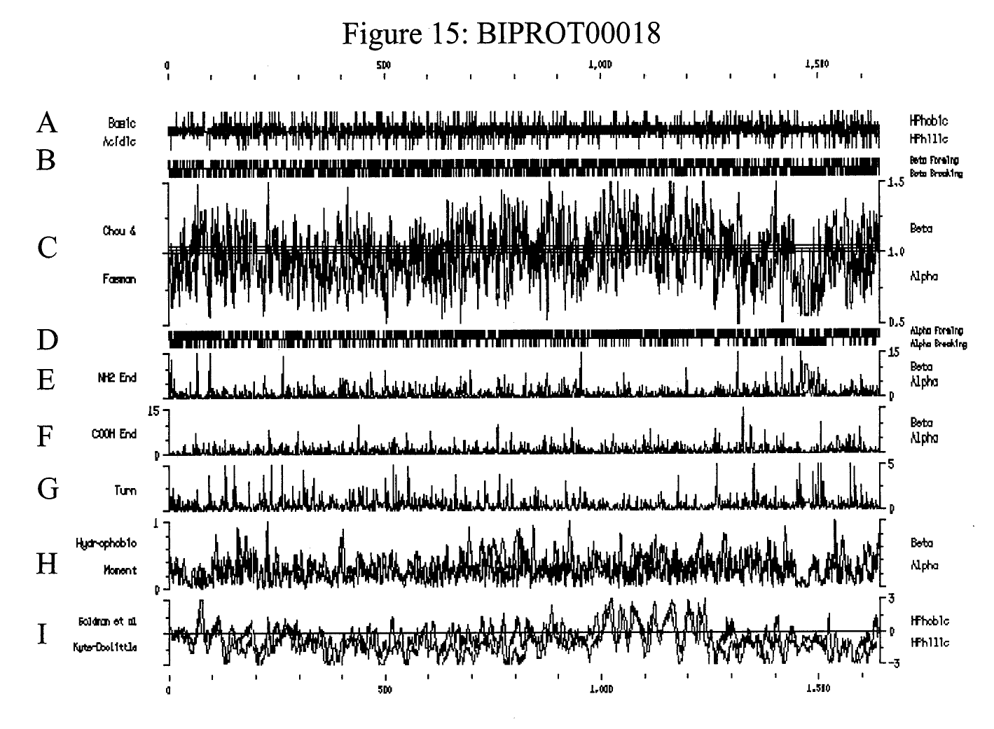

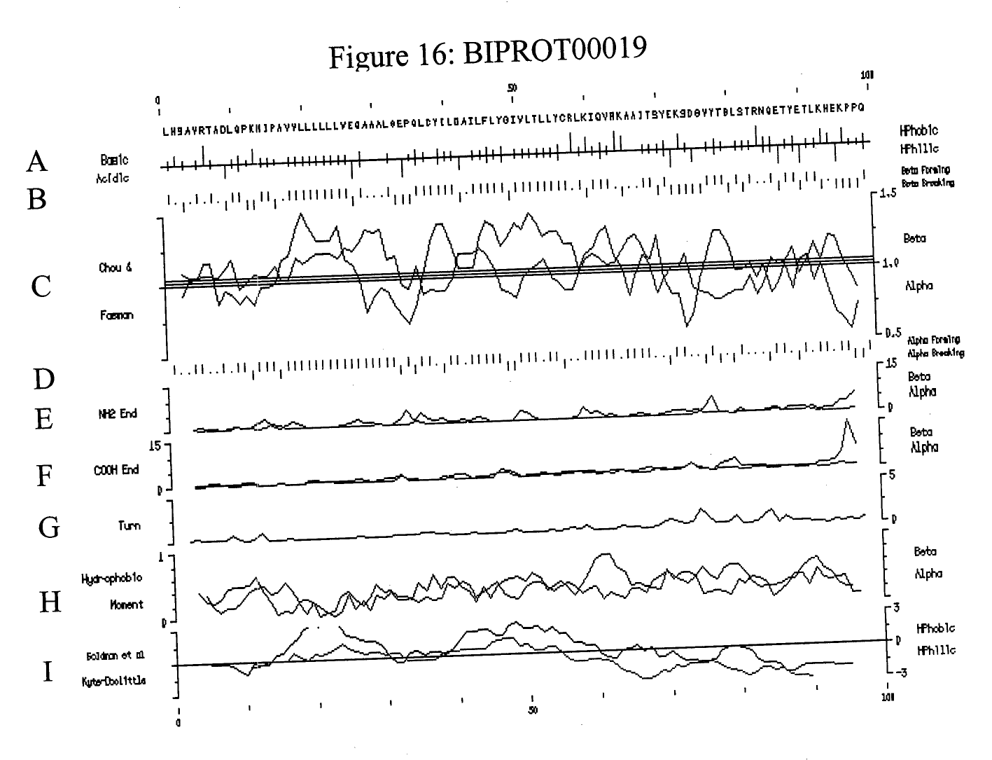

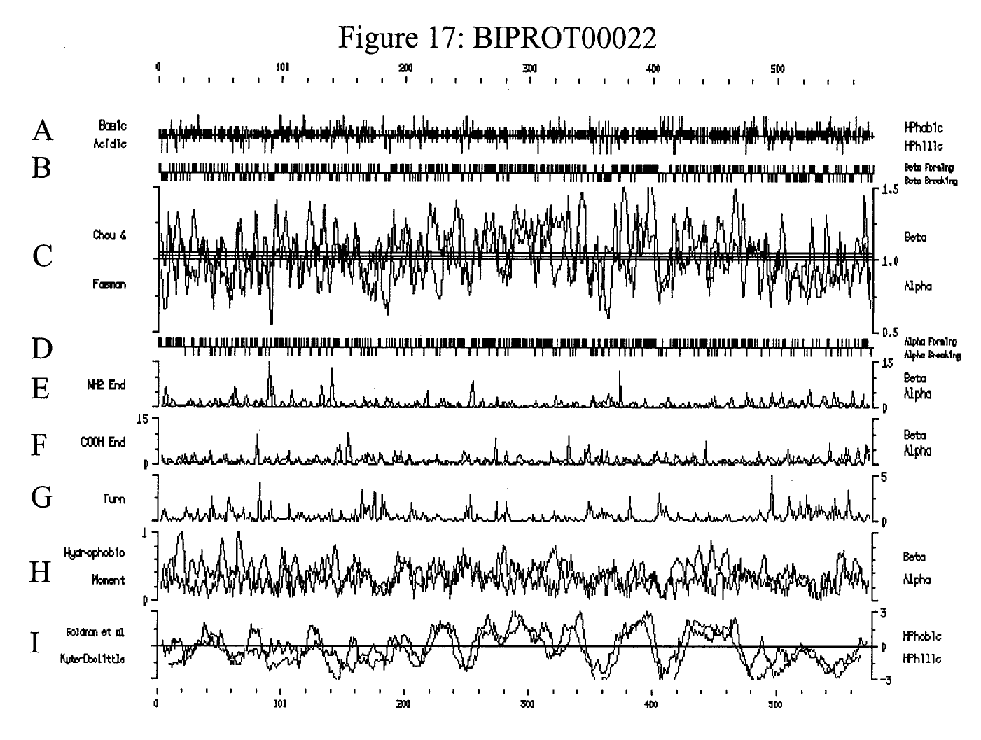

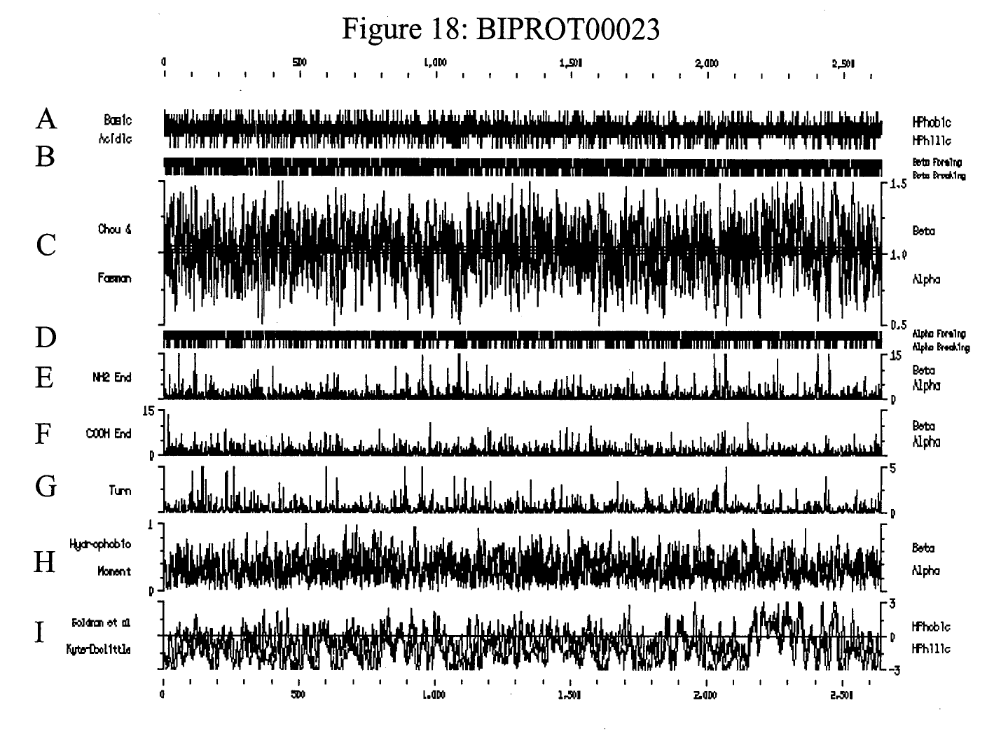

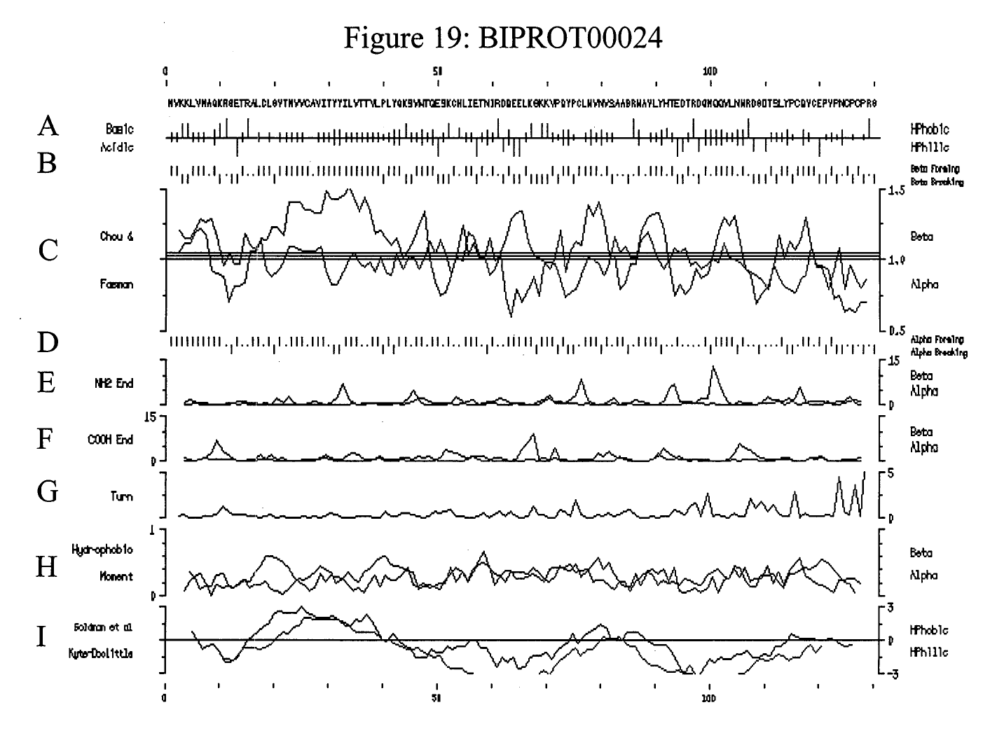

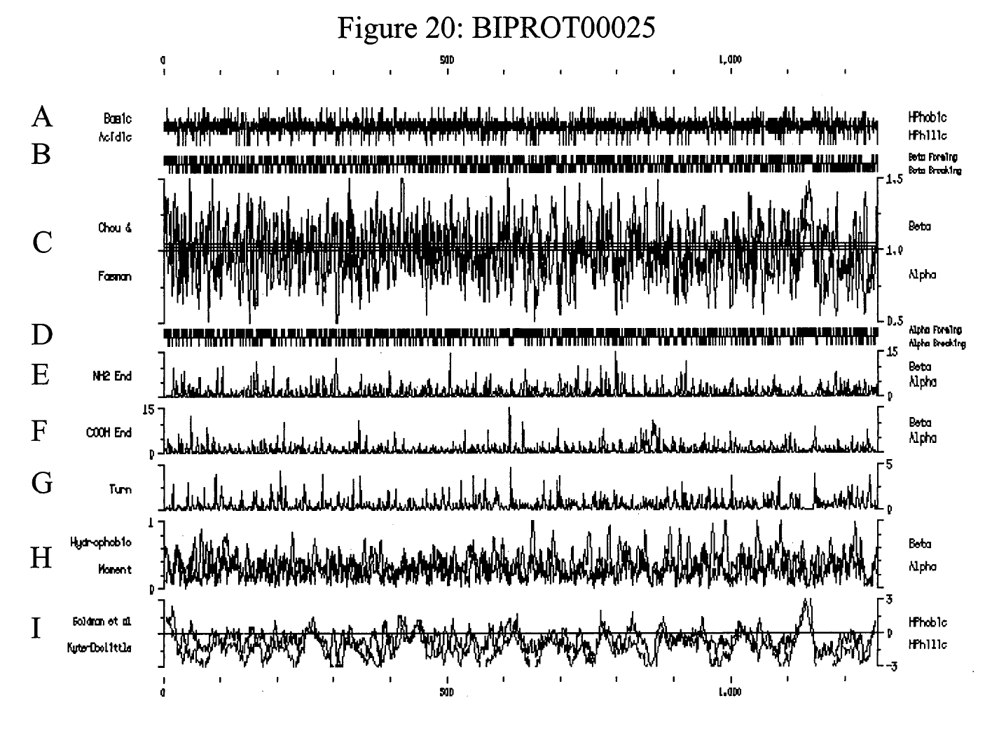

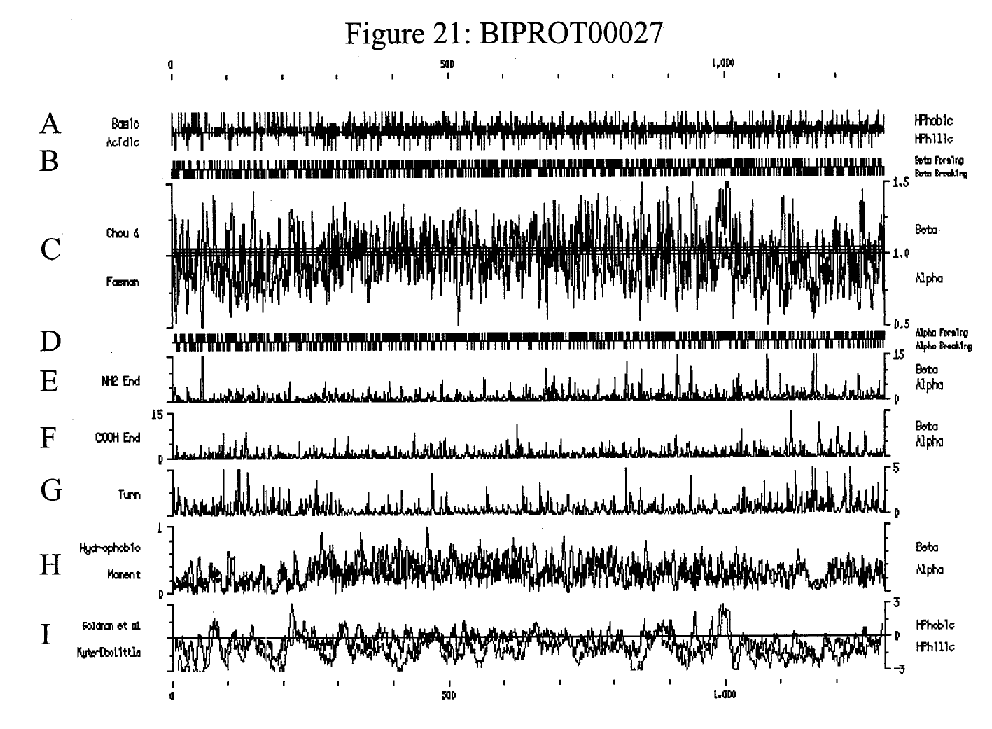

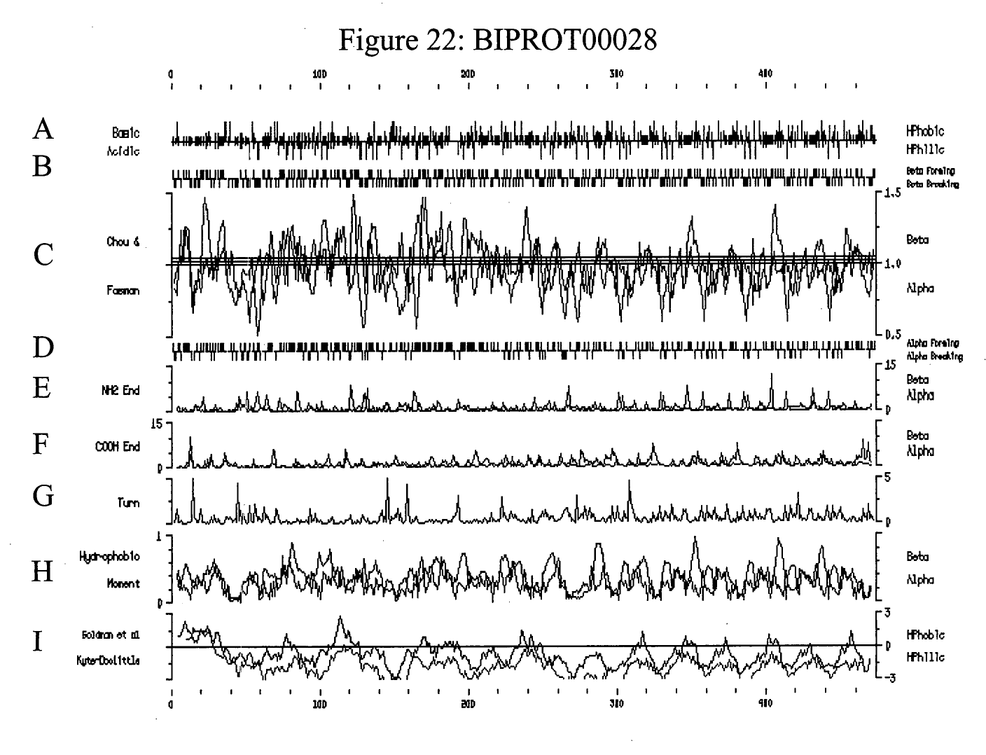

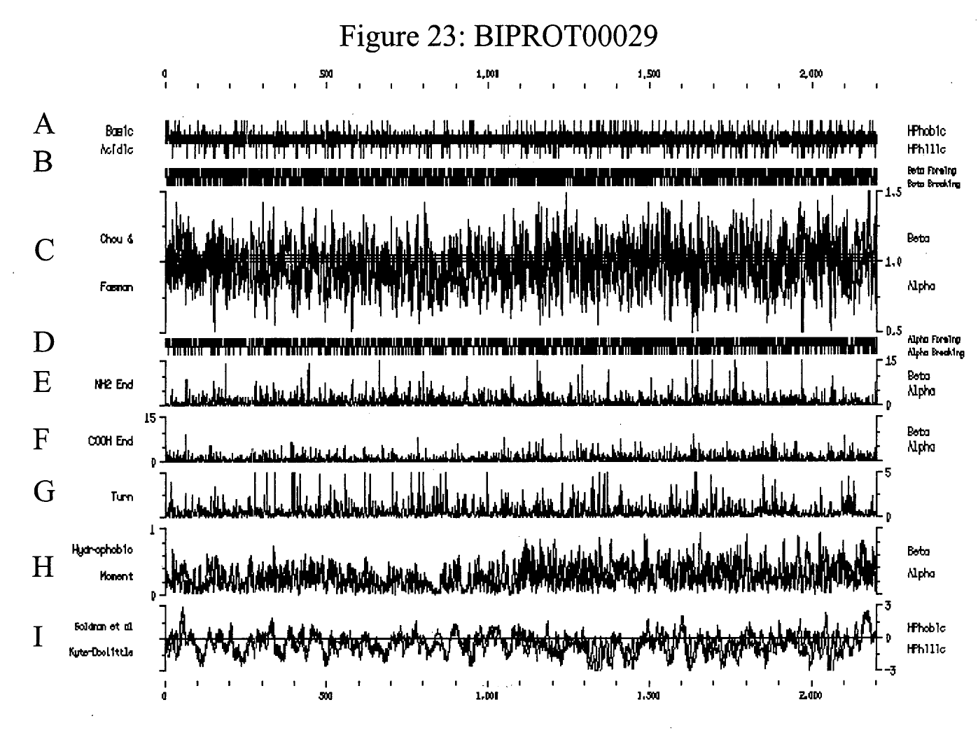

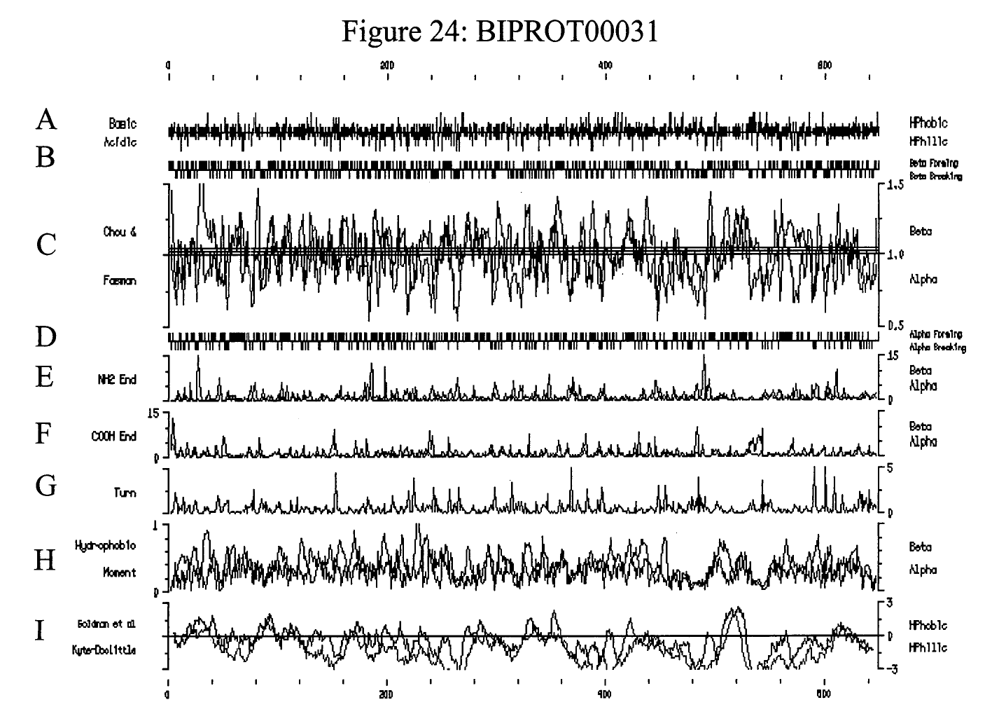

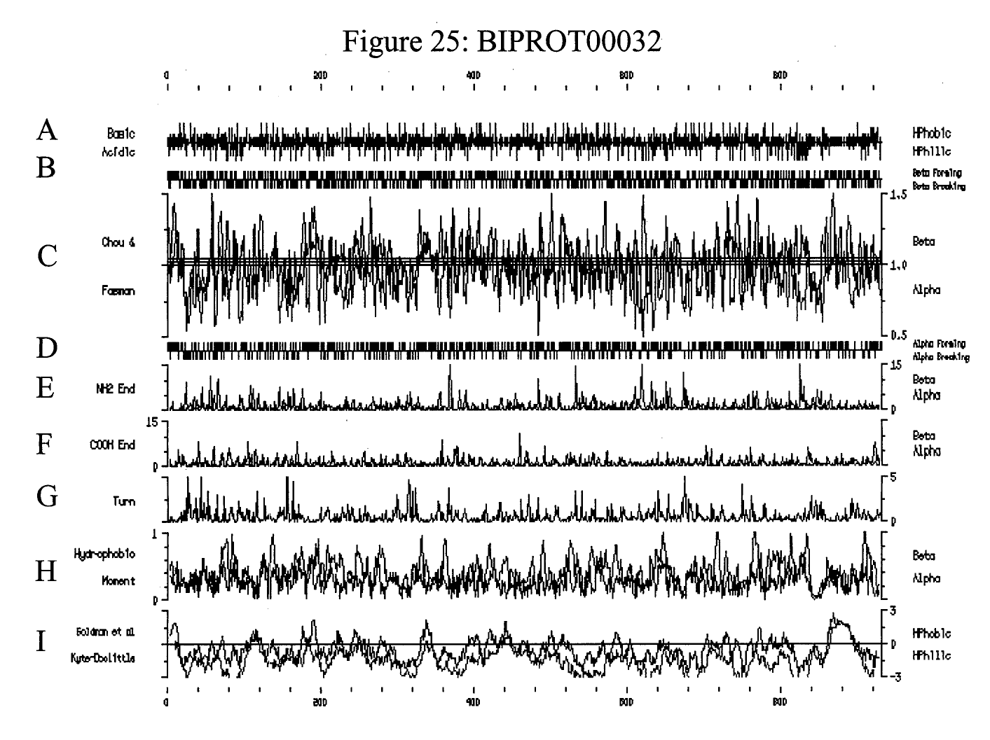

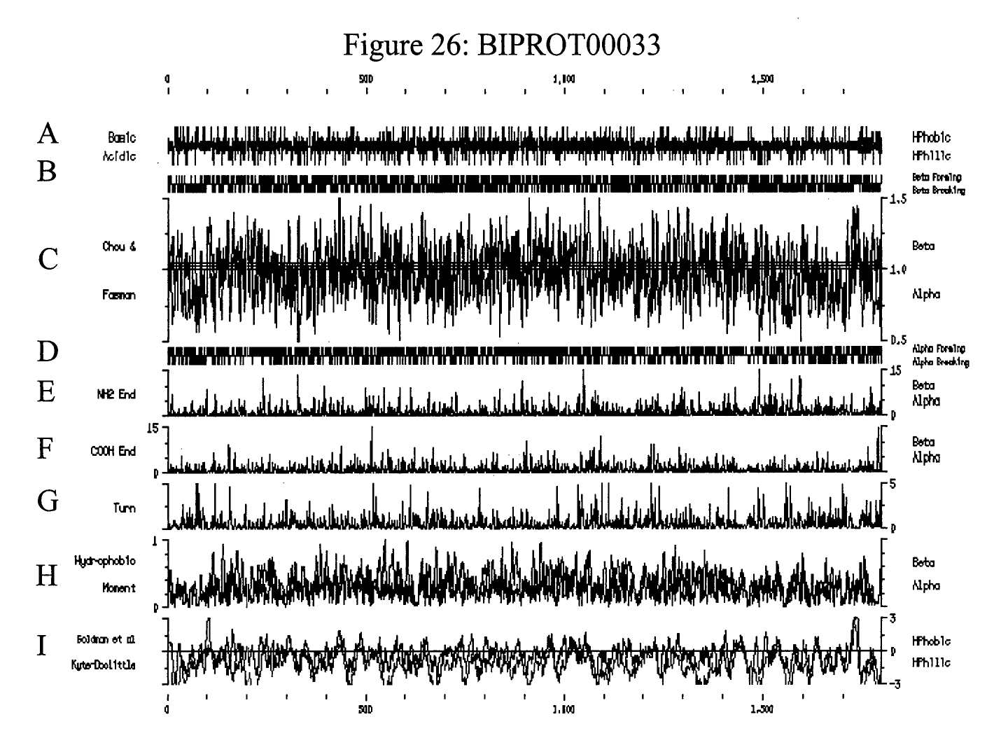

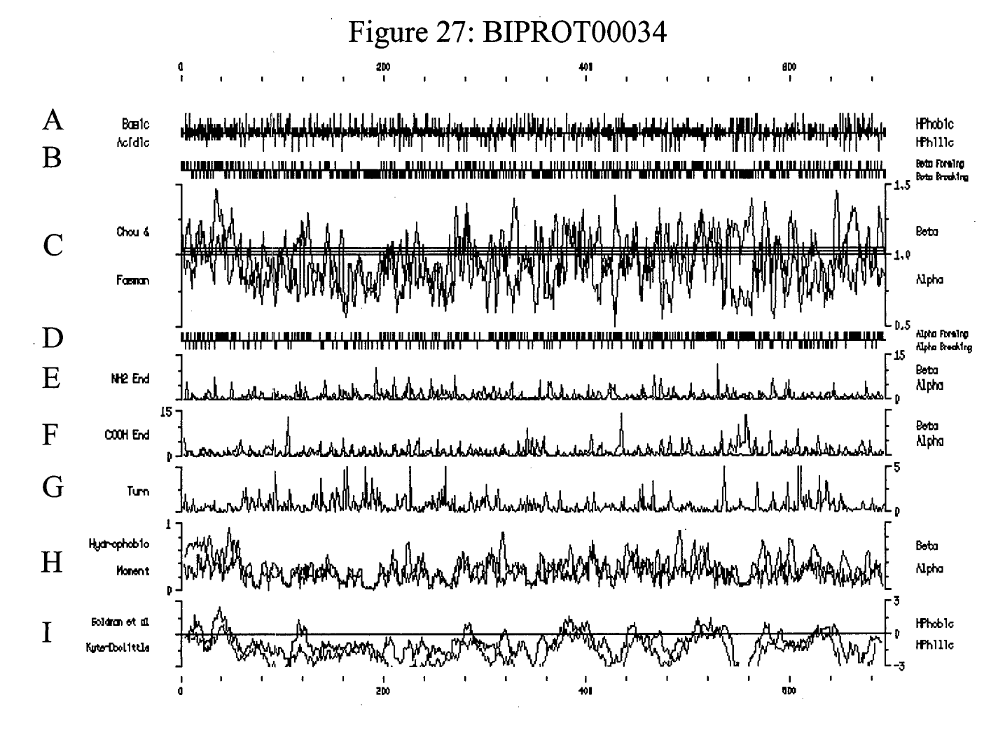

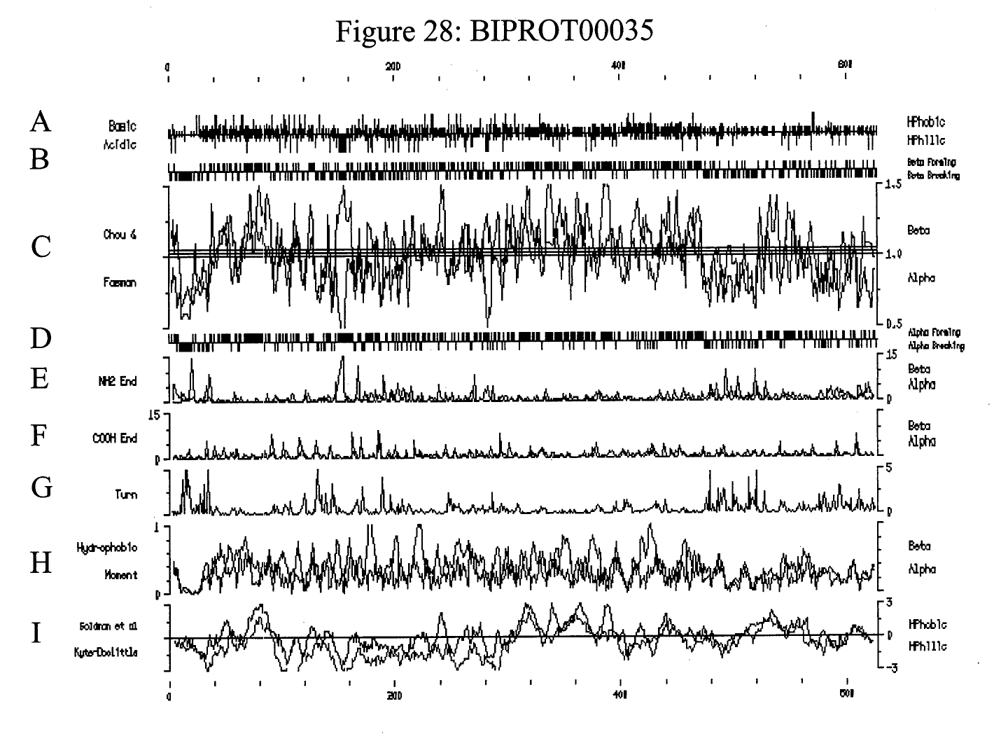

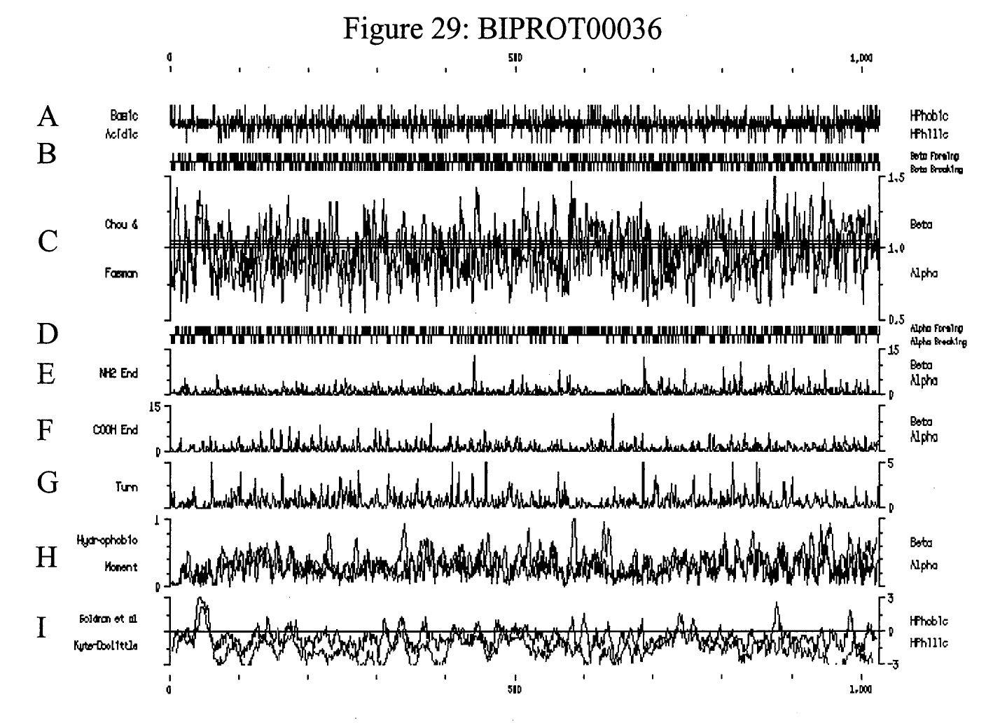

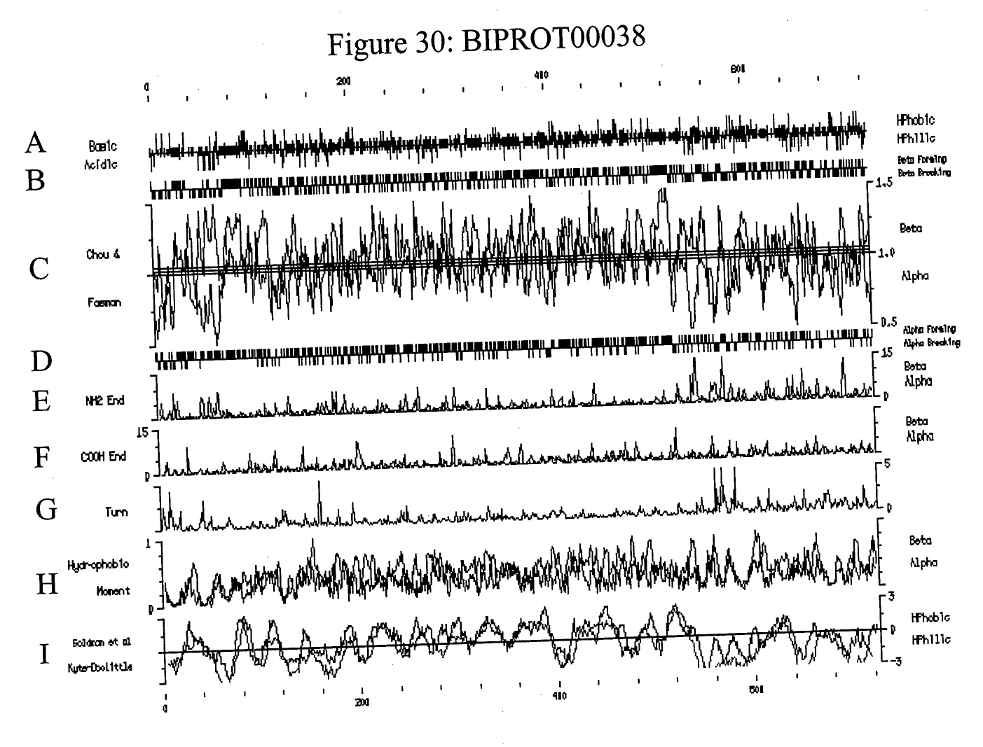

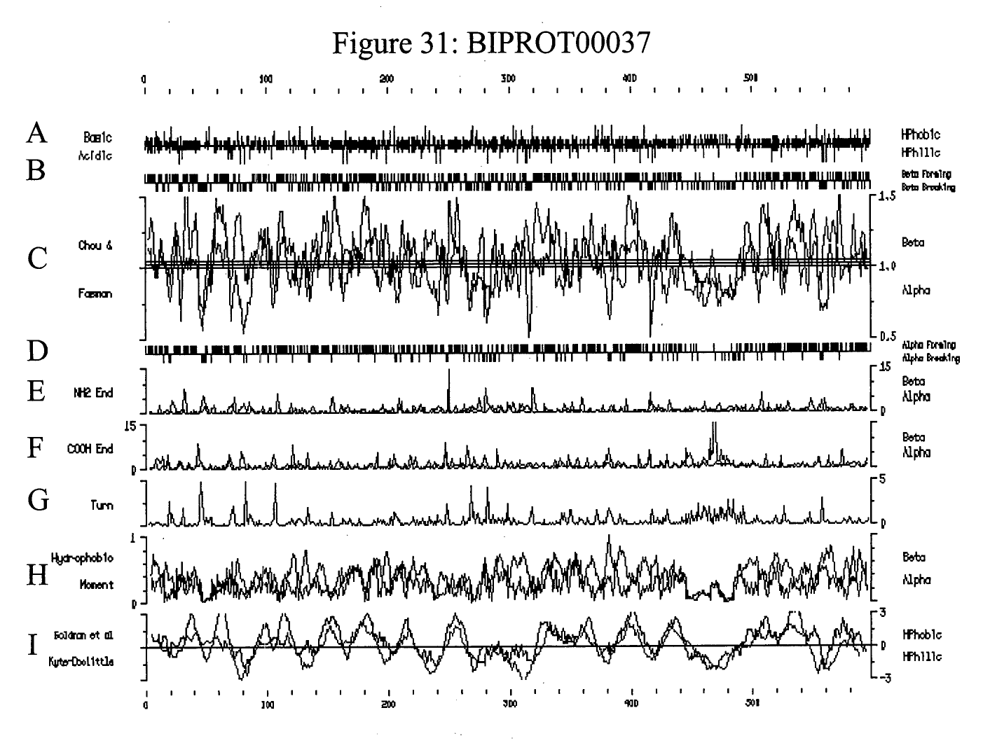

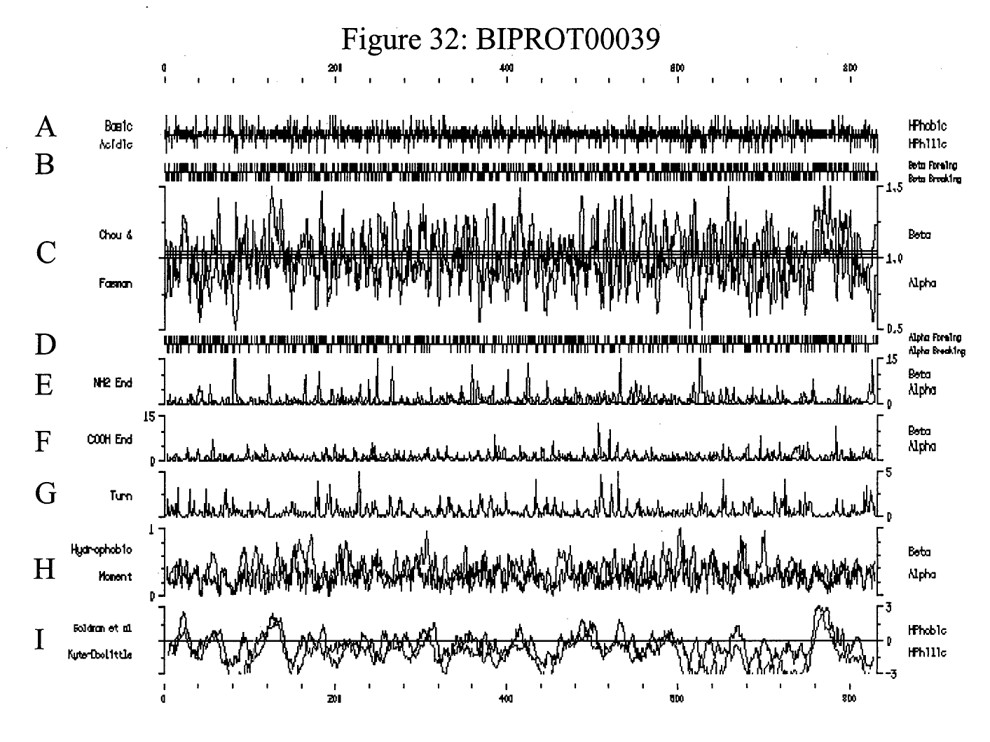

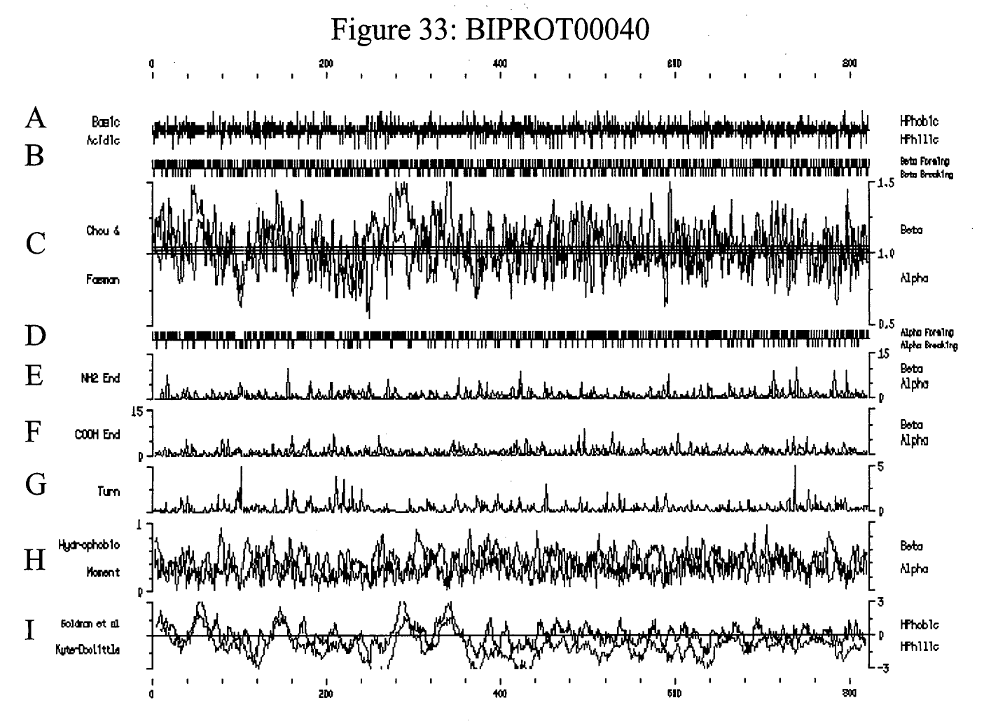

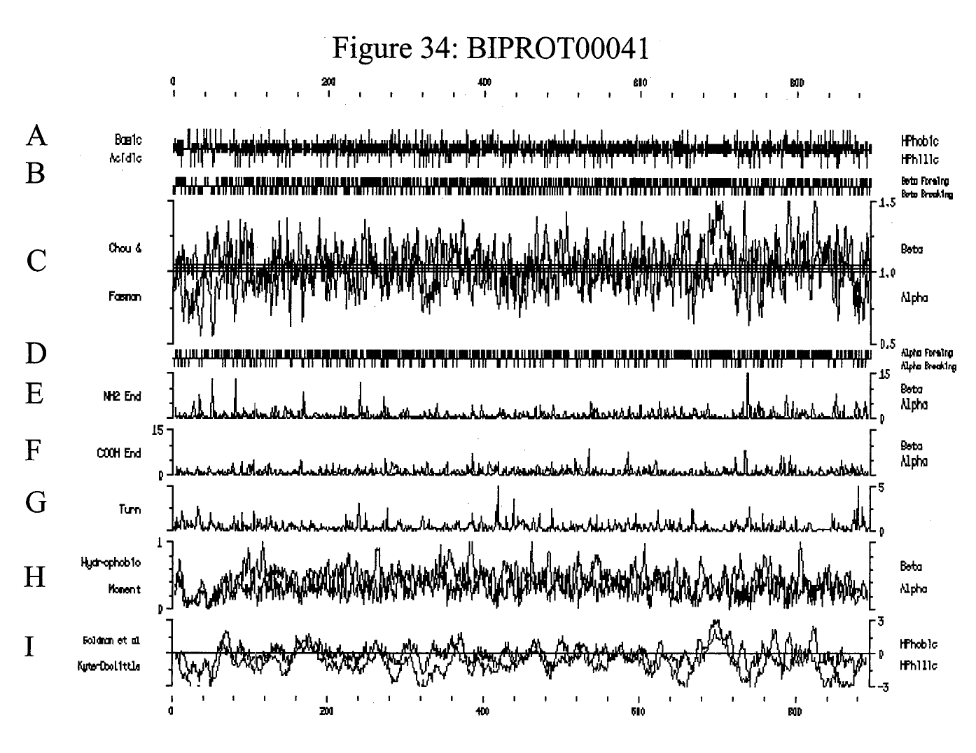

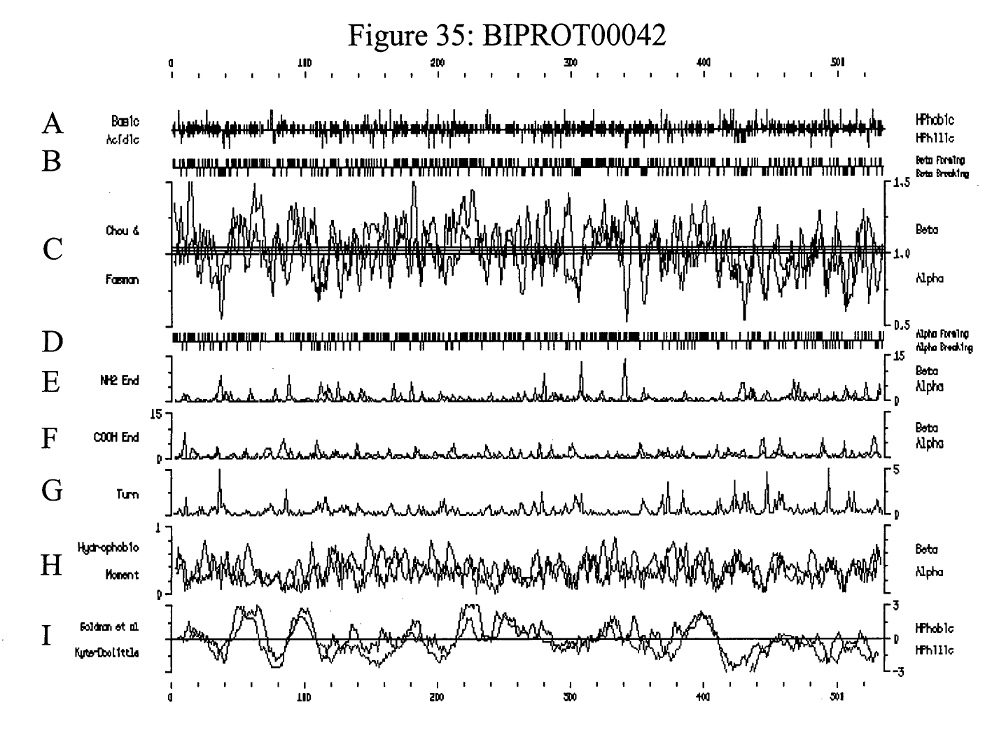

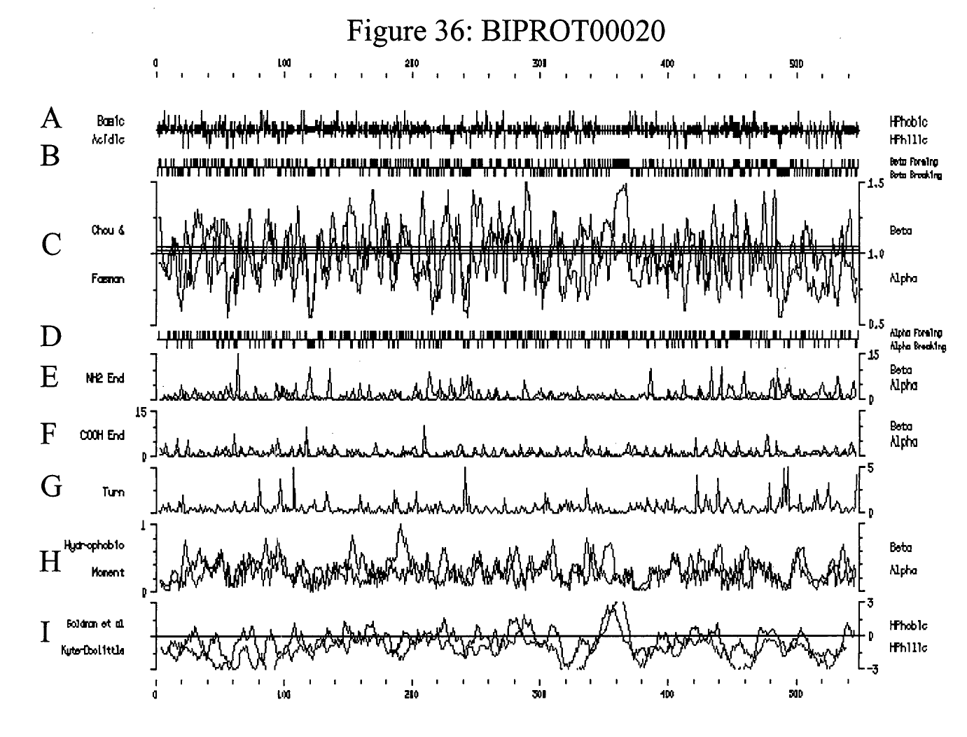

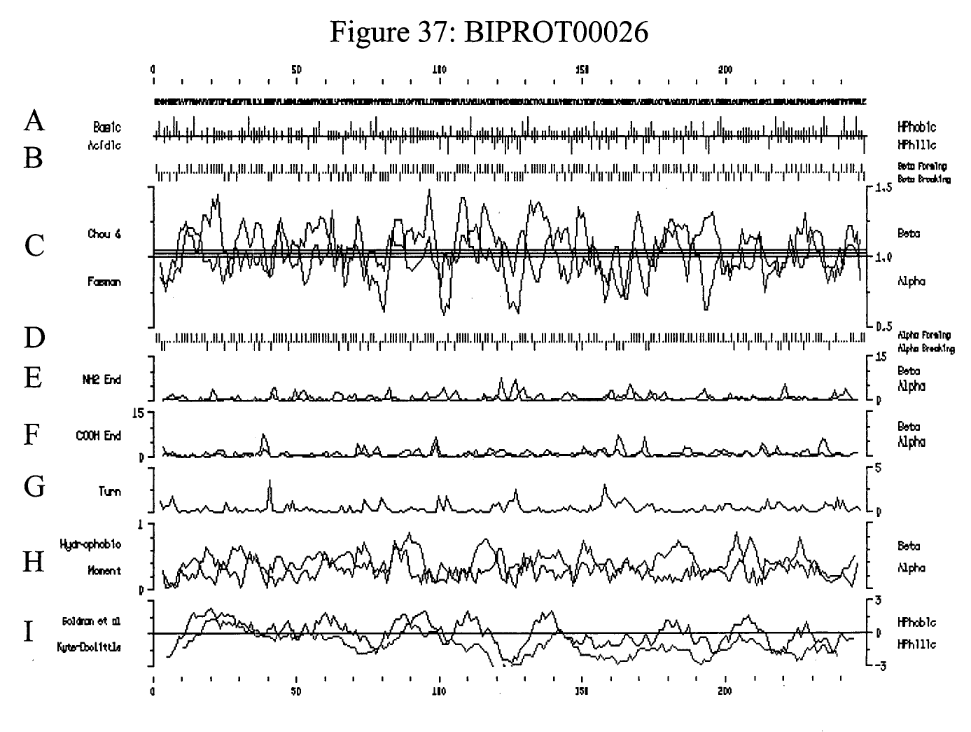

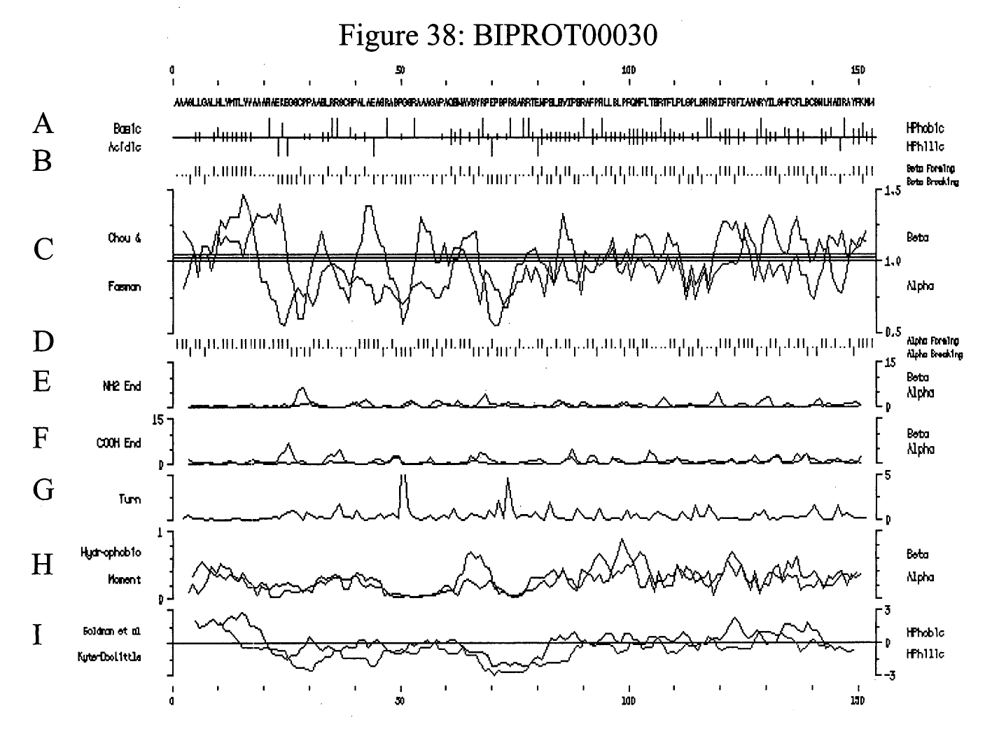

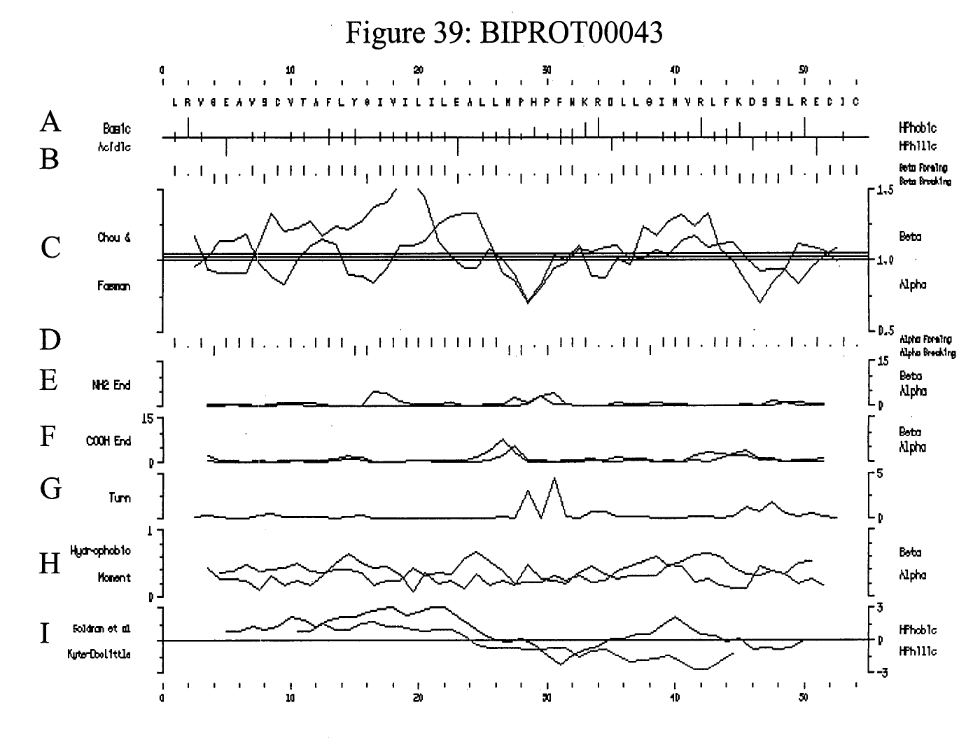

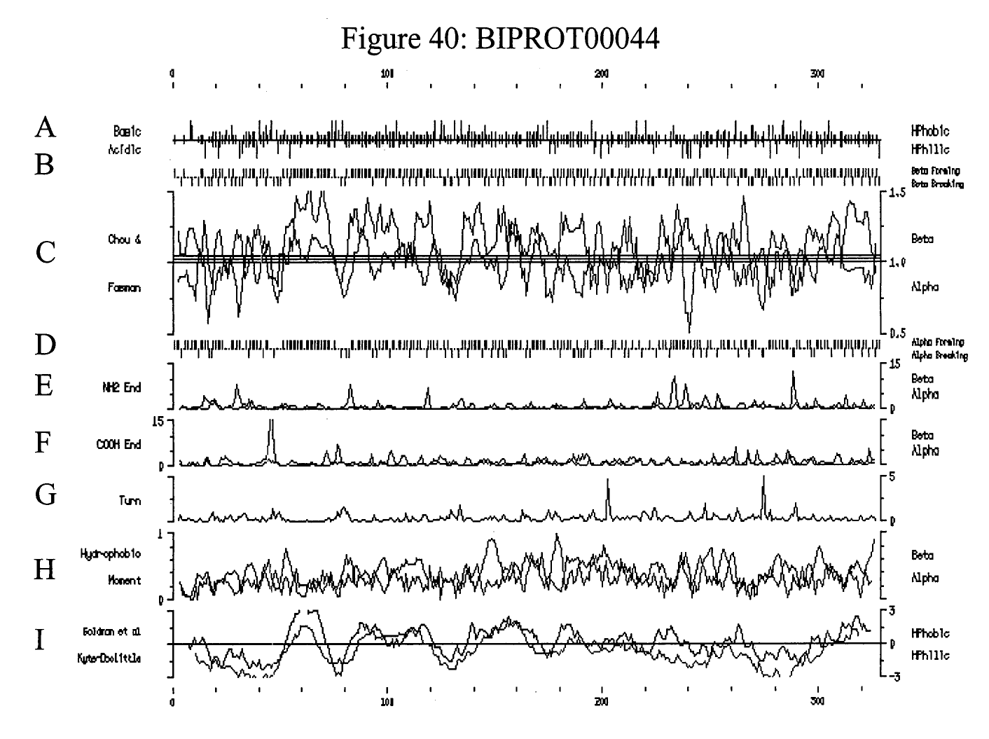

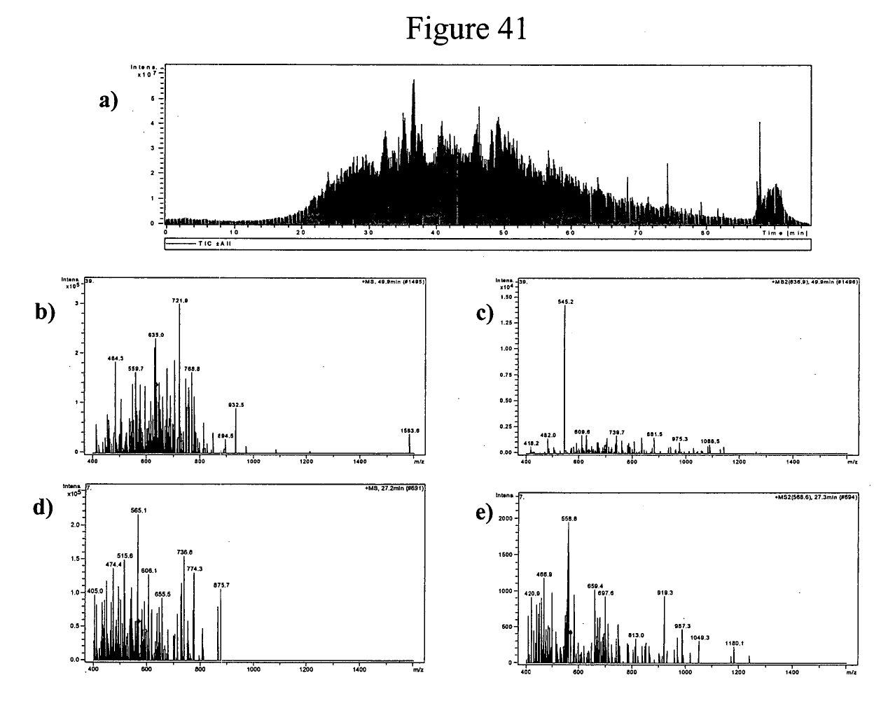

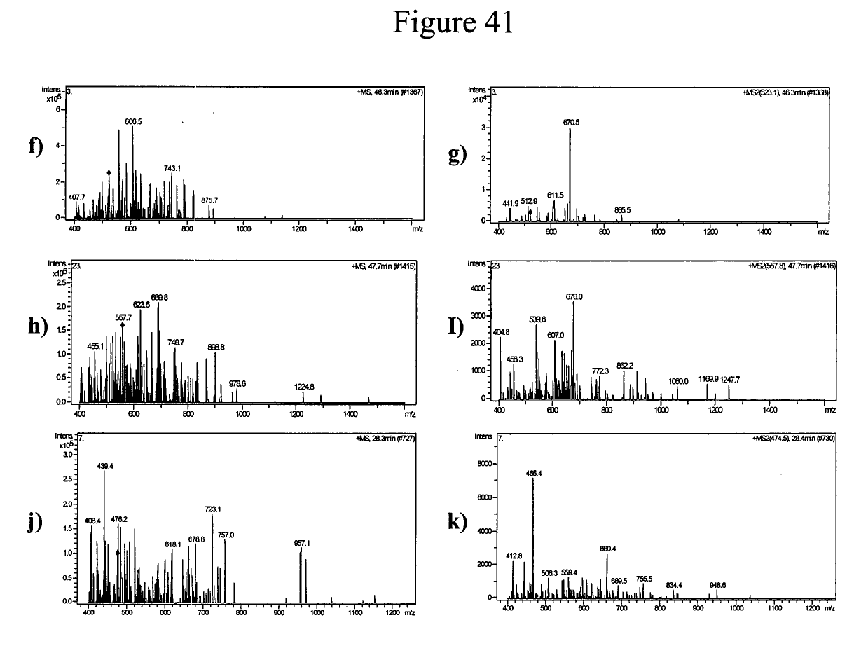

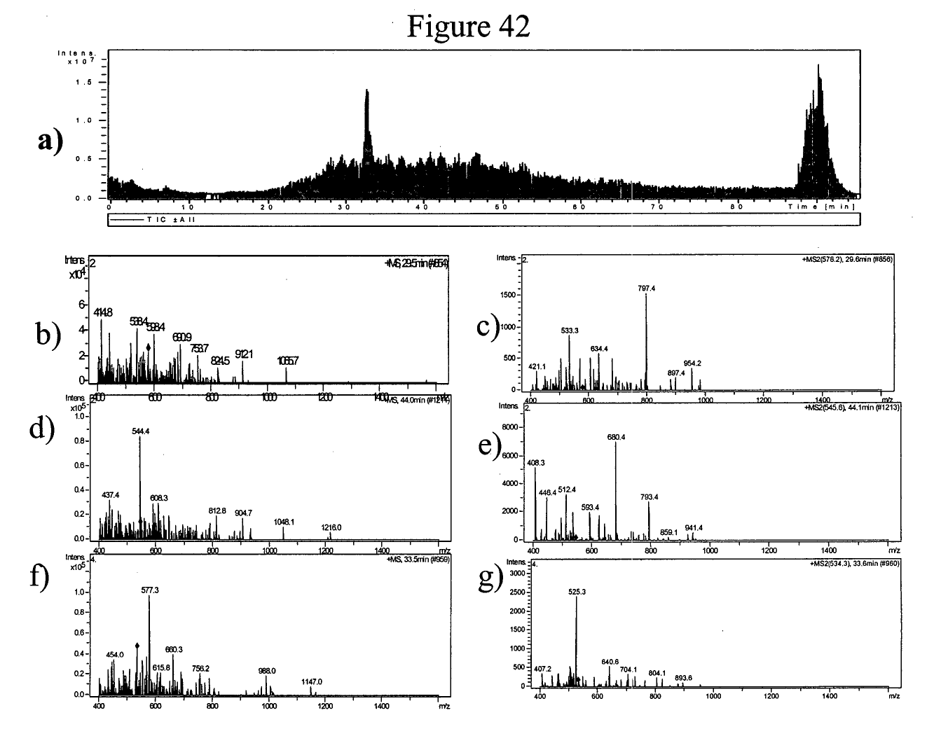

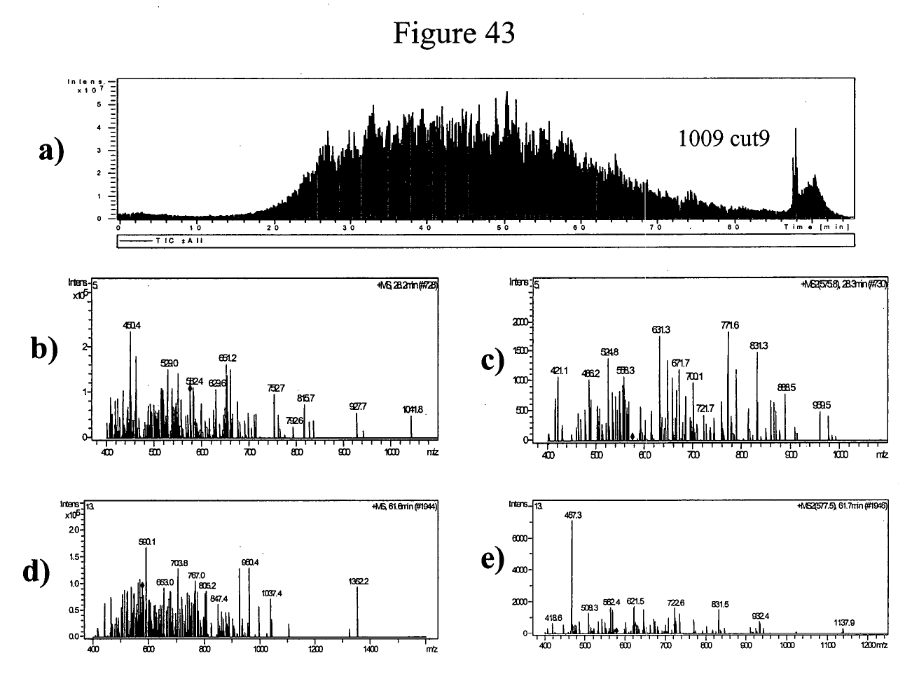

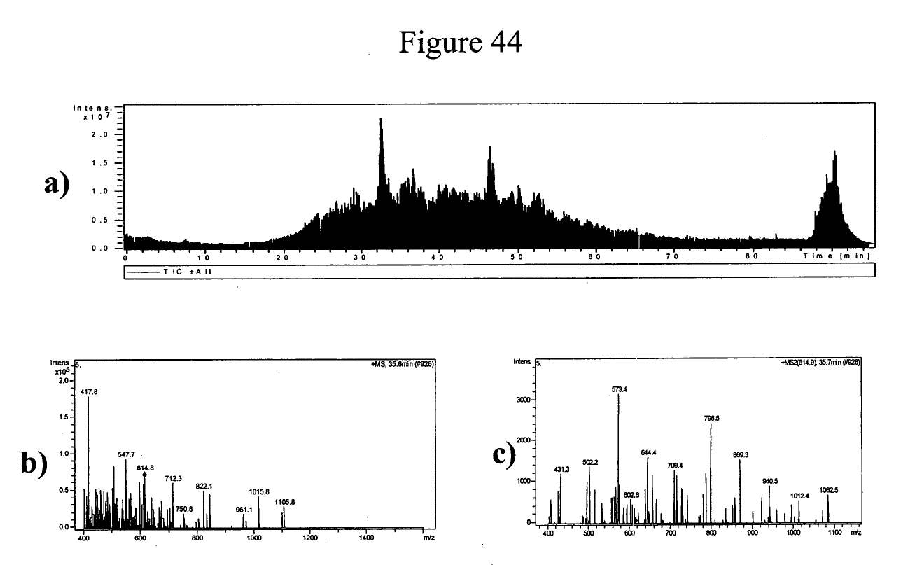

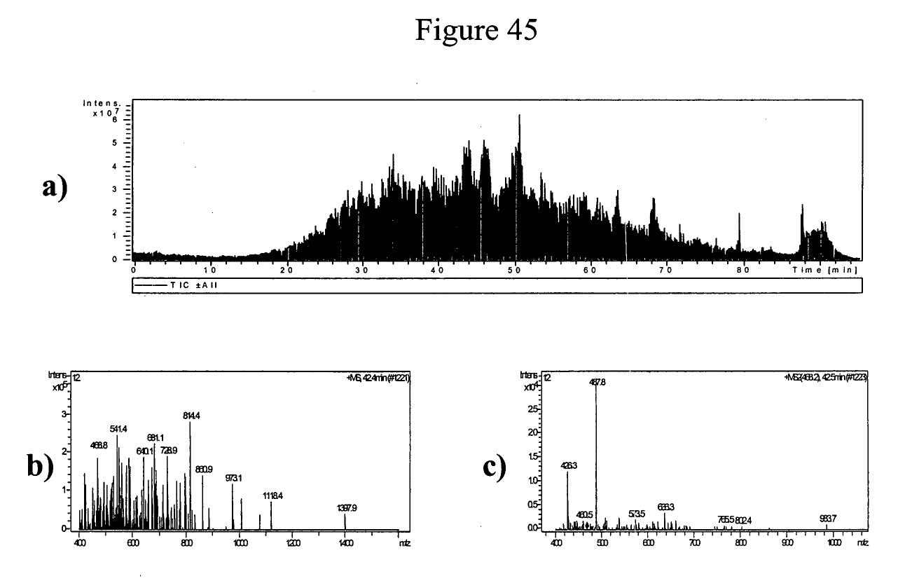

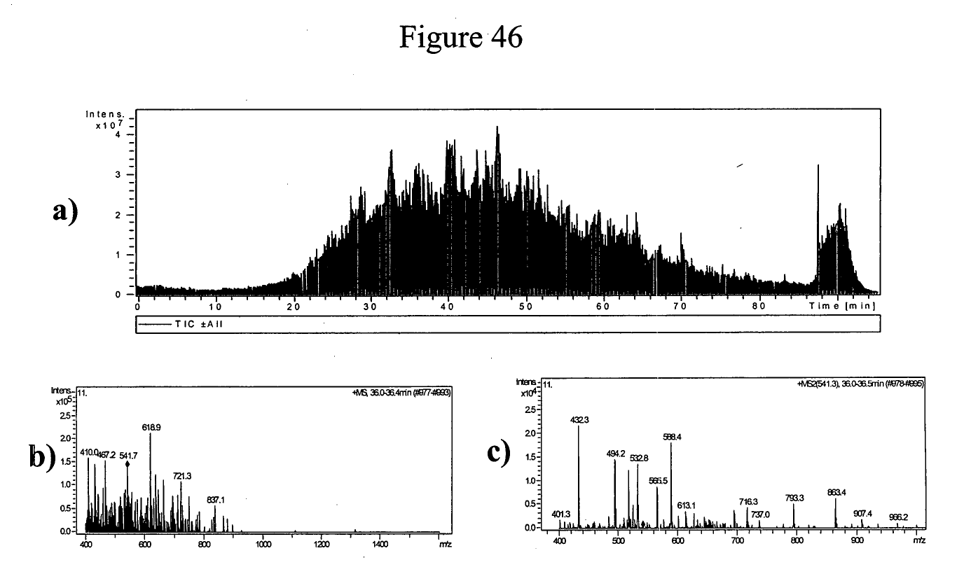

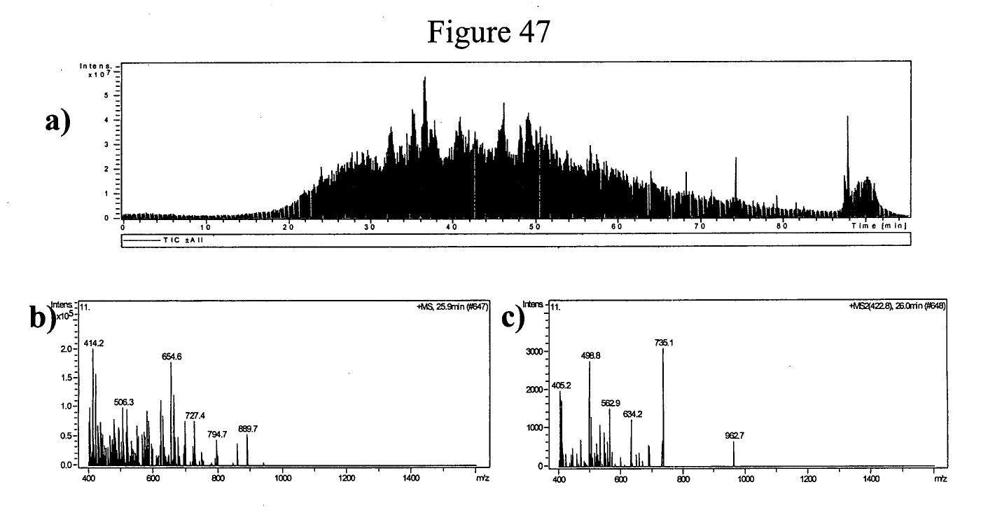

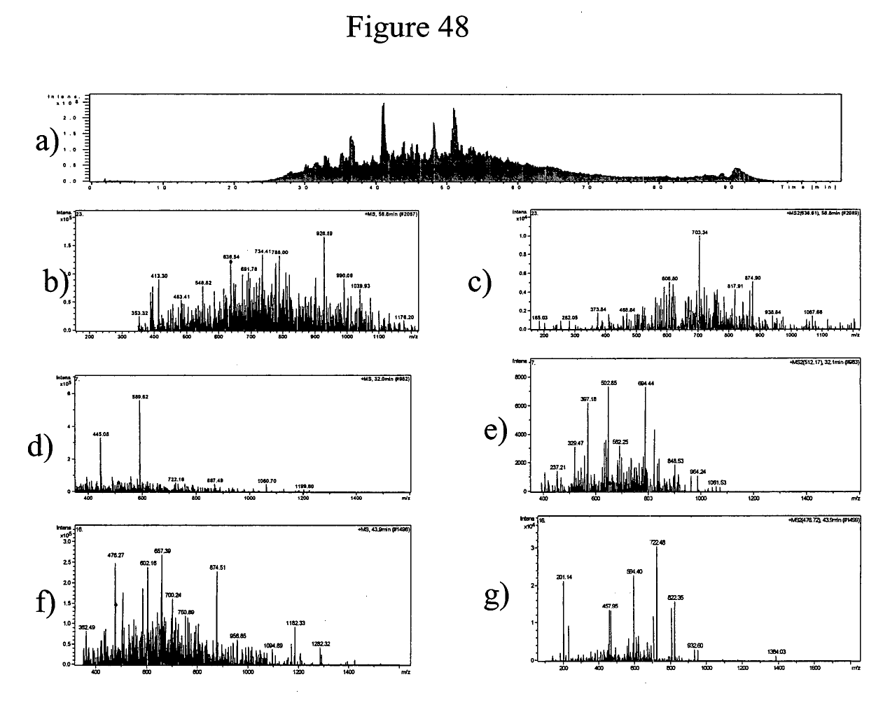

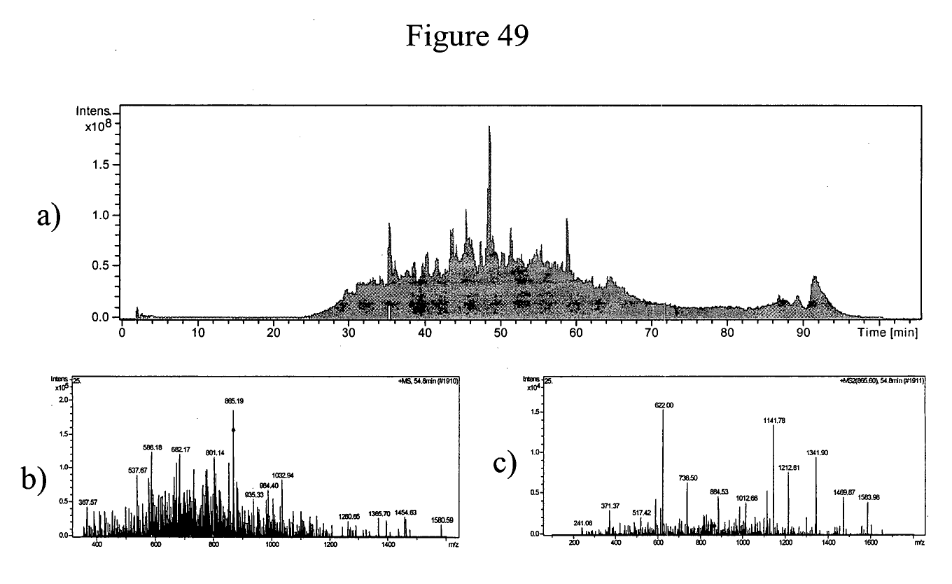

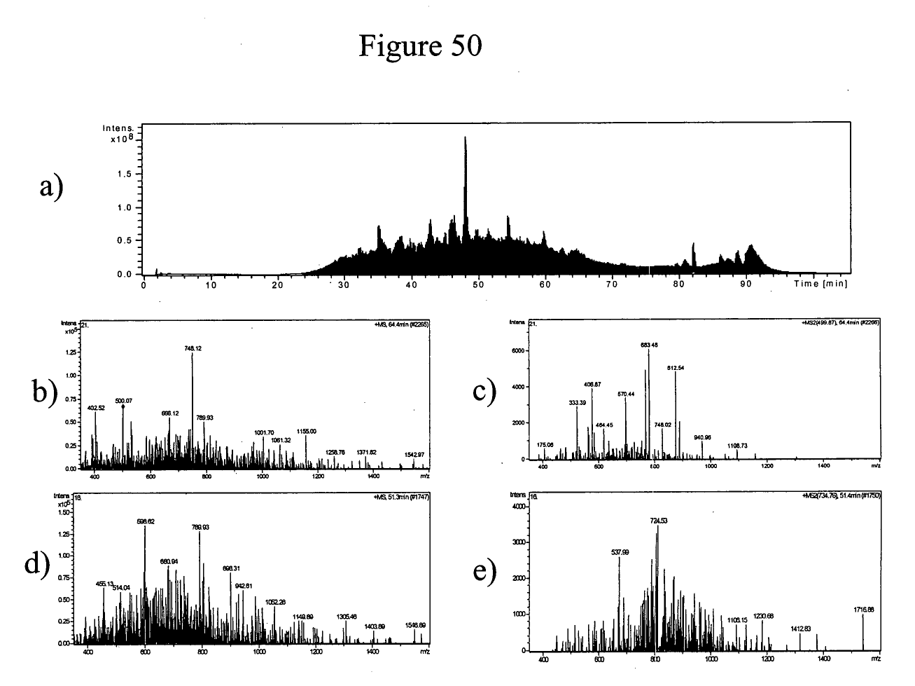

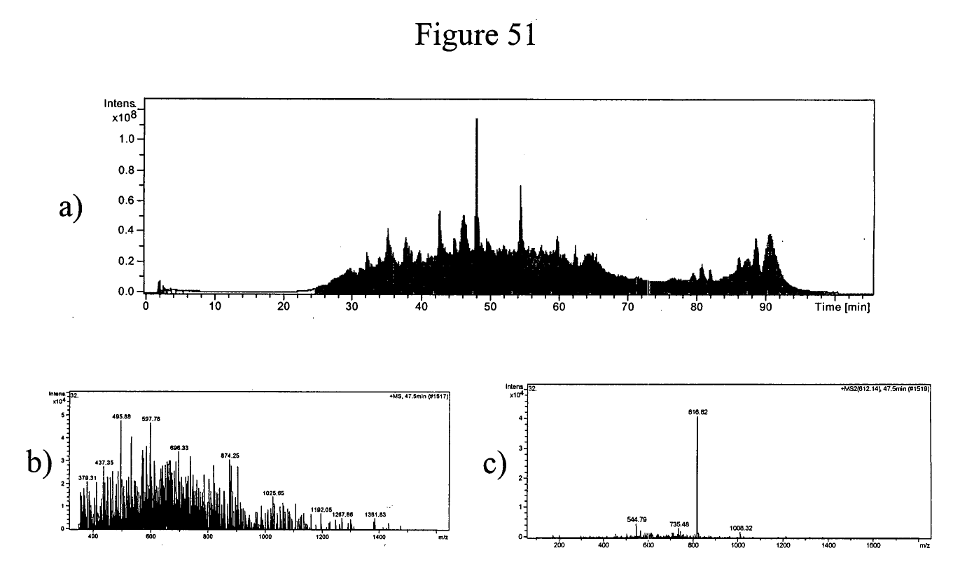

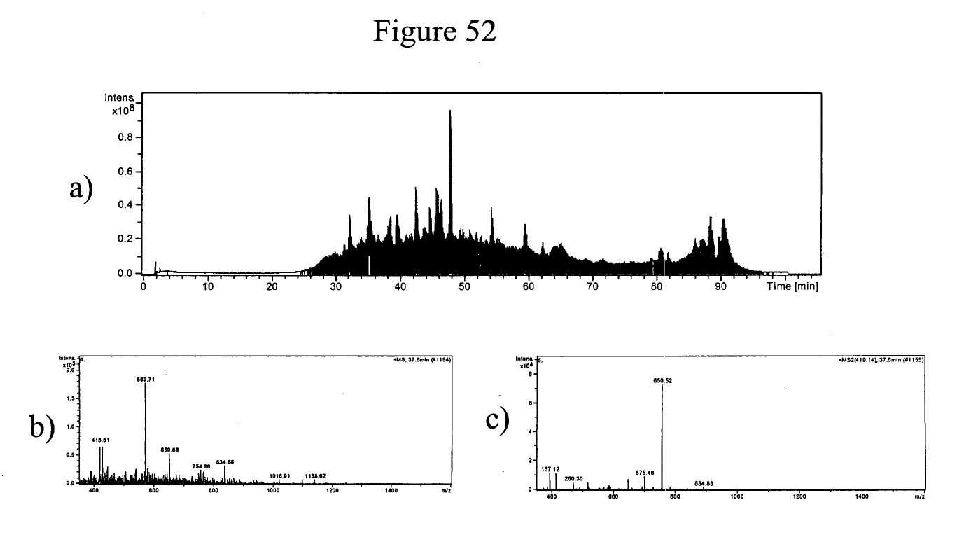

The separation column was a LC Packing Pepmap 3 um C18 0.3 mm=150 mm run at 5 μL/minute on an Agilent 1100 Capillary HPLC. The gradient went from 3% ACN 0.1% formic to 60% ACN 0.1% formic in 75 minutes. The reversed phase liquid chromatography column is coupled to the ion trap mass spectrometer. The Agilent MSD Ion Trap mass spectrometer was operated in positive ion mode, capillary voltage was 4 kV, nebulizer gas at 15 L/hr and the cone gas was at 5 L/hr. The MS/MS spectra were obtained in a data dependant manner with each mass-spectrometer scan yielding up too three MS/MS scans. Acquisitions parameters on the MS/MS acquisition were 30000 ions or 300 milliseconds. Exemplary data collected by the above methods are shown in FIGS. 1-12 and 41-52.

The MS/MS spectra were extracted from the raw data by using threshold values for the total ion counts. These extracted MS/MS spectra were then searched against the Acembly 33 database using the Mascot® (Matrix Science) alignment program. The following settings were used during the search: The enzyme was trypsin, the mass tolerances were 2 amu parent and 0.8 amu MS/MS scan, the peptide charge was two or three, and the variable modification was oxidation. The search results were parsed and the parsed data was then imported into an Excel spreadsheet for comparison and later quantitation. The proteins identified by this method are shown in Tables 1 and 2. Table 7 indicates the actual peptide sequence that was obtained from the MS/MS data and the corresponding SEQ ID NO for certain lung tumor-associated polypeptides.

TABLE 7

| | |
|---|---|
| Corresponding | SEQ |

| | | | | |
|---|---|---|---|---|
| full-length | Gel | MS | MS/MS | ID |

| | | | | | | |
|---|---|---|---|---|---|---|
| SEQ ID NO: | slice | Tumor | (min) | (m/z) | NO: | Identified Peptide |

| | | | | | | |
|---|---|---|---|---|---|---|
| 25 | 14 | 2 | 64.4 | 499.87 | 53 | GEELRTHR |
| 26 | 10 | 3 | 25.7 | 410.5 | 54 | ESSGGEVR |
| 27 | 9 | 1 | 28.3 | 575.8 | 55 | WLDACLAGSR |
| 28 | 17 | 1 | 29.6 | 578.2 | 56 | SDGVYTGLSTR |
| 29 | 5 | 1 | 36.6 | 541.9 | 57 | VVSAGRGEAVTCQGAR |
| 30 | 7 | 1 | 27.3 | 568.6 | 58 | SKSSSTTYKF |
| 31 | 7 | 1 | 46.3 | 523.1 | 59 | KPQIDSNKSNNYR |
| 32 | 10 | 3 | 33.4 | 474.1 | 60 | DQEELKGK |
| 33 | 14 | 1 | 35.6 | 614 | 61 | LMAEGAPKWPK |
| 34 | 16 | 2 | 37.6 | 419.4 | 62 | FLAENNK |
| 35 | 14 | 2 | 51.3 | 734.76 | 63 | AHSQLSVLPAAGCR |
| 36 | 7 | 1 | 49.4 | 636.9 | 64 | FPGEEGTTNSFLKARPR |
| 37 | 11 | 3 | 50.5 | 555.7 | 65 | MTNNGGYKAR |
| 38 | 7 | 1 | 47.7 | 557.8 | 66 | EGGCPPAASLR |
| 39 | 10 | 2 | 56.8 | 636.61 | 67 | IWPTTKRPAITPANNPK |
| 40 | 15 | 2 | 47.5 | 612.1 | 68 | EGQYARLISPPVHLPR |
| 41 | 10 | 2 | 32.1 | 512.17 | 69 | FGLRAIVADPVTFK |
| 42 | 7 | 1 | 25.9 | 422.8 | 70 | WVFVVRVLSVHAVEK |
| 43 | 10 | 3 | 44.5 | 519.8 | 71 | LSIPVMVVTLDPTR |
| 44 | 11 | 1 | 42.4 | 468.2 | 72 | CSCKPGYQGEGR |
| 45 | 7 | 1 | 28.3 | 474.5 | 73 | VLYVISSLLSSLK |
| 46 | 9 | 1 | 61.6 | 577.5 | 74 | YGTPATSGRDK |
| 47 | 17 | 1 | 44.1 | 545.6 | 75 | SEDYGKNFK |
| 48 | 11 | 2 | 54.8 | 565.6 | 76 | QINLNNEWTVEKLK |
| 49 | 17 | 1 | 33.6 | 534.3 | 77 | EDTSASETAR |
| 50 | 10 | 2 | 43.9 | 476.7 | 78 | EVTMELTK |
| 51 | 15 | 3 | 25.2 | 459.2 | 79 | DLLGIMVR |
| 52 | 9 | 3 | 36.7 | 594.4 | 80 | RMISNRWER |

### Example 3—Quantitation Method

For each peptide identified in the tumor samples the m/z and retention times were used to look up the intensity values from the MS/MS data as follows. A window was measured around the observed m/z and retention times to include the peptide but exclude noise as much as possible. Within this window intensities were gathered for each retention time. The mass dimension was collapsed. For each retention time that had multiple intensities, those intensities were averaged. This produces data in two dimensions, retention time and intensity, which is then quantified as a curve using trapezoidal approximation. This quantity was used as the tumor peptide quantity. Those same mass and retention times were then used to quantitate the appropriate region in the corresponding cut of the matched normal sample. The ratio of these quantities was used as the fold change determination. Proteins that were at least 2.9 fold upregulated in 2 or more tumors as compared to matched normals were input into the TMHMM transmembrane prediction server (see e.g. URL: cbs.dtu.dk/services/TMHMM on the world wide web) for transmembrane predictions. Proteins containing transmembrane domains were selected for validation of the mass spectroscopy data. This data is shown in Table 8.

TABLE 8

| | Tumor | Normal | |
|---|---|---|---|
| SEQ ID NO: | Quantity | Quantity | Ratio |
| 25 | 103879.21 | 18266.04 | 5.69 |
| 26 | 49391.39 | 15077.47 | 3.28 |
| 27 | 114940.78 | 33482.71 | 3.43 |
| 28 | 35885.32 | 11284.28 | 3.71 |
| 29 | 510596.50 | 32062.05 | 15.93 |
| 30 | 82528.12 | 14203.83 | 5.81 |
| 31 | 272479.62 | 62291.23 | 4.37 |
| 32 | 58650.07 | 16981.47 | 3.45 |
| 33 | 112025.56 | 34588.58 | 3.24 |
| 34 | 288288.01 | 26092.34 | 11.05 |
| 35 | 57843.80 | 12911.69 | 4.14 |
| 36 | 131107.15 | 4146.03 | 31.62 |
| 37 | 88080.04 | 25692.22 | 3.43 |
| 38 | 178470.12 | 43179.50 | 4.13 |
| 39 | 131510.00 | 33353.12 | 3.94 |
| 40 | 79561.84 | 22159.53 | 3.59 |
| 41 | 1022543.28 | 106157.67 | 12.68 |
| 42 | 84278.38 | 28496.22 | 4.21 |
| 43 | 35976.42 | 5546.18 | 6.49 |
| 44 | 360144.82 | 36824.34 | 9.78 |
| 45 | 88317.32 | 26418.49 | 3.34 |
| 46 | 281668.00 | 51751.35 | 5.44 |
| 47 | 120130.30 | 18510.18 | 6.58 |
| 48 | 263299.43 | 41809.94 | 6.30 |
| 49 | 38232.82 | 10700.17 | 3.57 |
| 50 | 439061.93 | 60506.06 | 7.26 |
| 51 | 44961.01 | 10466.76 | 4.30 |
| 52 | 208132.61 | 21538.26 | 9.66 |

### Example 4—Validation

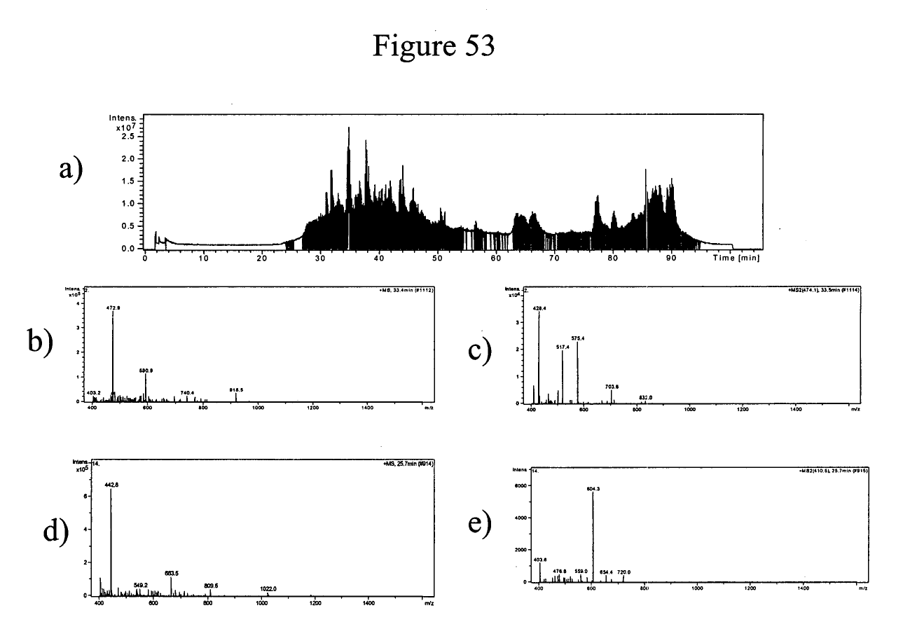

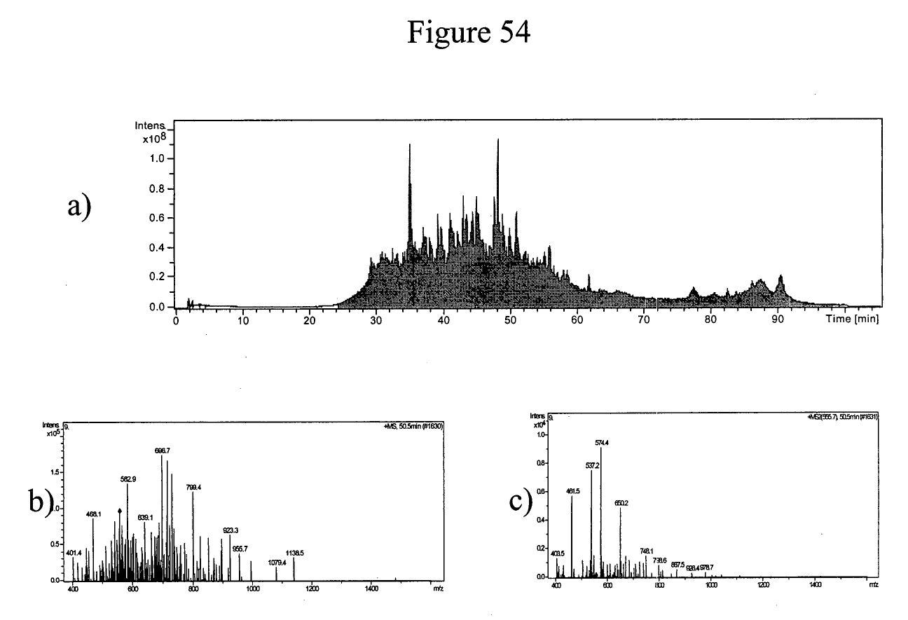

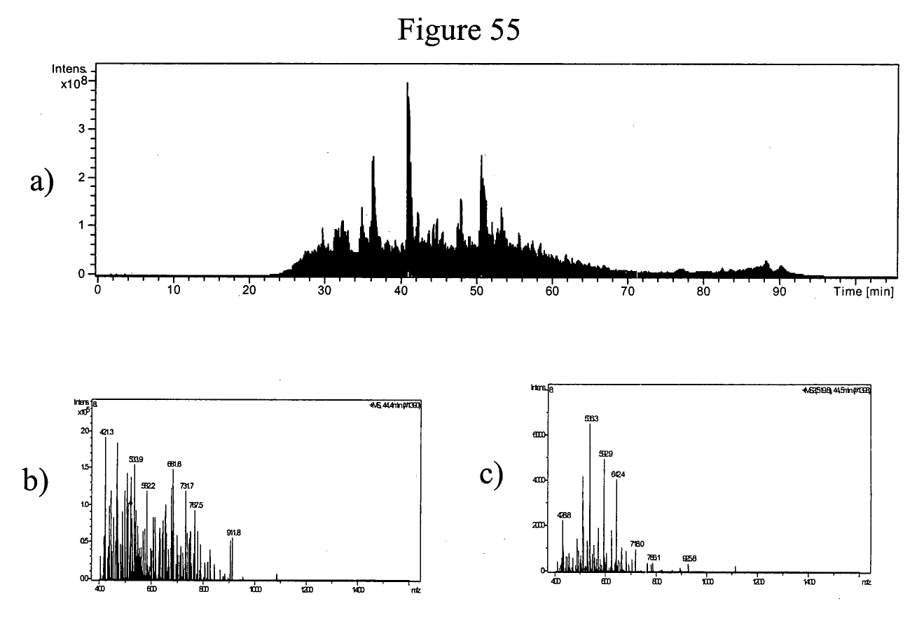

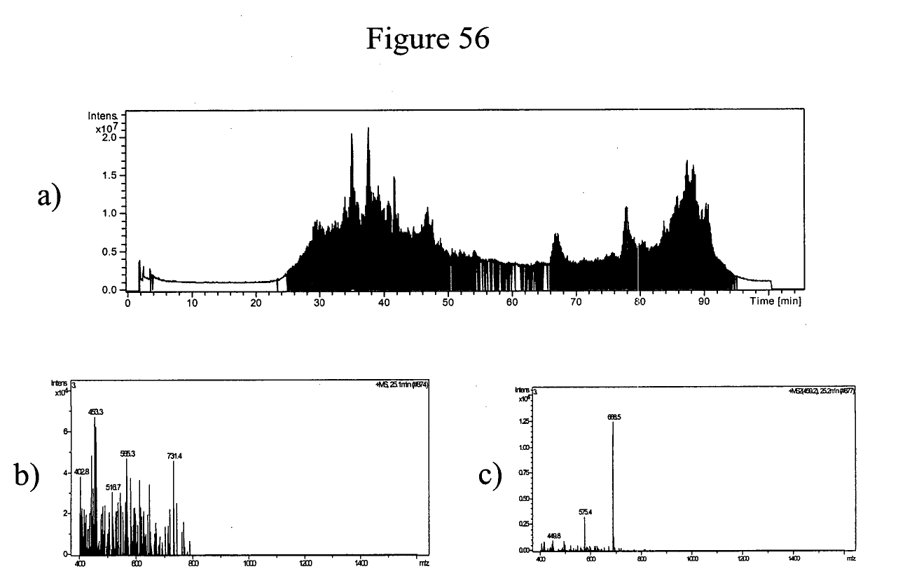

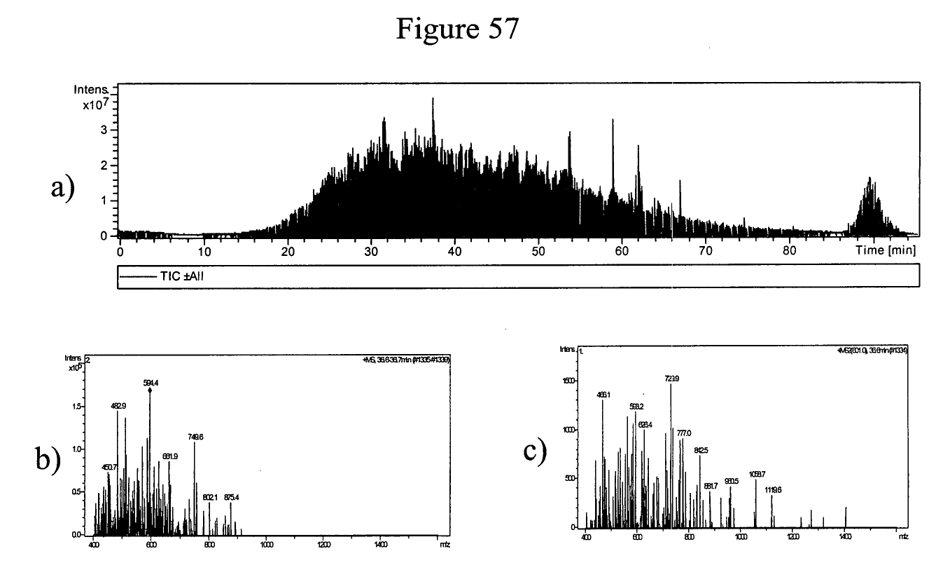

The validation process includes manual verification of the expression levels and mascot alignments. The expression ratios for each peptide identified or cross quantitated was compared manually to the corresponding matched sample and then compared to the expression ratio calculated by the quantitation software. Where a protein was identified in multiple cuts, the sum of the areas for all the cuts the peptide was determined and compared. The MS/MS spectra were searched by mascot again and the alignment manually verified by inspection. Data for peptides that were validated are shown in FIGS. 1-12 and 41-57.

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

1. A method for treating a hyperproliferative disorder in an animal, comprising administering to an animal in need of treatment a composition comprising a binding molecule which specifically binds to a variant polypeptide, or fragment thereof, which is at least 90% identical to a lung tumor-associated polypeptide selected from the group consisting of: SEQ ID NO: 1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; and SEQ ID NO:52.

2-5. (canceled)

6. The method of claim 1, wherein said variant polypeptide, or fragment thereof, is at least 95% identical to said lung tumor-associated polypeptide.

7. The method of claim 6, wherein said variant polypeptide, or fragment thereof, is at least 100% identical to said lung tumor-associated polypeptide.

8. (canceled)

9. The method of claim 1, wherein said variant polypeptide is fused to a heterologous polypeptide.

10. The method of claim 9, wherein said heterologous polypeptide is an immunoglobulin Fc domain.

11. The method of claim 1 wherein said binding molecule is an antibody or immunospecific fragment thereof.

12-18. (canceled)

19. The method of claim 11, wherein said antibody or fragment thereof is monoclonal.

20. The method of claim 11, wherein said antibody or fragment thereof is multispecific, comprising at least two non-identical antigen binding domains.

21-24. (canceled)

25. The method of claim 19, wherein said antibody or fragment thereof is humanized.

26-34. (canceled)

35. The method of claim 1, wherein said binding molecule is conjugated to an agent selected from the group consisting of: a cytotoxic agent, a therapeutic agent, a cytostatic agent, a biological toxin, a prodrug, a peptide, a protein, an enzyme, a virus, a lipid, a biological response modifier, a pharmaceutical agent, a lymphokine, a heterologous antibody or fragment thereof, a detectable label, and polyethylene glycol (PEG).

36-37. (canceled)

38. The method of claim 1, wherein said hyperproliferative disorder is selected from the group consisting of a neoplasm, a tumor, a malignancy, or a metastasis thereof.

39-49. (canceled)

50. The method of claim 1, wherein said hyperproliferative disease is cancer, said cancer selected from the group consisting of: epithelial squamous cell cancer, melanoma, leukemia, myeloma, lung cancer, pancreatic cancer, cervical cancer, ovarian cancer, liver cancer, bladder cancer, breast cancer, colon cancer, renal cancer, prostate cancer, testicular cancer, thyroid cancer, and head and neck cancer.

51-53. (canceled)

54. The method of claim 11, wherein said antibody or fragment thereof is produced by the method comprising:
(a) immunizing a mammal with an immunogen comprising a lung tumor-associated polypeptide, or fragment thereof, selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO: 10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; and SEQ ID NO:52;
(b) fusing spleen cells of said mammal with immortalized cells to produce a hybridoma library;
(c) screening said hybridoma library for an antibody which specifically binds to said lung tumor-associated polypeptide, or fragment thereof, of step (a); and
(d) recovering the hybridoma which produces said antibody.

55. The method of claim 54, wherein said antibody production method further comprises:
(e) isolating cDNA molecules which encode the heavy chain and light chain of said antibody from the hybridoma of (d);
(f) introducing said cDNA molecules into a host cell capable of expressing said antibody; and
(g) expressing said antibody from said host cell.

56. The method of claim 55, wherein said antibody production further comprises
(h) engineering said cDNA molecules of (e) such that they express a lung tumor-associated polypeptide specific antibody or fragment thereof with a characteristic selected from the group consisting of: reduced immunogenicity in a human, increased binding affinity, or a combination thereof.

57. (canceled)

58. A method of detecting abnormal hyperproliferative cell growth in a patient comprising:
(a) obtaining a biological sample from the patient;
(b) contacting said sample with a binding molecule which specifically binds to a variant polypeptide, or fragment thereof, which is at least 90% identical to a lung tumor-associated polypeptide selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; and SEQ ID NO:52 and
(c) assaying the expression level of said lung tumor-associated polypeptide in said sample.

59-63. (canceled)

64. The method of claim 58, wherein said variant polypeptide, or fragment thereof, is at least 100% identical to said lung tumor-associated polypeptide.

65-68. (canceled)

69. A method of diagnosing a hyperproliferative disease or disorder in a patient, comprising:
(a) administering to said patient a sufficient amount of a detectably labeled binding molecule which specifically binds to a variant polypeptide, or fragment thereof, which is at least 90% identical to a lung tumor-associated polypeptide selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; and SEQ ID NO:52;
(b) waiting for a time interval following said administration to allow said binding molecule to contact said variant polypeptide, or fragment thereof; and
(c) detecting the amount of said binding molecule bound to said variant polypeptide of fragment thereof in said patient.

70-74. (canceled)

75. The method of claim 69, wherein said binding molecule specifically binds to a polypeptide, or a fragment thereof, which is at least 100% identical to a lung tumor-associated polypeptide, or fragment thereof.

76-82. (canceled)

83. The method of claim 69, wherein said binding molecule is an antibody or immunospecific fragment thereof.

84-123. (canceled)
